SEQUENCE LISTING (26 sequences)

SEQ ID NO: 1 - DNA, 2727 bp, Cytomegalovirus sp.
  source 1..2727: mol_type = genomic DNA
atggaaagccggatctggtgcctggtcgtgtgcgtgaacctgtgcatcgtgtgcctgggagccgccgtgagcagcagcagcaccagaggcaccagcgccacacacagccaccacagcagccacaccacctctgccgcccacagcagatccggcagcgtgtcccagagagtgaccagcagccagaccgtgtcccacggcgtgaacgagacaatctacaacaccaccctgaagtacggcgacgtcgtgggcgtgaataccaccaagtacccctacagagtgtgcagcatggcccagggcaccgacctgatcagattcgagcggaacatcgtgtgcaccagcatgaagcccatcaacgaggacctggacgagggcatcatggtggtgtacaagagaaacatcgtggcccacaccttcaaagtgcgggtgtaccagaaggtgctgaccttccggcggagctacgcctacatccacaccacatacctgctgggcagcaacaccgagtacgtggcccctcccatgtgggagatccaccacatcaacagccacagccagtgctacagcagctacagccgcgtgatcgccggcacagtgttcgtggcctaccaccgggacagctacgagaacaagaccatgcagctgatgcccgacgactacagcaacacccacagcaccagatacgtgaccgtgaaggaccagtggcacagcagaggcagcacctggctgtaccgggagacatgcaacctgaactgcatggtcaccatcaccaccgccagaagcaagtacccttaccacttcttcgccacctccaccggcgacgtggtggacatcagccccttctacaacggcaccaaccggaacgccagctacttcggcgagaacgccgacaagttcttcatcttccccaactacaccatcgtgtccgacttcggcagacccaacagcgctctggaaacccacagactggtggcctttctggaacgggccgacagcgtgatcagctgggacatccaggacgagaagaacgtgacctgccagctgaccttctgggaggcctctgagagaaccatcagaagcgaggccgaggacagctaccacttcagcagcgccaagatgaccgccaccttcctgagcaagaaacaggaagtgaacatgagcgactccgccctggactgcgtgagggacgaggccatcaacaagctgcagcagatcttcaacaccagctacaaccagacctacgagaagtatggcaatgtgtccgtgttcgagacaacaggcggcctggtggtgttctggcagggcatcaagcagaaaagcctggtggagctggaacggctcgccaaccggtccagcctgaacctgacccacaaccggaccaagcggagcaccgacggcaacaacgcaacccacctgtccaacatggaaagcgtgcacaacctggtgtacgcacagctgcagttcacctacgacaccctgcggggctacatcaacagagccctggcccagatcgccgaggcttggtgcgtggaccagcggcggaccctggaagtgttcaaagagctgtccaagatcaaccccagcgccatcctgagcgccatctacaacaagcctatcgccgccagattcatgggcgacgtgctgggcctggccagctgcgtgaccatcaaccagaccagcgtgaaggtgctgcgggacatgaacgtgaaagagagcccaggccgctgctactccagacccgtggtcatcttcaacttcgccaacagctcctacgtgcagtacggccagctgggcgaggacaacgagatcctgctggggaaccaccggaccgaggaatgccagctgcccagcctgaagatctttatcgccggcaacagcgcctacgagtatgtggactacctgttcaagcggatgatcgacctgagcagcatctccaccgtggacagcatgatcgccctggacatcgaccccctggaaaacaccgacttccgggtgctggaactgtacagccagaaagagctgcggagcagcaacgtgttcgacctggaagagatcatgcgggagttcaacagctacaagcagcgcgtgaaatacgtggaggacaaggtggtggaccccctgcctccttacctgaagggcctggacgacctgatgagcggactgggcgctgccggaaaagccgtgggagtggccattggagctgtgggcggagctgtggcctctgtcgtggaaggcgtcgccacctttctgaagaaccccttcggcgccttcaccatcatcctggtggccattgccgtcgtgatcatcacctacctgatctacacccggcagcggagactgtgtacccagcccctgcagaacctgttcccctacctggtgtccgccgatggcaccacagtgaccagcggctccaccaaggataccagcctgcaggccccacccagctacgaagagagcgtgtacaacagcggcagaaagggccctggccctcccagctctgatgccagcacagccgcccctccctacaccaacgagcaggcctaccagatgctgctggccctggctagactggatgccgagcagagggcccagcagaacggcaccgacagcctggatggcagaaccggcacccaggacaagggccagaagcccaacctgctggaccggctgcggcaccggaagaacggctaccggcacctgaaggacagcgacgaggaagagaacgtctgataa

SEQ ID NO: 2 - protein, 907 aa, Cytomegalovirus sp.
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKAVGVAIGAVGGAVASVVEGVATFLKNPFGAFTIILVAIAVVIITYLIYTRQRRLCTQPLQNLFPYLVSADGTTVTSGSTKDTSLQAPPSYEESVYNSGRKGPGPPSSDASTAAPPYTNEQAYQMLLALARLDAEQRAQQNGTDSLDGRTGTQDKGQKPNLLDRLRHRKNGYRHLKDSDEEENV

SEQ ID NO: 3 - DNA, 2256 bp, Cytomegalovirus sp.
  source 1..2256: mol_type = genomic DNA
atggaaagccggatctggtgcctggtcgtgtgcgtgaacctgtgcatcgtgtgcctgggagccgccgtgagcagcagcagcaccagaggcaccagcgccacacacagccaccacagcagccacaccacctctgccgcccacagcagatccggcagcgtgtcccagagagtgaccagcagccagaccgtgtcccacggcgtgaacgagacaatctacaacaccaccctgaagtacggcgacgtcgtgggcgtgaataccaccaagtacccctacagagtgtgcagcatggcccagggcaccgacctgatcagattcgagcggaacatcgtgtgcaccagcatgaagcccatcaacgaggacctggacgagggcatcatggtggtgtacaagagaaacatcgtggcccacaccttcaaagtgcgggtgtaccagaaggtgctgaccttccggcggagctacgcctacatccacaccacatacctgctgggcagcaacaccgagtacgtggcccctcccatgtgggagatccaccacatcaacagccacagccagtgctacagcagctacagccgcgtgatcgccggcacagtgttcgtggcctaccaccgggacagctacgagaacaagaccatgcagctgatgcccgacgactacagcaacacccacagcaccagatacgtgaccgtgaaggaccagtggcacagcagaggcagcacctggctgtaccgggagacatgcaacctgaactgcatggtcaccatcaccaccgccagaagcaagtacccttaccacttcttcgccacctccaccggcgacgtggtggacatcagccccttctacaacggcaccaaccggaacgccagctacttcggcgagaacgccgacaagttcttcatcttccccaactacaccatcgtgtccgacttcggcagacccaacagcgctctggaaacccacagactggtggcctttctggaacgggccgacagcgtgatcagctgggacatccaggacgagaagaacgtgacctgccagctgaccttctgggaggcctctgagagaaccatcagaagcgaggccgaggacagctaccacttcagcagcgccaagatgaccgccaccttcctgagcaagaaacaggaagtgaacatgagcgactccgccctggactgcgtgagggacgaggccatcaacaagctgcagcagatcttcaacaccagctacaaccagacctacgagaagtatggcaatgtgtccgtgttcgagacaacaggcggcctggtggtgttctggcagggcatcaagcagaaaagcctggtggagctggaacggctcgccaaccggtccagcctgaacctgacccacaaccggaccaagcggagcaccgacggcaacaacgcaacccacctgtccaacatggaaagcgtgcacaacctggtgtacgcacagctgcagttcacctacgacaccctgcggggctacatcaacagagccctggcccagatcgccgaggcttggtgcgtggaccagcggcggaccctggaagtgttcaaagagctgtccaagatcaaccccagcgccatcctgagcgccatctacaacaagcctatcgccgccagattcatgggcgacgtgctgggcctggccagctgcgtgaccatcaaccagaccagcgtgaaggtgctgcgggacatgaacgtgaaagagagcccaggccgctgctactccagacccgtggtcatcttcaacttcgccaacagctcctacgtgcagtacggccagctgggcgaggacaacgagatcctgctggggaaccaccggaccgaggaatgccagctgcccagcctgaagatctttatcgccggcaacagcgcctacgagtatgtggactacctgttcaagcggatgatcgacctgagcagcatctccaccgtggacagcatgatcgccctggacatcgaccccctggaaaacaccgacttccgggtgctggaactgtacagccagaaagagctgcggagcagcaacgtgttcgacctggaagagatcatgcgggagttcaacagctacaagcagcgcgtgaaatacgtggaggacaaggtggtggaccccctgcctccttacctgaagggcctggacgacctgatgagcggactgggcgctgccggaaaagccgtgggagtggccattggagctgtgggcggagctgtggcctctgtcgtggaaggcgtcgccacctttctgaagaactgataa

SEQ ID NO: 4 - protein, 750 aa, Cytomegalovirus sp.
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKAVGVAIGAVGGAVASVVEGVATFLKN

SEQ ID NO: 5 - DNA, 2082 bp, Cytomegalovirus sp.
  source 1..2082: mol_type = genomic DNA
atggaaagccggatctggtgcctggtcgtgtgcgtgaacctgtgcatcgtgtgcctgggagccgccgtgagcagcagcagcaccagaggcaccagcgccacacacagccaccacagcagccacaccacctctgccgcccacagcagatccggcagcgtgtcccagagagtgaccagcagccagaccgtgtcccacggcgtgaacgagacaatctacaacaccaccctgaagtacggcgacgtcgtgggcgtgaataccaccaagtacccctacagagtgtgcagcatggcccagggcaccgacctgatcagattcgagcggaacatcgtgtgcaccagcatgaagcccatcaacgaggacctggacgagggcatcatggtggtgtacaagagaaacatcgtggcccacaccttcaaagtgcgggtgtaccagaaggtgctgaccttccggcggagctacgcctacatccacaccacatacctgctgggcagcaacaccgagtacgtggcccctcccatgtgggagatccaccacatcaacagccacagccagtgctacagcagctacagccgcgtgatcgccggcacagtgttcgtggcctaccaccgggacagctacgagaacaagaccatgcagctgatgcccgacgactacagcaacacccacagcaccagatacgtgaccgtgaaggaccagtggcacagcagaggcagcacctggctgtaccgggagacatgcaacctgaactgcatggtcaccatcaccaccgccagaagcaagtacccttaccacttcttcgccacctccaccggcgacgtggtggacatcagccccttctacaacggcaccaaccggaacgccagctacttcggcgagaacgccgacaagttcttcatcttccccaactacaccatcgtgtccgacttcggcagacccaacagcgctctggaaacccacagactggtggcctttctggaacgggccgacagcgtgatcagctgggacatccaggacgagaagaacgtgacctgccagctgaccttctgggaggcctctgagagaaccatcagaagcgaggccgaggacagctaccacttcagcagcgccaagatgaccgccaccttcctgagcaagaaacaggaagtgaacatgagcgactccgccctggactgcgtgagggacgaggccatcaacaagctgcagcagatcttcaacaccagctacaaccagacctacgagaagtatggcaatgtgtccgtgttcgagacaacaggcggcctggtggtgttctggcagggcatcaagcagaaaagcctggtggagctggaacggctcgccaaccggtccagcctgaacctgacccacaaccggaccaagcggagcaccgacggcaacaacgcaacccacctgtccaacatggaaagcgtgcacaacctggtgtacgcacagctgcagttcacctacgacaccctgcggggctacatcaacagagccctggcccagatcgccgaggcttggtgcgtggaccagcggcggaccctggaagtgttcaaagagctgtccaagatcaaccccagcgccatcctgagcgccatctacaacaagcctatcgccgccagattcatgggcgacgtgctgggcctggccagctgcgtgaccatcaaccagaccagcgtgaaggtgctgcgggacatgaacgtgaaagagagcccaggccgctgctactccagacccgtggtcatcttcaacttcgccaacagctcctacgtgcagtacggccagctgggcgaggacaacgagatcctgctggggaaccaccggaccgaggaatgccagctgcccagcctgaagatctttatcgccggcaacagcgcctacgagtatgtggactacctgttcaagcggatgatcgacctgagcagcatctccaccgtggacagcatgatcgccctggacatcgaccccctggaaaacaccgacttccgggtgctggaactgtacagccagaaagagctgcggagcagcaacgtgttcgacctggaagagatcatgcgggagttcaacagctacaagcagtgataa

SEQ ID NO: 6 - protein, 692 aa, Cytomegalovirus sp.
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQ

SEQ ID NO: 7 - DNA, 2232 bp, Cytomegalovirus sp.
  source 1..2232: mol_type = genomic DNA
atgaggcctggcctgccctcctacctgatcatcctggccgtgtgcctgttcagccacctgctgtccagcagatacggcgccgaggccgtgagcgagcccctggacaaggctttccacctgctgctgaacacctacggcagacccatccggtttctgcgggagaacaccacccagtgcacctacaacagcagcctgcggaacagcaccgtcgtgagagagaacgccatcagcttcaactttttccagagctacaaccagtactacgtgttccacatgcccagatgcctgtttgccggccctctggccgagcagttcctgaaccaggtggacctgaccgagacactggaaagataccagcagcggctgaatacctacgccctggtgtccaaggacctggccagctaccggtcctttagccagcagctcaaggctcaggatagcctcggcgagcagcctaccaccgtgccccctcccatcgacctgagcatcccccacgtgtggatgcctccccagaccacccctcacggctggaccgagagccacaccacctccggcctgcacagaccccacttcaaccagacctgcatcctgttcgacggccacgacctgctgtttagcaccgtgaccccctgcctgcaccagggcttctacctgatcgacgagctgagatacgtgaagatcaccctgaccgaggatttcttcgtggtcaccgtgtccatcgacgacgacacccccatgctgctgatcttcggccacctgcccagagtgctgttcaaggccccctaccagcgggacaacttcatcctgcggcagaccgagaagcacgagctgctggtgctggtcaagaaggaccagctgaaccggcactcctacctgaaggaccccgacttcctggacgccgccctggacttcaactacctggacctgagcgccctgctgagaaacagcttccacagatacgccgtggacgtgctgaagtccggacggtgccagatgctcgatcggcggaccgtggagatggccttcgcctatgccctcgccctgttcgccgctgccagacaggaagaggctggcgcccaggtgtcagtgcccagagccctggatagacaggccgccctgctgcagatccaggaattcatgatcacctgcctgagccagaccccccctagaaccaccctgctgctgtaccccacagccgtggatctggccaagagggccctgtggacccccaaccagatcaccgacatcacaagcctcgtgcggctcgtgtacatcctgagcaagcagaaccagcagcacctgatcccccagtgggccctgagacagatcgccgacttcgccctgaagctgcacaagacccatctggccagctttctgagcgccttcgccaggcaggaactgtacctgatgggcagcctggtccacagcatgctggtgcataccaccgagcggcgggagatcttcatcgtggagacaggcctgtgtagcctggccgagctgtcccactttacccagctgctggcccaccctcaccacgagtacctgagcgacctgtacaccccctgcagcagcagcggcagacgggaccacagcctggaacggctgaccagactgttccccgatgccaccgtgcctgctacagtgcctgccgccctgtccatcctgtccaccatgcagcccagcaccctggaaaccttccccgacctgttctgcctgcccctgggcgagagctttagcgccctgaccgtgtccgagcacgtgtcctacatcgtgaccaatcagtacctgatcaagggcatcagctaccccgtgtccaccacagtcgtgggccagagcctgatcatcacccagaccgacagccagaccaagtgcgagctgacccggaacatgcacaccacacacagcatcaccgtggccctgaacatcagcctggaaaactgcgctttctgtcagtctgccctgctggaatacgacgatacccagggcgtgatcaacatcatgtacatgcacgacagcgacgacgtgctgttcgccctggacccctacaacgaggtggtggtgtccagcccccggacccactacctgatgctgctgaagaacggcaccgtgctggaagtgaccgacgtggtggtggacgccaccgacagcagactgctgatgatgagcgtgtacgccctgagcgccatcatcggcatctacctgctgtaccggatgctgaaaacctgctgataa

SEQ ID NO: 8 - protein, 742 aa, Cytomegalovirus sp.
MRPGLPSYLIILAVCLFSHLLSSRYGAEAVSEPLDKAFHLLLNTYGRPIRFLRENTTQCTYNSSLRNSTVVRENAISFNFFQSYNQYYVFHMPRCLFAGPLAEQFLNQVDLTETLERYQQRLNTYALVSKDLASYRSFSQQLKAQDSLGEQPTTVPPPIDLSIPHVWMPPQTTPHGWTESHTTSGLHRPHFNQTCILFDGHDLLFSTVTPCLHQGFYLIDELRYVKITLTEDFFVVTVSIDDDTPMLLIFGHLPRVLFKAPYQRDNFILRQTEKHELLVLVKKDQLNRHSYLKDPDFLDAALDFNYLDLSALLRNSFHRYAVDVLKSGRCQMLDRRTVEMAFAYALALFAAARQEEAGAQVSVPRALDRQAALLQIQEFMITCLSQTPPRTTLLLYPTAVDLAKRALWTPNQITDITSLVRLVYILSKQNQQHLIPQWALRQIADFALKLHKTHLASFLSAFARQELYLMGSLVHSMLVHTTERREIFIVETGLCSLAELSHFTQLLAHPHHEYLSDLYTPCSSSGRRDHSLERLTRLFPDATVPATVPAALSILSTMQPSTLETFPDLFCLPLGESFSALTVSEHVSYIVTNQYLIKGISYPVSTTVVGQSLIITQTDSQTKCELTRNMHTTHSITVALNISLENCAFCQSALLEYDDTQGVINIMYMHDSDDVLFALDPYNEVVVSSPRTHYLMLLKNGTVLEVTDVVVDATDSRLLMMSVYALSAIIGIYLLYRMLKTC

SEQ ID NO: 9 - DNA, 2151 bp, Cytomegalovirus sp.
  source 1..2151: mol_type = genomic DNA
atgaggcctggcctgccctcctacctgatcatcctggccgtgtgcctgttcagccacctgctgtccagcagatacggcgccgaggccgtgagcgagcccctggacaaggctttccacctgctgctgaacacctacggcagacccatccggtttctgcgggagaacaccacccagtgcacctacaacagcagcctgcggaacagcaccgtcgtgagagagaacgccatcagcttcaactttttccagagctacaaccagtactacgtgttccacatgcccagatgcctgtttgccggccctctggccgagcagttcctgaaccaggtggacctgaccgagacactggaaagataccagcagcggctgaatacctacgccctggtgtccaaggacctggccagctaccggtcctttagccagcagctcaaggctcaggatagcctcggcgagcagcctaccaccgtgccccctcccatcgacctgagcatcccccacgtgtggatgcctccccagaccacccctcacggctggaccgagagccacaccacctccggcctgcacagaccccacttcaaccagacctgcatcctgttcgacggccacgacctgctgtttagcaccgtgaccccctgcctgcaccagggcttctacctgatcgacgagctgagatacgtgaagatcaccctgaccgaggatttcttcgtggtcaccgtgtccatcgacgacgacacccccatgctgctgatcttcggccacctgcccagagtgctgttcaaggccccctaccagcgggacaacttcatcctgcggcagaccgagaagcacgagctgctggtgctggtcaagaaggaccagctgaaccggcactcctacctgaaggaccccgacttcctggacgccgccctggacttcaactacctggacctgagcgccctgctgagaaacagcttccacagatacgccgtggacgtgctgaagtccggacggtgccagatgctcgatcggcggaccgtggagatggccttcgcctatgccctcgccctgttcgccgctgccagacaggaagaggctggcgcccaggtgtcagtgcccagagccctggatagacaggccgccctgctgcagatccaggaattcatgatcacctgcctgagccagaccccccctagaaccaccctgctgctgtaccccacagccgtggatctggccaagagggccctgtggacccccaaccagatcaccgacatcacaagcctcgtgcggctcgtgtacatcctgagcaagcagaaccagcagcacctgatcccccagtgggccctgagacagatcgccgacttcgccctgaagctgcacaagacccatctggccagctttctgagcgccttcgccaggcaggaactgtacctgatgggcagcctggtccacagcatgctggtgcataccaccgagcggcgggagatcttcatcgtggagacaggcctgtgtagcctggccgagctgtcccactttacccagctgctggcccaccctcaccacgagtacctgagcgacctgtacaccccctgcagcagcagcggcagacgggaccacagcctggaacggctgaccagactgttccccgatgccaccgtgcctgctacagtgcctgccgccctgtccatcctgtccaccatgcagcccagcaccctggaaaccttccccgacctgttctgcctgcccctgggcgagagctttagcgccctgaccgtgtccgagcacgtgtcctacatcgtgaccaatcagtacctgatcaagggcatcagctaccccgtgtccaccacagtcgtgggccagagcctgatcatcacccagaccgacagccagaccaagtgcgagctgacccggaacatgcacaccacacacagcatcaccgtggccctgaacatcagcctggaaaactgcgctttctgtcagtctgccctgctggaatacgacgatacccagggcgtgatcaacatcatgtacatgcacgacagcgacgacgtgctgttcgccctggacccctacaacgaggtggtggtgtccagcccccggacccactacctgatgctgctgaagaacggcaccgtgctggaagtgaccgacgtggtggtggacgccaccgactgataa

SEQ ID NO: 10 - protein, 715 aa, Cytomegalovirus sp.
MRPGLPSYLIILAVCLFSHLLSSRYGAEAVSEPLDKAFHLLLNTYGRPIRFLRENTTQCTYNSSLRNSTVVRENAISFNFFQSYNQYYVFHMPRCLFAGPLAEQFLNQVDLTETLERYQQRLNTYALVSKDLASYRSFSQQLKAQDSLGEQPTTVPPPIDLSIPHVWMPPQTTPHGWTESHTTSGLHRPHFNQTCILFDGHDLLFSTVTPCLHQGFYLIDELRYVKITLTEDFFVVTVSIDDDTPMLLIFGHLPRVLFKAPYQRDNFILRQTEKHELLVLVKKDQLNRHSYLKDPDFLDAALDFNYLDLSALLRNSFHRYAVDVLKSGRCQMLDRRTVEMAFAYALALFAAARQEEAGAQVSVPRALDRQAALLQIQEFMITCLSQTPPRTTLLLYPTAVDLAKRALWTPNQITDITSLVRLVYILSKQNQQHLIPQWALRQIADFALKLHKTHLASFLSAFARQELYLMGSLVHSMLVHTTERREIFIVETGLCSLAELSHFTQLLAHPHHEYLSDLYTPCSSSGRRDHSLERLTRLFPDATVPATVPAALSILSTMQPSTLETFPDLFCLPLGESFSALTVSEHVSYIVTNQYLIKGISYPVSTTVVGQSLIITQTDSQTKCELTRNMHTTHSITVALNISLENCAFCQSALLEYDDTQGVINIMYMHDSDDVLFALDPYNEVVVSSPRTHYLMLLKNGTVLEVTDVVVDATD

SEQ ID NO: 11 - DNA, 840 bp, Cytomegalovirus sp.
  source 1..840: mol_type = genomic DNA
atgtgcagaaggcccgactgcggcttcagcttcagccctggacccgtgatcctgctgtggtgctgcctgctgctgcctatcgtgtcctctgccgccgtgtctgtggcccctacagccgccgagaaggtgccagccgagtgccccgagctgaccagaagatgcctgctgggcgaggtgttcgagggcgacaagtacgagagctggctgcggcccctggtcaacgtgaccggcagagatggccccctgagccagctgatccggtacagacccgtgacccccgaggccgccaatagcgtgctgctggacgaggccttcctggataccctggccctgctgtacaacaaccccgaccagctgagagccctgctgaccctgctgtccagcgacaccgcccccagatggatgaccgtgatgcggggctacagcgagtgtggagatggcagccctgccgtgtacacctgcgtggacgacctgtgcagaggctacgacctgaccagactgagctacggccggtccatcttcacagagcacgtgctgggcttcgagctggtgccccccagcctgttcaacgtggtggtggccatccggaacgaggccaccagaaccaacagagccgtgcggctgcctgtgtctacagccgctgcacctgagggcatcacactgttctacggcctgtacaacgccgtgaaagagttctgcctccggcaccagctggatccccccctgctgagacacctggacaagtactacgccggcctgcccccagagctgaagcagaccagagtgaacctgcccgcccacagcagatatggccctcaggccgtggacgccagatgataa

SEQ ID NO: 12 - protein, 278 aa, Cytomegalovirus sp.
MCRRPDCGFSFSPGPVILLWCCLLLPIVSSAAVSVAPTAAEKVPAECPELTRRCLLGEVFEGDKYESWLRPLVNVTGRDGPLSQLIRYRPVTPEAANSVLLDEAFLDTLALLYNNPDQLRALLTLLSSDTAPRWMTVMRGYSECGDGSPAVYTCVDDLCRGYDLTRLSYGRSIFTEHVLGFELVPPSLFNVVVAIRNEATRTNRAVRLPVSTAAAPEGITLFYGLYNAVKEFCLRHQLDPPLLRHLDKYYAGLPPELKQTRVNLPAHSRYGPQAVDAR

SEQ ID NO: 13 - DNA, 1119 bp, Cytomegalovirus sp.
  source 1..1119: mol_type = genomic DNA
atggcccccagccacgtggacaaagtgaacacccggacttggagcgccagcatcgtgttcatggtgctgaccttcgtgaacgtgtccgtgcacctggtgctgtccaacttcccccacctgggctacccctgcgtgtactaccacgtggtggacttcgagcggctgaacatgagcgcctacaacgtgatgcacctgcacacccccatgctgtttctggacagcgtgcagctcgtgtgctacgccgtgttcatgcagctggtgtttctggccgtgaccatctactacctcgtgtgctggatcaagatcagcatgcggaaggacaagggcatgagcctgaaccagagcacccgggacatcagctacatgggcgacagcctgaccgccttcctgttcatcctgagcatggacaccttccagctgttcaccctgaccatgagcttccggctgcccagcatgatcgccttcatggccgccgtgcactttttctgtctgaccatcttcaacgtgtccatggtcacccagtaccggtcctacaagcggagcctgttcttcttctcccggctgcaccccaagctgaagggcaccgtgcagttccggaccctgatcgtgaacctggtggaggtggccctgggcttcaataccaccgtggtggctatggccctgtgctacggcttcggcaacaacttcttcgtgcggaccggccatatggtgctggccgtgttcgtggtgtacgccatcatcagcatcatctactttctgctgatcgaggccgtgttcttccagtacgtgaaggtgcagttcggctaccatctgggcgcctttttcggcctgtgcggcctgatctaccccatcgtgcagtacgacaccttcctgagcaacgagtaccggaccggcatcagctggtccttcggaatgctgttcttcatctgggccatgttcaccacctgcagagccgtgcggtacttcagaggcagaggcagcggctccgtgaagtaccaggccctggccacagcctctggcgaagaggtggccgccctgagccaccacgacagcctggaaagcagacggctgcgggaggaagaggacgacgacgacgaggacttcgaggacgcctgataa

SEQ ID NO: 14 - protein, 371 aa, Cytomegalovirus sp.
MAPSHVDKVNTRTWSASIVFMVLTFVNVSVHLVLSNFPHLGYPCVYYHVVDFERLNMSAYNVMHLHTPMLFLDSVQLVCYAVFMQLVFLAVTIYYLVCWIKISMRKDKGMSLNQSTRDISYMGDSLTAFLFILSMDTFQLFTLTMSFRLPSMIAFMAAVHFFCLTIFNVSMVTQYRSYKRSLFFFSRLHPKLKGTVQFRTLIVNLVEVALGFNTTVVAMALCYGFGNNFFVRTGHMVLAVFVVYAIISIIYFLLIEAVFFQYVKVQFGYHLGAFFGLCGLIYPIVQYDTFLSNEYRTGISWSFGMLFFIWAMFTTCRAVRYFRGRGSGSVKYQALATASGEEVAALSHHDSLESRRLREEEDDDDEDFEDA

SEQ ID NO: 15 - DNA, 411 bp, Cytomegalovirus sp.
  source 1..411: mol_type = genomic DNA
atggaatggaacaccctggtcctgggcctgctggtgctgtctgtcgtggccagcagcaacaacacatccacagccagcacccctagacctagcagcagcacccacgccagcactaccgtgaaggctaccaccgtggccaccacaagcaccaccactgctaccagcaccagctccaccacctctgccaagcctggctctaccacacacgaccccaacgtgatgaggccccacgcccacaacgacttctacaacgctcactgcaccagccacatgtacgagctgtccctgagcagctttgccgcctggtggaccatgctgaacgccctgatcctgatgggcgccttctgcatcgtgctgcggcactgctgcttccagaacttcaccgccaccaccaccaagggctactgataa

SEQ ID NO: 16 - protein, 135 aa, Cytomegalovirus sp.
MEWNTLVLGLLVLSVVASSNNTSTASTPRPSSSTHASTTVKATTVATTSTTTATSTSSTTSAKPGSTTHDPNVMRPHAHNDFYNAHCTSHMYELSLSSFAAWWTMLNALILMGAFCIVLRHCCFQNFTATTTKGY

SEQ ID NO: 17 - DNA, 1422 bp, Cytomegalovirus sp.
  source 1..1422: mol_type = genomic DNA
atgggcaagaaagaaatgatcatggtcaagggcatccccaagatcatgctgctgattagcatcacctttctgctgctgtccctgatcaactgcaacgtgctggtcaacagccggggcaccagaagatcctggccctacaccgtgctgtcctaccggggcaaagagatcctgaagaagcagaaagaggacatcctgaagcggctgatgagcaccagcagcgacggctaccggttcctgatgtaccccagccagcagaaattccacgccatcgtgatcagcatggacaagttcccccaggactacatcctggccggacccatccggaacgacagcatcacccacatgtggttcgacttctacagcacccagctgcggaagcccgccaaatacgtgtacagcgagtacaaccacaccgcccacaagatcaccctgaggcctcccccttgtggcaccgtgcccagcatgaactgcctgagcgagatgctgaacgtgtccaagcggaacgacaccggcgagaagggctgcggcaacttcaccaccttcaaccccatgttcttcaacgtgccccggtggaacaccaagctgtacatcggcagcaacaaagtgaacgtggacagccagaccatctactttctgggcctgaccgccctgctgctgagatacgcccagcggaactgcacccggtccttctacctggtcaacgccatgagccggaacctgttccgggtgcccaagtacatcaacggcaccaagctgaagaacaccatgcggaagctgaagcggaagcaggccctggtcaaagagcagccccagaagaagaacaagaagtcccagagcaccaccaccccctacctgagctacaccacctccaccgccttcaacgtgaccaccaacgtgacctacagcgccacagccgccgtgaccagagtggccacaagcaccaccggctaccggcccgacagcaactttatgaagtccatcatggccacccagctgagagatctggccacctgggtgtacaccaccctgcggtacagaaacgagcccttctgcaagcccgaccggaacagaaccgccgtgagcgagttcatgaagaatacccacgtgctgatcagaaacgagacaccctacaccatctacggcaccctggacatgagcagcctgtactacaacgagacaatgagcgtggagaacgagacagccagcgacaacaacgaaaccacccccacctcccccagcacccggttccagcggaccttcatcgaccccctgtgggactacctggacagcctgctgttcctggacaagatccggaacttcagcctgcagctgcccgcctacggcaatctgaccccccctgagcacagaagggccgccaacctgagcaccctgaacagcctgtggtggtggagccagtgataa

SEQ ID NO: 18 - protein, 472 aa, Cytomegalovirus sp.
MGKKEMIMVKGIPKIMLLISITFLLLSLINCNVLVNSRGTRRSWPYTVLSYRGKEILKKQKEDILKRLMSTSSDGYRFLMYPSQQKFHAIVISMDKFPQDYILAGPIRNDSITHMWFDFYSTQLRKPAKYVYSEYNHTAHKITLRPPPCGTVPSMNCLSEMLNVSKRNDTGEKGCGNFTTFNPMFFNVPRWNTKLYIGSNKVNVDSQTIYFLGLTALLLRYAQRNCTRSFYLVNAMSRNLFRVPKYINGTKLKNTMRKLKRKQALVKEQPQKKNKKSQSTTTPYLSYTTSTAFNVTTNVTYSATAAVTRVATSTTGYRPDSNFMKSIMATQLRDLATWVYTTLRYRNEPFCKPDRNRTAVSEFMKNTHVLIRNETPYTIYGTLDMSSLYYNETMSVENETASDNNETTPTSPSTRFQRTFIDPLWDYLDSLLFLDKIRNFSLQLPAYGNLTPPEHRRAANLSTLNSLWWWSQ

SEQ ID NO: 19 - DNA, 519 bp, Cytomegalovirus sp.
  source 1..519: mol_type = genomic DNA
atgagccccaaggacctgacccccttcctgacaaccctgtggctgctcctgggccatagcagagtgcctagagtgcgggccgaggaatgctgcgagttcatcaacgtgaaccacccccccgagcggtgctacgacttcaagatgtgcaaccggttcaccgtggccctgagatgccccgacggcgaagtgtgctacagccccgagaaaaccgccgagatccggggcatcgtgaccaccatgacccacagcctgacccggcaggtggtgcacaacaagctgaccagctgcaactacaaccccctgtacctggaagccgacggccggatcagatgcggcaaagtgaacgacaaggcccagtacctgctgggagccgccggaagcgtgccctaccggtggatcaacctggaatacgacaagatcacccggatcgtgggcctggaccagtacctggaaagcgtgaagaagcacaagcggctggacgtgtgcagagccaagatgggctacatgctgcagtgataa

SEQ ID NO: 20 - protein, 171 aa, Cytomegalovirus sp.
MSPKDLTPFLTTLWLLLGHSRVPRVRAEECCEFINVNHPPERCYDFKMCNRFTVALRCPDGEVCYSPEKTAEIRGIVTTMTHSLTRQVVHNKLTSCNYNPLYLEADGRIRCGKVNDKAQYLLGAAGSVPYRWINLEYDKITRIVGLDQYLESVKKHKRLDVCRAKMGYMLQ

SEQ ID NO: 21 - DNA, 648 bp, Cytomegalovirus sp.
  source 1..648: mol_type = genomic DNA
atgctgcggctgctgctgagacaccacttccactgcctgctgctgtgtgccgtgtgggccaccccttgtctggccagcccttggagcaccctgaccgccaaccagaaccctagccccccttggtccaagctgacctacagcaagccccacgacgccgccaccttctactgcccctttctgtaccccagccctcccagaagccccctgcagttcagcggcttccagagagtgtccaccggccctgagtgccggaacgagacactgtacctgctgtacaaccgggagggccagacactggtggagcggagcagcacctgggtgaaaaaagtgatctggtatctgagcggccggaaccagaccatcctgcagcggatgcccagaaccgccagcaagcccagcgacggcaacgtgcagatcagcgtggaggacgccaaaatcttcggcgcccacatggtgcccaagcagaccaagctgctgagattcgtggtcaacgacggcaccagatatcagatgtgcgtgatgaagctggaaagctgggcccacgtgttccgggactactccgtgagcttccaggtccggctgaccttcaccgaggccaacaaccagacctacaccttctgcacccaccccaacctgatcgtgtgataa

SEQ ID NO: 22 - protein, 214 aa, Cytomegalovirus sp.
MLRLLLRHHFHCLLLCAVWATPCLASPWSTLTANQNPSPPWSKLTYSKPHDAATFYCPFLYPSPPRSPLQFSGFQRVSTGPECRNETLYLLYNREGQTLVERSSTWVKKVIWYLSGRNQTILQRMPRTASKPSDGNVQISVEDAKIFGAHMVPKQTKLLRFVVNDGTRYQMCVMKLESWAHVFRDYSVSFQVRLTFTEANNQTYTFCTHPNLIV

SEQ ID NO: 23 - DNA, 393 bp, Cytomegalovirus sp.
  source 1..393: mol_type = genomic DNA
atgcggctgtgcagagtgtggctgtccgtgtgcctgtgtgccgtggtgctgggccagtgccagagagagacagccgagaagaacgactactaccgggtgccccactactgggatgcctgcagcagagccctgcccgaccagacccggtacaaatacgtggagcagctcgtggacctgaccctgaactaccactacgacgccagccacggcctggacaacttcgacgtgctgaagcggatcaacgtgaccgaggtgtccctgctgatcagcgacttccggcggcagaacagaagaggcggcaccaacaagcggaccaccttcaacgccgctggctctctggcccctcacgccagatccctggaattcagcgtgcggctgttcgccaactgataa

SEQ ID NO: 24 - protein, 129 aa, Cytomegalovirus sp.
MRLCRVWLSVCLCAVVLGQCQRETAEKNDYYRVPHYWDACSRALPDQTRYKYVEQLVDLTLNYHYDASHGLDNFDVLKRINVTEVSLLISDFRRQNRRGGTNKRTTFNAAGSLAPHARSLEFSVRLFAN

SEQ ID NO: 25 - DNA, 2371 bp, unidentified
  misc_feature 1..2371: note = Parvovirus
  source 1..2371: mol_type = unassigned DNA
acgcgtacaaaacaaaatgtctaagaaatctggtaaatggtgggaatctgatgataaatttgctaaggctgtttaccaacaatttgttgaattttacgaaaaggttactggtactgatttggaattgattcaaattttgaaggatcattacaacatttctttggataatccattggaaaatccatcttcattgtttgatttggttgctagaattaagaacaacttgaagaactctccagatttgtattctcatcatttccaatctcatggtcaattgtctgatcatccacatgctttatcttcatcttcatctcatgctgaaccaagaggtgaaaatgctgttttatcttctgaagatttgcataaaccaggtcaagtttctgttcaattgccaggtactaattacgttggtccaggtaatgaattgcaagctggtccaccacaatctgctgttgattctgctgctagaattcatgatttcagatactctcaattggctaagttgggtattaatccatatactcattggactgttgctgatgaagaattgttgaagaacattaagaatgaaactggttttcaagctcaagttgttaaagattacttcactttgaaaggtgctgctgctccagttgctcattttcaaggttctttgccagaagttccagcttataacgcttctgaaaaatatccatctatgacatctgttaattctgctgaagcatctactggtgcaggtggaggtggttctaattctgttaaatctatgtggtctgaaggtgctactttttctgctaattcagttacttgtactttctctagacaattcttgattccatatgatccagaacatcattacaaagttttttcaccagctgcttcatcttgtcataatgcttcaggtaaagaagctaaggtttgtactatttctccaattatgggttattctactccttggagatacttggattttaatgctttgaacttgtttttttctccattggaatttcaacatttgattgaaaactacggttctattgctccagatgctttgactgttactatttctgaaattgctgttaaggatgttactgataaaacaggtggtggtgttcaagttactgattctactactggtagattgtgcatgttggttgatcatgaatacaaatacccatacgttttgggtcaaggtcaagatactttggctccagaattgccaatttgggtttattttccaccacaatacgcttatttgactgttggtgatgttaatactcaaggtatttctggtgattctaaaaagttggcttctgaagaatctgctttttacgttttggaacattcttcttttcaattgttgggtactggtggtactgcttctatgtcttacaaatttccaccagttccacctgaaaatttggaaggttgttctcaacatttttacgaaatgtacaatccattgtatggttctagattgggtgttccagatactttgggtggtgatccaaaatttagatctttgactcatgaagatcatgctattcaaccacaaaatttcatgccaggtccattggttaattctgtttctactaaagaaggtgattcttctaatacaggtgctggtaaagcattgactggtttgtctactggtacttctcaaaacactagaatttctttaagaccaggtccagtttcacaaccatatcatcattgggatactgataagtacgttactggtattaatgctatttcacatggtcaaactacttatggtaatgctgaagataaagaatatcaacaaggtgttggtagatttccaaacgaaaaagaacaattgaaacaattgcaaggtttgaatatgcatacttactttccaaacaaaggtactcaacaatacactgatcaaattgaaagaccattgatggttggttctgtttggaatagaagagctttgcattatgaatctcaattgtggtctaagattccaaatttagatgattctttcaagactcaatttgctgctttgggtggttggggtttgcatcaacctccaccacaaattttcttgaagattttgccacaatctggtccaattggtggtattaaatctatgggtattactactttggttcaatatgctgttggtattatgactgttacaatgacttttaagttgggtccaagaaaagctacaggtagatggaatccacaaccaggtgtttatccaccacatgctgctggtcatttgccttacgttttgtatgatccaactgctactgatgctaaacaacatcatagacatggttatgaaaaacctgaagaattgtggactgctaaatctagagttcatccattgtaatgagtcgac

SEQ ID NO: 26 - DNA, 1692 bp, unidentified
  misc_feature 1..1692: note = Parvovirus
  source 1..1692: mol_type = unassigned DNA
cctaggacaaaacaaaatgacatctgttaattctgctgaagcatctactggtgcaggtggaggtggttctaattctgttaaatctatgtggtctgaaggtgctactttttctgctaattcagttacttgtactttctctagacaattcttgattccatatgatccagaacatcattacaaagttttttcaccagctgcttcatcttgtcataatgcttcaggtaaagaagctaaggtttgtactatttctccaattatgggttattctactccttggagatacttggattttaatgctttgaacttgtttttttctccattggaatttcaacatttgattgaaaactacggttctattgctccagatgctttgactgttactatttctgaaattgctgttaaggatgttactgataaaacaggtggtggtgttcaagttactgattctactactggtagattgtgcatgttggttgatcatgaatacaaatacccatacgttttgggtcaaggtcaagatactttggctccagaattgccaatttgggtttattttccaccacaatacgcttatttgactgttggtgatgttaatactcaaggtatttctggtgattctaaaaagttggcttctgaagaatctgctttttacgttttggaacattcttcttttcaattgttgggtactggtggtactgcttctatgtcttacaaatttccaccagttccacctgaaaatttggaaggttgttctcaacatttttacgaaatgtacaatccattgtatggttctagattgggtgttccagatactttgggtggtgatccaaaatttagatctttgactcatgaagatcatgctattcaaccacaaaatttcatgccaggtccattggttaattctgtttctactaaagaaggtgattcttctaatacaggtgctggtaaagcattgactggtttgtctactggtacttctcaaaacactagaatttctttaagaccaggtccagtttcacaaccatatcatcattgggatactgataagtacgttactggtattaatgctatttcacatggtcaaactacttatggtaatgctgaagataaagaatatcaacaaggtgttggtagatttccaaacgaaaaagaacaattgaaacaattgcaaggtttgaatatgcatacttactttccaaacaaaggtactcaacaatacactgatcaaattgaaagaccattgatggttggttctgtttggaatagaagagctttgcattatgaatctcaattgtggtctaagattccaaatttagatgattctttcaagactcaatttgctgctttgggtggttggggtttgcatcaacctccaccacaaattttcttgaagattttgccacaatctggtccaattggtggtattaaatctatgggtattactactttggttcaatatgctgttggtattatgactgttacaatgacttttaagttgggtccaagaaaagctacaggtagatggaatccacaaccaggtgtttatccaccacatgctgctggtcatttgccttacgttttgtatgatccaactgctactgatgctaaacaacatcatagacatggttatgaaaaacctgaagaattgtggactgctaaatctagagttcatccattgtaatgagcggccgc

(19) United States
(12) Patent Application Publication
(10) Pub. No.: US 2024/0082329 A1
(43) Pub. Date: Mar. 14, 2024

(54) IMMUNOGENIC COMBINATION COMPOSITIONS AND USES THEREOF

(71) Applicant: GLAXOSMITHKLINE BIOLOGICALS SA, RIXENSART (BE)

(72) Inventors: Andrew GEALL, Littleton, MA (US); Ethan SETTEMBRE, Lexington, MA (US)

(73) Assignee: GLAXOSMITHKLINE BIOLOGICALS SA, RIXENSART (BE)

(21) Appl. No.: 18/463,099

(22) Filed: Sep. 7, 2023

Related U.S. Application Data
(63) Continuation of application No. 14/130,899, filed on Apr. 7, 2014.
(60) Provisional application No. 61/505,093, filed on Jul. 6, 2011.

(51) Int. Cl. A61K 35/763; A61K 35/76; A61K 39/12; A61K 39/23; A61K 39/245

(52) U.S. Cl. CPC A61K 35/763; A61K 35/76; A61K 39/12; A61K 39/23; A61K 39/245; A61K 2039/5258

(57) ABSTRACT

This invention generally relates to immunogenic compositions that comprise an RNA component and a polypeptide component. Immunogenic compositions that deliver antigens in two different forms—a first antigen from a pathogen, in RNA-coded form; and a second antigen from a different pathogen, in polypeptide form—are effective in inducing immune response to both pathogens.

### RELATED APPLICATION

This application is a Continuation of copending application Ser. No. 14/130,899 filed on Apr. 7, 2014, which is a U.S. National Phase of International Application No. PCT/US2012/045847, filed Jul. 6, 2012, and published in English, which claims priority of U.S. Provisional Application No. 61/505,093 filed on Jul. 6, 2011. The entire contents of the foregoing applications are hereby incorporated by reference.

### REFERENCE TO ELECTRONIC SEQUENCE LISTING

The application contains a Sequence Listing which has been submitted electronically in .XML format and is hereby incorporated by reference in its entirety. Said .XML copy, created on Sep. 22, 2023, is named “2801-0320PUS2.xml” and is 50,606 bytes in size. The sequence listing contained in this .XML file is part of the specification and is hereby incorporated by reference herein in its entirety.

### BACKGROUND OF THE INVENTION

Combination vaccines merge antigens that prevent different diseases or that protect against multiple strains of infectious agents causing the same disease into a single product. Thus, they reduce the number of injections required to prevent some diseases. Potential advantages of combination vaccines include a) reducing the cost of stocking and administering separate vaccines, b) reducing the cost for extra health-care visits, and c) facilitating the addition of new vaccines into immunization programs.

Nucleic acid based vaccines are an attractive approach to vaccination. For example, intramuscular (IM) immunization of plasmid DNA encoding an antigen can induce cellular and humoral immune responses and protect against challenge. DNA vaccines offer certain advantages over traditional vaccines using protein antigens, or attenuated pathogens. For example, as compared to protein vaccines, DNA vaccines can be more effective in producing a properly folded antigen in its native conformation, and in generating a cellular immune response. DNA vaccines also do not have some of the safety problems associated with killed or attenuated pathogens. For example, a killed viral preparation may contain residual live viruses, and an attenuated virus may mutate and revert to a pathogenic phenotype. DNA vaccines are generally effective in generating cell mediated immunity (such as interferon-7 secreting antigen-specific T-cells and antigen-specific cytotoxic T-cells), but less effective in generating antibodies against the encoded and expressed antigen.

WO 97/28818 discloses a vaccine that delivers a nucleic acid and a protein antigen to antigen presenting cells. The nucleic acid may encode the same protein as the protein antigen. The nucleic acid and protein are “complexed,” e.g., by covalent conjugation. The complex may be formulated as a synthetic virus-like particle. It is also suggested that liposomal systems may be used.

U.S. Pat. No. 7,604,803 discloses the co-delivery of nucleic acid and its encoded protein to the same cell using a liposomal system. The DNA molecule and its encoded protein are entrapped within the same liposomal vehicle, such that the two entities arrive at antigen-presenting cells together, resulting in the processing and presentation of the protein form of the antigen, together with the expression of the DNA-encoded form of the antigen in the same cell.

WO 2009/074861 discloses a vaccine comprising (i) a nucleic acid sequence encoding at least one influenza virus antigen coated onto carrier particles, and (ii) an assistor protein for sequential or concomitant administration. The assistor protein and the antigen encoded by the nucleic acid molecule share at least one common epitope.

It is known that non-coding plasmid DNA has an immuno-adjuvant action when co-entrapped with peptides in liposomal vesicles (Gursel, M. et al. Vaccine (1999) 17: 1376-1383) and that DNA with CpG motifs has an adjuvant effect on naked DNA and peptide vaccines (Klinman, D. M. et al. Vaccine (1999) 17: 19-25).

Concerns have been raised regarding the safety of DNA-based vaccines. The introduced DNA molecules could potentially integrate into the host genome or, due to their distribution to various tissues, could lead to undesirable sustained expression of antigens. In addition, certain DNA viruses have also been used to deliver DNA molecules. Because of their infectious properties, such viruses achieve a very high transfection rate. The viruses used are genetically modified to prevent the formation of functional infectious particles in the transfected cell. Despite these precautions, however, it is not possible to rule out the risk of uncontrolled propagation of the introduced gene and viral genes, for example due to potential recombination events. This also entails the risk of the DNA being inserted into an intact gene of the host cell's genome by e.g. recombination, with the consequence that the host gene may be mutated and thus completely or partially inactivated or may give rise to misinformation. In other words, synthesis of a host gene product which is vital to the cell may be completely suppressed or, alternatively, a modified or incorrect gene product is expressed.

RNA molecules encoding an antigen or a derivative thereof may also be used as vaccines. RNA vaccines offer certain advantages as compared to DNA vaccines. However, compared with DNA-based vaccines, relatively minor attention has been given to RNA-based vaccines. RNAs are highly susceptible to degradation by nucleases when administered as a therapeutic or vaccine. See, e.g., Vajdy, M., et al., Mucosal adjuvants and delivery systems for protein-, DNA-and RNA-based vaccines, Immunol Cell Biol, 2004. 82(6): p. 617-27.

Toll-like receptors (TLRs) are a group of pattern recognition receptors which bind to pathogen-associated molecular patterns (PAMPS) from bacteria, fungi, protozoa and viruses, and act as a first line of defense against invading pathogens. Many TLRs have been identified in humans, mice, and other mammalian species. DNA molecules (such as bacterial or viral DNA) are recognized by TLR9, whereas RNA molecules (such as single stranded viral RNA) are recognized by TLR7 or TLR8.

T-cells and B-cells recognize antigens in different ways. T-cells recognize peptide fragments of proteins that are embedded in class-II or class-I MHC molecules at the surface of cells, whereas B-cells recognize surface features of an unprocessed antigen, via immunoglobulin-like cell surface receptors. The difference in antigen recognition mechanisms of T-cells and B-cells are reflected in the different natures of their epitopes. Thus, whereas B-cells recognize surface features of an antigen or a pathogen, T-cell epitopes (which comprise peptides of about 8-12 amino acids in length) can be “internal” as well as “surface” when viewed in the context of the three-dimensional structure of the antigen. Accordingly, a B-cell epitope is preferably exposed on the surface of the antigen or pathogen, and can be linear or conformational, whereas a T-cell epitope is typically linear but is not required to be available or on the surface of the antigen.

U.S. Pat. No. 7,862,829 discloses a method of producing an immune response by administering to an antigen and an alpha-virus-based adjuvant. The method is based on the discovery that alphavirus, a (+) ssRNA virus, can act as an adjuvant to enhance an immune response against an antigen, even though the antigen is not presented on or expressed by the virus. The alphavirus particles may be delivered by liposomal system.

There is a need to improve the efficacy of protein subunit vaccines and nucleic acid vaccines such as RNA vaccines.

### SUMMARY OF THE INVENTION

This invention generally relates to immunogenic compositions that comprise an RNA component and a polypeptide component. The immunogenic compositions deliver a combination of antigenic epitopes in two different forms—a first epitope from a pathogen, in RNA-coded form; and a second epitope from a different pathogen, in polypeptide form—and can induce an immune response to both pathogens (e.g., without the need for a separate adjuvant). A practical benefit of the immunogenic compositions described herein is that the total number of immunogenic compositions needed to be administered to the patient is reduced due to the combination of two or more antigens in a single immunogenic composition. This is especially beneficial for infants and children who receive a large number of routine vaccinations.

The invention also relates to methods for treating or preventing two or more infectious diseases, methods for inducing an immune response against two or more pathogens, or methods of vaccinating a subject, by co-delivery of an RNA molecule and a polypeptide molecule (co-administration).

In one aspect, the invention provides an immunogenic composition comprising (i) a first polypeptide antigen, and (ii) a self-replicating RNA molecule that encodes a second polypeptide antigen, wherein the first and second antigens are antigens from different pathogens. In some embodiments, the first polypeptide antigen is a Cytomegalovirus (CMV) antigen. In some embodiments, the second polypeptide antigen is a Parvovirus antigen. The second polypeptide antigen can be in the form of a virus-like particle (CLP).

In some embodiments, the first polypeptide antigen is a soluble polypeptide, and the second polypeptide antigen is a soluble or membrane anchored polypeptide.

In some embodiments, the self-replicating RNA is an alphavirus-derived RNA replicon. The self-replicating RNA molecule may comprise one or more modified nucleotides.

In some embodiments, the immunogenic composition further comprises a cationic lipid, a liposome, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, a polycationic peptide or a cationic nanoemulsion.

In some embodiments, the RNA molecule is encapsulated in, bound to or adsorbed on a cationic lipid, a liposome, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, a polycationic peptide, a cationic nanoemulsion, or combinations thereof.

In some embodiments, the first and second polypeptide antigens are independently derived from a viral pathogen, a bacterial pathogen, a fungal pathogen, a protozoan pathogen, and a multi-cellular parasitic pathogen. The first polypeptide antigen and the second polypeptide antigen can both be viral antigens. In such instances, one viral antigen can be from CMV. In another instance, one viral antigen can be from Parvovirus. The parvovirus antigen can comprise an amino acid sequence selected from SEQ ID NOs:25-26. The viral antigen from CMV can be a gB antigen, a gH antigen, or a gL antigen. In some embodiments, the viral antigen from CMV can be a gH antigen or a gL antigen.

In some embodiments, the RNA molecule encodes a gH antigen and a gL antigen. In some embodiments, the immunogenic composition comprises a gH polypeptide antigen and gL polypeptide antigen.

In some embodiments, the immunogenic composition further comprises an adjuvant.

The invention also relates to immunogenic compositions that comprise (i) a Parvovirus polypeptide antigen, and (ii) a self-replicating RNA molecule that encodes a CMV polypeptide antigen.

The invention also relates to immunogenic compositions and a pharmaceutically acceptable carrier and/or a pharmaceutically acceptable vehicle.

The invention also relates to methods for treating or preventing an infectious disease. In some embodiments, a therapeutically effective amount of an immunogenic composition is administered to a subject.

The invention also relates to methods for inducing an immune response in a subject. In some embodiments, a therapeutically effective amount of an immunogenic composition is administered to the subject.

The invention also relates to methods of vaccinating a subject. In some embodiments, an immunogenic composition is administered to the subject.

### DETAILED DESCRIPTION OF THE INVENTION

Certain terms that are used to describe the invention in this are defined and explained herein in Section 7.

### 1. Overview

One particular advantage of an RNA vaccine is that RNA molecules are self-adjuvanting. For example, RNA molecules can induce the production of cytokines, which can enhance the host immune response to the protein antigen that is encoded by the RNA molecule.

Vaccination strategies that combine an RNA molecule and a polypeptide molecule (e.g., administering an immunogenic composition that has an RNA component and a protein component) provide several benefits. RNA molecules promote type 1 T helper responses (Th1, IFN-γ^{hi}, IL-4^{lo}), whereas protein molecules promote type 2 T helper responses. Thus, combining an RNA molecule and a polypeptide molecule can promote both T cell-mediated immunity as well as humoral immunity. In addition, RNA molecules may be delivered to cells using delivery systems such as liposomes or oil-in-water emulsions. Liposomes and oil-in-water emulsions are also known to have adjuvant activities. Thus, the adjuvant activity of the RNA together with adjuvant activity of the delivery system can act synergistically to enhance the immune response to one or both antigens.

In one aspect, the invention relates to immunogenic compositions that comprise an RNA component from a first pathogen and a polypeptide component from a second pathogen. Immunogenic compositions that deliver antigenic epitopes in two different forms—a first epitope from one pathogen, in RNA-coded form; and a second epitope from a different pathogen, in polypeptide form—can enhance the immune response to one or both pathogens.

As described herein, the inventors have evaluated the efficacies of immunogenic compositions that comprise (i) a self-replicating RNA molecule that encodes a CMV antigen, and (ii) a Parvovirus polypeptide antigen. These studies demonstrated that co-administering an RNA molecule that encodes a CMV antigen, together with the Parvovirus antigen in polypeptide form, potentiated the immune response to the Parvovirus antigen, resulting in higher antibody titers as compared to administering the Parvovirus polypeptide molecule alone.

The immunogenic compositions described herein can be formulated as a vaccine to induce or enhance the host immune response to a pathogen, such as to induce protective immunity. Also provided herein are methods of using the immunogenic compositions of the invention to induce or enhance an immune response in a subject in need thereof

### 2. Immunogenic Compositions

In one aspect, the invention provides an immunogenic composition comprising: (i) a self-replicating RNA molecule that encodes a first polypeptide antigen; and (ii) a second polypeptide antigen; wherein said first and second polypeptide antigens are from different pathogens.

In certain embodiments, the RNA molecule may encode a first polypeptide antigen comprising a full-length protein from a pathogen (e.g., a viral protein), or an antigenic portion thereof, optionally fused with a tag sequence that may facilitate the expression, purification and/or detection of the protein. The second polypeptide antigen may be a recombinant protein comprising a full-length protein from a different pathogen, or an antigenic portion thereof, optionally fused with a tag sequence that may facilitate the production, purification or detection of the protein. The first polypeptide antigen, the second polypeptide antigen, or both, may comprise a mutation variant of a protein from a pathogen (e.g., a viral protein having amino acid substitution(s), addition(s), or deletion(s)).

In certain embodiments, the first polypeptide antigen is a soluble or membrane anchored polypeptide, and the second polypeptide antigen is a soluble polypeptide. For example, if the wild type viral protein is a transmembrane surface protein, the RNA molecule may comprise the full-length coding sequence to produce the first (membrane-anchored) antigen, while the transmembrane region of the viral protein may be deleted to produce the second polypeptide antigen (which is soluble).

In certain embodiments, the first antigen or the second antigen is a fusion polypeptide further comprising a third epitope. The third epitope may be from a different pathogen, or from a different antigen of the same pathogen.

The self-replicating RNA molecule that encodes a first polypeptide antigent can be in the form of a VRP. The second polypeptide antigen can be in the form of a VLP.

A. Antigens

Antigens suitable for inclusion in the immunogenic compositions described herein (either in polypeptide form or in RNA-encoded form) may be derived from any pathogen (e.g., a bacterial pathogen, a viral pathogen, a fungal pathogen, a protozoan pathogen, or a multi-cellular parasitic pathogen), allergen or tumor.

In certain embodiments, the first and/or second antigens are derived from a viral pathogen. Exemplary viral pathogens include, e.g., respiratory syncytial virus (RSV), hepatitis B virus (HBV), hepatitis C virus (HCV), Dengue virus, herpes simplex virus (HSV; e.g., HSV-I, HSV-II), molluscum contagiosum virus, vaccinia virus, variola virus, lentivirus, human immunodeficiency virus (HIV), human papilloma virus (HPV), cytomegalovirus (CMV), varicella zoster virus (VZV), rhinovirus, enterovirus, adenovirus, coronavirus (e.g., SARS), influenza virus (flu), para-influenza virus, mumps virus, measles virus, papovavirus, hepadnavirus, flavivirus, retrovirus, arenavirus (e.g., Lymphocytic Choriomeningitis Virus, Junin virus, Machupo virus, Guanarito virus, or Lassa virus), norovirus, yellow fever virus, rabies virus, Filovirus (e.g., Ebola virus or marbug virus), hepatitis C virus, hepatitis B virus, hepatitis A virus, Morbilliviruses (e.g., measles virus), Rubulaviruses (e.g., mumps virus), Rubiviruses (e.g., rubella virus), bovine viral diarrhea virus. For example, the antigen can be CMV glycoprotein gH, or gL; Parvovirus; HIV glycoprotein gp120 or gp140, HIV p55 gag, pol; or RSV-F antigen, etc.

In some embodiments, the first and/or second antigens are derived from a virus which infects fish, such as: infectious salmon anemia virus (ISAV), salmon pancreatic disease virus (SPDV), infectious pancreatic necrosis virus (IPNV), channel catfish virus (CCV), fish lymphocystis disease virus (FLDV), infectious hematopoietic necrosis virus (IHNV), koi herpesvirus, salmon picorna-like virus (also known as picorna-like virus of atlantic salmon), landlocked salmon virus (LSV), atlantic salmon rotavirus (ASR), trout strawberry disease virus (TSD), coho salmon tumor virus (CSTV), or viral hemorrhagic septicemia virus (VHSV).

In some embodiments the first and/or second antigens are derived from a parasite from the Plasmodium genus, such as P. falciparum, P. vivax, P. malariae or P. ovale. Thus the invention may be used for immunizing against malaria. In some embodiments the first and/or second antigens are derived from a parasite from the Caligidae family, particularly those from the Lepeophtheirus and Caligus genera e.g. sea lice such as Lepeophtheirus salmonis or Caligus rogercresseyi.

In certain embodiments, first and/or second antigens are derived from a bacterial pathogen. Exemplary bacterial pathogens include, e.g., Neisseria spp, including N. gonorrhea and N. meningitides; Streptococcus spp, including S. pneumoniae, S. pyogenes, S. agalactiae, S. mutans; Haemophilus spp, including H. influenzae type B, non typeable H. influenzae, H. ducreyi; Moraxella spp, including M. catarrhalis, also known as Branhamella catarrhalis; Bordetella spp, including B. pertussis, B. parapertussis and B. bronchiseptica; Mycobacterium spp., including M. tuberculosis, M. bovis, M. leprae, M. avium, M. paratuberculosis, M. smegmatis; Legionella spp, including L. pneumophila; Escherichia spp, including enterotoxic E. coli, enterohemorragic E. coli, enteropathogenic E. coli; Vibrio spp, including V. cholera, Shigella spp, including S. sonnei, S. dysenteriae, S. flexnerii; Yersinia spp, including Y. enterocolitica, Y. pestis, Y. pseudotuberculosis, Campylobacter spp, including C. jejuni and C. coli; Salmonella spp, including S. typhi, S. paratyphi, S. choleraesuis, S. enteritidis; Listeria spp., including L. monocytogenes; Helicobacter spp, including H pylori; Pseudomonas spp, including P. aeruginosa, Staphylococcus spp., including S. aureus, S. epidermidis; Enterococcus spp., including E. faecalis, E. faecium; Clostridium spp., including C. tetani, C. botulinum, C. difficile; Bacillus spp., including B. anthracis; Corynebacterium spp., including C. diphtheriae; Borrelia spp., including B. burgdorferi, B. garinii, B. afzelii, B. andersonii, B. hermsii; Ehrlichia spp., including E. equi and the agent of the Human Granulocytic Ehrlichiosis; Rickettsia spp, including R. rickettsii; Chlamydia spp., including C. trachomatis, C. neumoniae, C. psittaci; Leptsira spp., including L. interrogans; Treponema spp., including T. pallidum, T. denticola, T. hyodysenteriae.

In certain embodiments, first and/or second antigens are derived from a fungal pathogen (e.g., a yeast or mold pathogen). Exemplary fungal pathogens include, e.g., Aspergillus fumigatus, A. flavus, A. niger, A. terreus, A. nidulans, Coccidioides immitis, Coccidioides posadasii, Cryptococcus neoformans, Histoplasma capsulatum, Candida albicans, and Pneumocystis jirovecii.

In certain embodiments, first and/or second antigens are derived from a protozoan pathogen. Exemplary protozoan pathogens include, e.g., Toxoplasma gondii, Strongyloides stercoralis, Plasmodium falciparum, Plasmodium vivax, Plasmodium ovale and Plasmodium malariae.

In certain embodiments, the first and/or second antigens are derived from a multi-cellular parasitic pathogen. Exemplary multicellular parasitic pathogens include, e.g., trematodes (flukes), cestodes (tapeworms), nematodes (roundworms), and arthropods.

In some embodiments, the first and/or second antigens are derived from an allergen, such as pollen allergens (tree-, herb, weed-, and grass pollen allergens); insect or arachnid allergens (inhalant, saliva and venom allergens, e.g. mite allergens, cockroach and midges allergens, hymenopthera venom allergens); animal hair and dandruff allergens (from e.g. dog, cat, horse, rat, mouse, etc.); and food allergens (e.g. a gliadin). Important pollen allergens from trees, grasses and herbs are such originating from the taxonomic orders of Fagales, Oleales, Pinales and platanaceae including, but not limited to, birch (Betula), alder (Alnus), hazel (Corylus), hornbeam (Carpinus) and olive (Olea), cedar (Cryptomeria and Juniperus), plane tree (Platanus), the order of Poales including grasses of the genera Lolium, Phleum, Poa, Cynodon, Dactylis, Holcus, Phalaris, Secale, and Sorghum, the orders of Asterales and Urticales including herbs of the genera Ambrosia, Artemisia, and Parietaria. Other important inhalation allergens are those from house dust mites of the genus Dermatophagoides and Euroglyphus, storage mite e.g. Lepidoglyphys, Glycyphagus and Tyrophagus, those from cockroaches, midges and fleas e.g. Blatella, Periplaneta, Chironomus and Ctenocepphalides, and those from mammals such as cat, dog and horse, venom allergens including such originating from stinging or biting insects such as those from the taxonomic order of Hymenoptera including bees (Apidae), wasps (Vespidea), and ants (Formicoidae).

In some embodiments, the first and/or second antigens are derived from a tumor antigen selected from: (a) cancer-testis antigens such as NY-ESO-1, SSX2, SCP1 as well as RAGE, BAGE, GAGE and MAGE family polypeptides, for example, GAGE-1, GAGE-2, MAGE-1, MAGE-2, MAGE-3, MAGE-4, MAGE-5, MAGE-6, and MAGE-12 (which can be used, for example, to address melanoma, lung, head and neck, NSCLC, breast, gastrointestinal, and bladder tumors; (b) mutated antigens, for example, p53 (associated with various solid tumors, e.g., colorectal, lung, head and neck cancer), p21/Ras (associated with, e.g., melanoma, pancreatic cancer and colorectal cancer), CDK4 (associated with, e.g., melanoma), MUM1 (associated with, e.g., melanoma), caspase-8 (associated with, e.g., head and neck cancer), CIA 0205 (associated with, e.g., bladder cancer), HLA-A2-R1701, beta catenin (associated with, e.g., melanoma), TCR (associated with, e.g., T-cell non-Hodgkins lymphoma), BCR-abl (associated with, e.g., chronic myelogenous leukemia), triosephosphate isomerase, MA 0205, CDC-27, and LDLR-FUT; (c) over-expressed antigens, for example, Galectin 4 (associated with, e.g., colorectal cancer), Galectin 9 (associated with, e.g., Hodgkin's disease), proteinase 3 (associated with, e.g., chronic myelogenous leukemia), WT 1 (associated with, e.g., various leukemias), carbonic anhydrase (associated with, e.g., renal cancer), aldolase A (associated with, e.g., lung cancer), PRAME (associated with, e.g., melanoma), HER-2/neu (associated with, e.g., breast, colon, lung and ovarian cancer), mammaglobin, alpha-fetoprotein (associated with, e.g., hepatoma), KSA (associated with, e.g., colorectal cancer), gastrin (associated with, e.g., pancreatic and gastric cancer), telomerase catalytic protein, MUC-1 (associated with, e.g., breast and ovarian cancer), G-250 (associated with, e.g., renal cell carcinoma), p53 (associated with, e.g., breast, colon cancer), and carcinoembryonic antigen (associated with, e.g., breast cancer, lung cancer, and cancers of the gastrointestinal tract such as colorectal cancer); (d) shared antigens, for example, melanoma-melanocyte differentiation antigens such as MART-1/Melan A, gp100, MC1R, melanocyte-stimulating hormone receptor, tyrosinase, tyrosinase related protein-1/TRP1 and tyrosinase related protein-2/TRP2 (associated with, e.g., melanoma); (e) prostate associated antigens such as PAP, PSA, PSMA, PSH-P1, PSM-P1, PSM-P2, associated with e.g., prostate cancer; (f) immunoglobulin idiotypes (associated with myeloma and B cell lymphomas, for example). In certain embodiments, tumor immunogens include, but are not limited to, p15, Hom/Me1-40, H-Ras, E2A-PRL, H4-RET, IGH-IGK, MYL-RAR, Epstein Barr virus antigens, EBNA, human papillomavirus (HPV) antigens, including E6 and E7, hepatitis B and C virus antigens, human T-cell lymphotropic virus antigens, TSP-180, p185erbB2, p180erbB-3, c-met, mn-23H1, TAG-72-4, CA 19-9, CA 72-4, CAM 17.1, NuMa, K-ras, p16, TAGE, PSCA, CT7, 43-9F, 5T4, 791 Tgp72, beta-HCG, BCA225, BTAA, CA 125, CA 15-3 (CA 27.29\BCAA), CA 195, CA 242, CA-50, CAM43, CD68\KP1, CO-029, FGF-5, Ga733 (EpCAM), HTgp-175, M344, MA-50, MG7-Ag, MOV18, NB/70K, NY-CO-1, RCAS1, SDCCAG16, TA-90 (Mac-2 binding protein/cyclophilin C-associated protein), TAAL6, TAG72, TLP, TPS, and the like.

1. CMV

In certain embodiments, the first or second antigen is from CMV. In certain embodiments, the first or second antigen is derived from a capsid protein, an envelope glycoprotein (such as gB, gH, gL, gM, gN), or a tegument protein. In certain embodiments, the first or second antigen is derived from one or more of the following proteins: pp65, IE1, gB, gD, gH, gL, gM, gN, gO, UL128, UL129, gUL130, UL150, UL131, UL33, UL78, US27, US28, RL5A, RL6, RL10, RL11, RL12, RL13, UL1, UL2, UL4, UL5, UL6, UL7, ULB, UL9, UL10, UL11, UL14, UL15A, UL16, UL17, UL18, UL22A, UL38, UL40, UL41A, UL42, UL116, UL119, UL120, UL121, UL124, UL132, UL147A, UL148, UL142, UL144, UL141, UL140, UL135, UL136, UL138, UL139, UL133, UL135, UL148A, UL148B, UL148C, UL148D, US2, US3, US6, US7, USB, US9, US10, US11, US12, US13, US14, US15, US16, US17, US18, US19, US20, US21, US29, US30, or US34A.

The CMV antigen may also be a fusion polypeptide of one or more CMV proteins, such as pp65/IE1 (Reap et al., Vaccine (2007) 25:7441-7449), gH/gL (Chowdary et al., Nature Structural & Molecular Biology, 17, 882-888 (2010)).

Suitable CMV antigens include gB, gH, gL, gO, and can be from any CMV strain. For example, CMV proteins can be from Merlin, AD169, VR1814, Towne, Toledo, TR, PH, TB40, or Fix strains of CMV. Exemplary sequences of CMV proteins that may be used for the invention are shown in Table 1.

TABLE 1

| | |
|---|---|
| Full length gH polynucleotide | (CMV gH FL) SEQ ID NO: 7 |
| Full length gH polypeptide | (CMV gH FL) SEQ ID NO: 8 |
| Full length gL polynucleotide | (CMV gL FL) SEQ ID NO: 11 |
| Full length gL polypeptide | (CMV gL FL) SEQ ID NO: 12 |
| Full length gO polynucleotide | (CMV gO FL) SEQ ID NO: 17 |
| Full length gO polypeptide | (CMV gO FL) SEQ ID NO: 18 |
| gH sol polynucleotide | (CMV gH sol) SEQ ID NO: 9 |
| gH sol polypeptide | (CMV gH sol) SEQ ID NO: 10 |
| Full length UL128 polynucleotide | (CMV UL128 FL) SEQ ID NO: 19 |
| Full length UL128 polypeptide | (CMV UL128 FL) SEQ ID NO: 20 |
| Full length UL130 polynucleotide | (CMV UL130 FL) SEQ ID NO: 21 |
| Full length UL130 polypeptide | (CMV UL130 FL) SEQ ID NO: 22 |
| Full length UL131 polynucleotide | (CMV UL131 FL) SEQ ID NO: 23 |
| Full length UL131 polypeptide | (CMV UL131 FL) SEQ ID NO: 24 |
| Full length gB polynucleotide | (CMV gB FL) SEQ ID NO: 1 |
| Full length gB polypeptide | (CMV gB FL) SEQ ID NO: 2 |
| gB sol 750 polynucleotide | (CMV gB 750) SEQ ID NO: 3 |
| gB sol 750 polypeptide | (CMV gB 750) SEQ ID NO: 4 |
| gB sol 692 polynucleotide | (CMV gB 692) SEQ ID NO: 5 |
| gB sol 692 polypeptide | (CMV gB 692) SEQ ID NO: 6 |
| Full length gM polynucleotide | (CMV gM FL) SEQ ID NO: 13 |
| Full length gM polypeptide | (CMV gM FL) SEQ ID NO: 14 |
| Full length gN polynucleotide | (CMV gN FL) SEQ ID NO: 15 |
| Full length gN polypeptide | (CMV gN FL) SEQ ID NO: 16 |

gB Antigens

In certain embodiments, the first or second antigen may be a gB antigen. A gB antigen can be full length gB protein or can omit one or more regions of the protein. Alternatively, fragments of a gB protein can be used. gB amino acids are numbered according to the full-length gB amino acid sequence (CMV gB FL) shown in SEQ ID NO: 2, which is 907 amino acids long. Suitable regions of a gB protein, which can be excluded from the full-length protein or included as fragments include: the signal sequence (amino acids 1-24), a gB-DLD disintegrin-like domain (amino acids 57-146), a furin cleavage site (amino acids 459-460), a heptad repeat region (679-693), a membrane spanning domain (amino acids 751-771), and a cytoplasmic domain from amino acids 771-906. In some embodiments, a gB antigen includes amino acids 67-86 (Neutralizing Epitope AD2) and/or amino acids 532-635 (Immunodominant Epitope AD1). Specific examples of gB antigens include “gB sol 692,” which includes the first 692 amino acids of gB, and “gB sol 750,” which includes the first 750 amino acids of gB. The signal sequence, amino acids 1-24, can be present or absent from gB sol 692 and gB sol 750 as desired.

In some embodiments, the gB antigen is a gB fragment of 10 amino acids or longer. For example, the number of amino acids in the fragment can comprise 10, 15, 20, 30, 40, 50, 60, 70, 80, 90, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, or 875 amino acids.

The invention may also use a gB antigen comprising an amino acid sequence that is at least 75% identical to SEQ ID NO: 2 (e.g., at least 80%, at least 85%, at least 90%, at least 95%, at least 97%, at least 98%, at least 99%, or 100% identical to SEQ ID NO: 2).

gH Antigens

In certain embodiments, the first or second antigen may be a gH antigen. A gH antigen can be a full-length gH protein (CMV gH FL, SEQ ID NO:8, for example, which is a 743 amino acid protein). gH has a membrane spanning domain and a cytoplasmic domain starting at position 716 to position 743. Removing amino acids from 717 to 743 provides a soluble gH (e.g., CMV gH sol, SEQ ID NO: 10).

In some embodiments, the gH antigen is a gH fragment of 10 amino acids or longer. For example, the number of amino acids in the fragment can comprise 10, 15, 20, 30, 40, 50, 60, 70, 80, 90, 100, 125, 150, 175, 200, 225, 250, 275, 300, 325, 350, 375, 400, 425, 450, 475, 500, 525, 550, 575, 600, 625, 650, 675, 700, or 725 amino acids.

The invention may also use a gH antigen comprising an amino acid sequence that is at least 75% identical to SEQ ID NO: 8 or 10 (e.g., at least 80%, at least 85%, at least 90%, at least 95%, at least 97%, at least 98%, at least 99%, or 100% identical to SEQ ID NO: 8 or 10).

gL Antigens

In certain embodiments, the first or second antigen may be a gL antigen. A gH antigen can be a full-length gL protein (CMV gL FL, SEQ ID NO:12, for example, which is a 278 amino acid protein). Alternatively, a gL fragment can be used. For example, the number of amino acids in the fragment can comprise 10, 15, 20, 30, 40, 50, 60, 70, 80, 90, 100, 125, 150, 175, 200, 225, or 250 amino acids.

The invention may also use a gL antigen comprising an amino acid sequence that is at least 75% identical to SEQ ID NO: 12 (e.g., at least 80%, at least 85%, at least 90%, at least 95%, at least 97%, at least 98%, at least 99%, or 100% identical to SEQ ID NO: 12).

gO Antigens

In certain embodiments, the first or second antigen may be a gO antigen. A gO antigen can be a full-length gO protein (CMV gO FL, SEQ ID NO:18, for example, which is a 472 amino acid protein). Alternatively, the gO antigen can be a gO fragment of 10 amino acids or longer. For example, the number of amino acids in the fragment can comprise 10, 15, 20, 30, 40, 50, 60, 70, 80, 90, 100, 125, 150, 175, 200, 225, 250, 275, 300, 325, 350, 375, 400, 425, or 450 amino acids.

The invention may also use a gO antigen comprising an amino acid sequence that is at least 75% identical to SEQ ID NO: 18 (e.g., at least 80%, at least 85%, at least 90%, at least 95%, at least 97%, at least 98%, at least 99%, or 100% identical to SEQ ID NO: 18).

gM Antigens

In certain embodiments, the first or second antigen may be a gM antigen. A gM antigen can be a full-length gM protein (CMV gM FL, SEQ ID NO:14, for example, which is a 371 amino acid protein). Alternatively, the gM antigen can be a gM fragment of 10 amino acids or longer. For example, the number of amino acids in the fragment can comprise 10, 15, 20, 30, 40, 50, 60, 70, 80, 90, 100, 125, 150, 175, 200, 225, 250, 275, 300, 325, or 350 amino acids.

The invention may also use a gM antigen comprising an amino acid sequence that is at least 75% identical to SEQ ID NO: 14 (e.g., at least 80%, at least 85%, at least 90%, at least 95%, at least 97%, at least 98%, at least 99%, or 100% identical to SEQ ID NO: 14).

gN Antigens

In certain embodiments, the first or second antigen may be a gN antigen. A gN antigen can be a full-length gN protein (CMV gN FL, SEQ ID NO:16, for example, which is a 135 amino acid protein). Alternatively, the gN antigen can be a gN fragment of 10 amino acids or longer. For example, the number of amino acids in the fragment can comprise 10, 15, 20, 30, 40, 50, 60, 70, 80, 90, 100, or 125 amino acids.

The invention may also use a gN antigen comprising an amino acid sequence that is at least 75% identical to SEQ ID NO: 16 (e.g., at least 80%, at least 85%, at least 90%, at least 95%, at least 97%, at least 98%, at least 99%, or 100% identical to SEQ ID NO: 16).

UL128 Antigens

In certain embodiments, the first or second antigen may be a UL128 antigen. A UL128 antigen can be a full-length UL128 protein (CMV UL128 FL, SEQ ID NO:20, for example, which is a 171 amino acid protein). Alternatively, the UL128 antigen can be a UL128 fragment of 10 amino acids or longer. For example, the number of amino acids in the fragment can comprise 10, 15, 20, 30, 40, 50, 60, 70, 80, 90, 100, 125, or 150 amino acids.

The invention may also use a UL128 antigen comprising an amino acid sequence that is at least 75% identical to SEQ ID NO: 20 (e.g., at least 80%, at least 85%, at least 90%, at least 95%, at least 97%, at least 98%, at least 99%, or 100% identical to SEQ ID NO: 20).

UL130 Antigens

In certain embodiments, the first or second antigen may be a UL130 antigen. A UL130 antigen can be a full-length UL130 protein (CMV UL130 FL, SEQ ID NO:22, for example, which is a 214 amino acid protein). Alternatively, the UL130 antigen can be a UL130 fragment of 10 amino acids or longer. For example, the number of amino acids in the fragment can comprise 10, 15, 20, 30, 40, 50, 60, 70, 80, 90, 100, 125, 150, 175, or 200 amino acids.

The invention may also use a UL130 antigen comprising an amino acid sequence that is at least 75% identical to SEQ ID NO: 22 (e.g., at least 80%, at least 85%, at least 90%, at least 95%, at least 97%, at least 98%, at least 99%, or 100% identical to SEQ ID NO: 22).

UL131 Antigens

In certain embodiments, the first or second antigen may be a UL131 antigen. A UL131 antigen can be a full-length UL131 protein (CMV UL131, SEQ ID NO:24, for example, which is a 129 amino acid protein). Alternatively, the UL131 antigen can be a UL131 fragment of 10 amino acids or longer. For example, the number of amino acids in the fragment can comprise 10, 15, 20, 30, 40, 50, 60, 70, 80, 90, 100, 125, 150, 175, or 200 amino acids.

The invention may also use a UL131 antigen comprising an amino acid sequence that is at least 75% identical to SEQ ID NO: 24 (e.g., at least 80%, at least 85%, at least 90%, at least 95%, at least 97%, at least 98%, at least 99%, or 100% identical to SEQ ID NO: 24).

The CMV antigen may be a fusion polypeptide. For example, the antigen may comprise a first domain and a second domain, wherein (i) the first domain comprises a first CMV antigen and (ii) the second domain comprises a second CMV antigen. The first CMV antigen and the second CMV antigen are independently selected from a gB, gH, gL, gO, gM, gN, UL128, UL130, or UL131 antigen described above.

Complexes

Two or more CMV antigens may also be co-delivered so that they form a complex in vivo (e.g., gH/gL complex, gM/gN complex, gH/gL/UL128/UL130/UL131 pentameric complex). For example, the immunogenic composition may comprise an RNA molecule that encode two separate antigens, gH and gL. The immunogenic composition may also comprise two polypeptide antigens, gH and gL.

2. Parvovirus

In certain embodiments, the polypeptide antigen is derived from Parvovirus. Preferrably, the parvovirus infects humans, i.e., is of the Dependovirus, Erythrovirus, or Bocavirus genus. In certain embodiments, the parvovirus is parvovirus B19. In some embodiments, the parvovirus is from the Parvovirus genus. Parvovirus B19 belongs to the Erythroviruses genus of the Parvoviridae family of small DNA viruses. It is a non-enveloped, icosahedral virus that contains a single-stranded linear DNA genome. The parvovirus B19 virion is 20-25 nm in diameter and has a genome of 5.6 kb (Clewley, 1984, Cotmore & Tattersall, 1984). The parvovirus B19 capsid consists of an 83 kDa minor structural protein, VP1, and a 58 kDa major structural protein, VP2. It has a non-segmented single stranded DNA genome surrounded by a protein shell containing two structural proteins, VP1 and VP2 in a ˜5% to ˜95% ratio (Ozawa et al., 1987). The sequences of the two proteins are co-linear, with VP2 being identical to the carboxyl-terminal region of VP1; however, VP1 comprises an additional 227 amino acids at the amino-terminus. Long lasting antibody responses are directed to both VP1 and VP2 proteins and thus these proteins alone are expected to raise a significant immune response.

The parvovirus B19 genome contains three open reading frames: a non-structural 77 kDa protein, NS1, is encoded by nucleotides 436-2451; the minor structural protein, VP1 is encoded by nucleotides 2444-4787, and the major structural protein, VP2, is encoded by nucleotides 3125-4787 (Corcoran et al., J. Med. Microb., 2004). Parvovirus B19 uses a single promoter, p6, which is capable of expressing structural and non-structural genes differentially (Blundell et al., 1987, Ozawa et al., 1987). Although the foregoing numbering is relative to the nucleotide sequence of the parvovirus B19 genome, it is to be understood that the corresponding positions in sequences obtained from other genotypes and isolates of parvovirus are also intended to be encompassed by the present invention. Any one of the VP1 or VP2 proteins, as well as variants thereof, such as immunogenic fragments thereof, and nucleic acids encoding such proteins can be used in the practice of the invention.

In certain embodiments, the Parvovirus antigen is a VLP. The VLP can contain VP1 and VP2 from any desired parvovirus, or any desired combination. In some embodiments the VP1 and VP2 proteins can have an amino acid sequence that is the same as or substantially the same as a naturally occurring parvovirus VP1 or VP2, or can contain one or more amino acid substitutions, deletions or additions. For example, VP1 can be mutated to inactivate its phospholipase activity. For example, the amino acid sequence of VP1 may contain a point mutation (e.g., His153A1a), or any of the mutations described in WO 06/032697, EP 1791858 or US 20070286870. Preferably, the VLP contains VP1 and VP2 are from a parvovirus that infects humans, i.e., a parvovirus of the Dependovirus, Erythrovirus, or Bocavirus genus. In certain embodiments, the VLP contains parvovirus B19 VP1 and parvovirus B19 VP2.

In certain embodiments, the VLP comprises VP1 in lower abundance relative to VP2 (e.g., soluble VP1 is produced in lower abundance than soluble VP2), as a result of the individual control elements that are operably linked to the nucleic acids that encode VP1 and VP2 and/or as a result of other features of the recombinant nucleic acids that encode VP1 and VP2, such as optimized codon usage and deoptimized codon usage. Such control elements (e.g., promoters) and features (e.g., codon usage) allow for the relative production of VP1 and VP2 to be controlled.

Exemplary sequences of Parvovirus proteins that may be used for the invention are shown in Table 2.

TABLE 2
Parvovirus antigens

| | |
|---|---|
| Parvovirus | (ParvoB19.Opti. VP1) polynucleotide SEQ ID |
| B19 VP1 | NO: 25 (polypeptide encoded by the open |
| | reading fram shown in upper case in SEQ ID |
| | NO: 25) |
| Parvovirus | (ParvoB19.Opti. VP2) polynucleotide SEQ ID |
| B19 VP2 | NO: 26 (polypeptide encoded by the open |
| | reading fram shown in upper case in SEQ ID |
| | NO: 26) |

3. RSV

In some aspects, the pathogen is RSV. RSV is an enveloped non-segmented negative-strand RNA virus in the family Paramyxoviridae, genus Pneumovirus. To infect a host cell, paramyxoviruses such as RSV, like other enveloped viruses such as influenza virus and HIV, require fusion of the viral membrane with a host cell's membrane. For RSV, the conserved fusion protein (RSV-F glycoprotein) fuses the viral and cellular membranes by coupling irreversible protein refolding with juxtaposition of the membranes. In current models based on paramyxovirus studies, the RSV-F protein initially folds into a metastable “pre-fusion” conformation. During cell entry, the pre-fusion conformation undergoes refolding and conformational changes to its stable “post-fusion” conformation. See, also, Swanson et al., PNAS USA 108(23):9619-9624 (2011) regarding pre-fusion and post-fusion RSV-F structures.

In certain embodiments, the first and second antigens are from RSV. For example, the first and second antigens can independently be derived from the RSV surface glycoproteins Fusion (F), Glycoprotein (G), Small Hydrophobic protein (SH), the matrix proteins M and M2, the nucleocapsid proteins N, P and L, and the nonstructural proteins NS1 and NS2. In certain preferred embodiments, the first and second antigens are each an RSV-F antigen.

The F glycoprotein of RSV is a type I single-pass integral membrane protein having four general domains: N-terminal ER-translocating signal sequence (SS), ectodomain (ED), transmembrane domain (TM), and a cytoplasmic tail (CT). CT contains a single palmitoylated cysteine residue. The sequence of F protein is highly conserved among RSV isolates, but is constantly evolving (Kim et al. (2007) J Med Virol 79: 820-828). Unlike most paramyxoviruses, the F protein in RSV can mediate entry and syncytium formation independent of the other viral proteins (HN is usually necessary in addition to F in other paramyxoviruses).

The RSV-F glycoprotein is translated from mRNA into an approximately 574 amino acid protein designated F₀. Post-translational processing of F₀ includes removal of an N-terminal signal peptide by a signal peptidase in the endoplasmic reticulum. F₀ is also cleaved at two sites (approximately 109/110 and approximately 136/137) by cellular proteases (in particular furin) in the trans-Golgi. This cleavage results in the removal of a short intervening sequence and generates two subunits designated F₁ (−50 kDa; C-terminal; approximately residues 137-574) and F₂ (—20 kDa; N-terminal; approximately residues 1-109) that remain associated with each other. F₁ contains a hydrophobic fusion peptide at its N-terminus and also two amphipathic heptad-repeat regions (HRA and HRB). HRA is near the fusion peptide and HRB is near the transmembrane domain. Three F₁-F₂ heterodimers are assembled as homotrimers of F₁-F₂ in the virion.

RSV-F antigens suitable for inclusion in the immunogenic compositions described herein, either in RNA encoded form or as polypeptides, include RSV-F glycoprotein and RSV-F glycoprotein variants. Suitable RSV-F glycoprotein variants include, for example, full length F protein and truncated variants such as soluble ecto-domains, each optionally containing one or more mutations, such as furin-cleavage mutations, trypsin-cleavage mutations, fusion peptide mutations (e.g., deletions in whole or in part), mutations that stabilize the HRB trimer, and mutations that destabilize the HRA trimer.

Full length and truncated RSV-F glycoprotiens, including those with one or more such mutations in a variety of combinations are well known in the art and are disclosed for example in WO2011/008974, the disclosure of which is incorporated herein by reference in its entirety.

The skilled addressee is directed to the following sections of WO2011/008974 which disclose exemplary RSV-F antigens that can be used, in RNA form or as polypeptides, in the immunogenic compositions: (i) page 15, line 20 through page 16, line 27, which describes RSV-F, its amino acid sequence and domain structure; (ii) page 16, line 28 through page 18, line 11, which describes soluble ectodomains of RSV-F; (iii) page 18, line 14 through page 20, line 15, which describes furin-cleavage mutations, trypsin-cleavage mutations, fusion peptide mutations; (iv) page 20, line 16 through page 21, line 8, and page 26, line 29 through page 30, line 14, which describe optional oligomerization sequences; (v) page 20, lines 9-24, which describe introduced protease cleavage sites; (vi) and page 30, line 18 through page 32, line 18, which describe mutations that stabilize the HRB trimer, destabilize the HRA trimer and other mutations that can be included.

B. The RNA Molecule

The immunogenic composition described herein comprises an RNA component and a polypeptide component. Preferably, the RNA is a self-replicating RNA.

The composition can contain more than one RNA molecule encoding an antigen, e.g., two, three, five, ten or more RNA molecules. Alternatively or in addition, one RNA molecule may also encode more than one antigen, e.g., a bicistronic, or tricistronic RNA molecule that encodes different or identical antigens.

The sequence of the RNA molecule may be codon optimized or deoptimized for expression in a desired host, such as a human cell.

The sequence of the RNA molecule may be modified if desired, for example to increase the efficacy of expression or replication of the RNA, or to provide additional stability or resistance to degradation. For example, the RNA sequence can be modified with respect to its codon usage, for example, to increase translation efficacy and half-life of the RNA. A poly A tail (e.g., of about 30 adenosine residues or more) may be attached to the 3′ end of the RNA to increase its half-life. The 5′ end of the RNA may be capped with a modified ribonucleotide with the structure m7G (5′) ppp (5′) N (cap 0 structure) or a derivative thereof, which can be incorporated during RNA synthesis or can be enzymatically engineered after RNA transcription (e.g., by using Vaccinia Virus Capping Enzyme (VCE) consisting of mRNA triphosphatase, guanylyl-transferase and guanine-7-methytransferase, which catalyzes the construction of N7-monomethylated cap 0 structures). Cap 0 structure plays an important role in maintaining the stability and translational efficacy of the RNA molecule. The 5′ cap of the RNA molecule may be further modified by a 2′-O-Methyltransferase which results in the generation of a cap 1 structure (m7Gppp [m2′-O]N), which may further increases translation efficacy.

If desired, the RNA molecule can comprise one or more modified nucleotides in addition to any 5′ cap structure. There are more than 96 naturally occurring nucleoside modifications found on mammalian RNA. See, e.g., Limbach et al., Nucleic Acids Research, 22(12):2183-2196 (1994). The preparation of nucleotides and modified nucleotides and nucleosides are well-known in the art, e.g. from U.S. Pat. Nos. 4,373,071, 4,458,066, 4,500,707, 4,668,777, 4,973,679, 5,047,524, 5,132,418, 5,153,319, 5,262,530, 5,700,642 all of which are incorporated by reference in their entirety herein, and many modified nucleosides and modified nucleotides are commercially available.

Modified nucleobases which can be incorporated into modified nucleosides and nucleotides and be present in the RNA molecules include: m5C (5-methylcytidine), m5U (5-methyluridine), m6A (N6-methyladenosine), s2U (2-thiouridine), Um (2′-O-methyluridine), ml A (1-methyladenosine); m2A (2-methyladenosine); Am (2-1-O-methyladenosine); ms2m6A (2-methylthio-N6-methyladenosine); i6A (N6-isopentenyladenosine); ms2i6A (2-methylthio-N6isopentenyladenosine); io6A (N6-(cis-hydroxyisopentenyl)adenosine); ms2io6A (2-methylthio-N6-(cis-hydroxyisopentenyl) adenosine); g6A (N6-glycinylcarbamoyladenosine); t6A (N6-threonyl carbamoyladenosine); ms2t6A (2-methylthio-N6-threonyl carbamoyladenosine); m6t6A (N6-methyl-N6-threonylcarbamoyladenosine); hn6A(N6-hydroxynorvalylcarbamoyl adenosine); ms2hn6A (2-methylthio-N6-hydroxynorvalyl carbamoyladenosine); Ar(p) (2′-O-ribosyladenosine (phosphate)); I (inosine); m1I (1-methylinosine); m′Im (1,2′-O-dimethylinosine); m3C (3-methylcytidine); Cm (2T-O-methylcytidine); s2C (2-thiocytidine); ac4C (N4-acetylcytidine); f5C (5-fonnylcytidine); m5Cm (5,2-O-dimethyl cytidine); ac4Cm (N4acetyl2TOmethylcytidine); k2C (lysidine); m1G (1-methylguanosine); m2G (N2-methylguanosine); m7G (7-methylguanosine); Gm (2′-O-methylguanosine); m22G (N2,N2-dimethylguanosine); m2Gm (N2,2′-O-dimethylguanosine); m22Gm (N2,N2,2′-O-trimethylguanosine); Gr(p) (2′-O-ribosylguanosine (phosphate)); yW (wybutosine); o2yW (peroxywybutosine); OHyW (hydroxywybutosine); OHyW* (undermodified hydroxywybutosine); imG (wyosine); mimG (methylguanosine); Q (queuosine); oQ (epoxyqueuosine); galQ (galtactosyl-queuosine); manQ (mannosyl-queuosine); preQo (7-cyano-7-deazaguanosine); preQi (7-aminomethyl-7-deazaguanosine); G* (archaeosine); D (dihydrouridine); m5Um (5,2′-O-dimethyluridine); s4U (4-thiouridine); m5s2U (5-methyl-2-thiouridine); s2Um (2-thio-2′-O-methyluridine); acp3U (3-(3-amino-3-carboxypropyl)uridine); ho5U (5-hydroxyuridine); mo5U (5-methoxyuridine); cmo5U (uridine 5-oxyacetic acid); mcmo5U (uridine 5-oxyacetic acid methyl ester); chm5U (5-(carboxyhydroxymethyl)uridine)); mchm5U (5-(carboxyhydroxymethyl)uridine methyl ester); mcm5U (5-methoxycarbonyl methyluridine); mcm5Um (S-methoxycarbonylmethyl-2-O-methyluridine); mcm5s2U (5-methoxycarbonylmethyl-2-thiouridine); nm5s2U (5-aminomethyl-2-thiouridine); mnm5U (5-methylaminomethyluridine); nmm5s2U (5-methylaminomethyl-2-thiouridine); nmm5se2U (5-methylaminomethyl-2-selenouridine); ncm5U (5-carbamoylmethyl uridine); ncm5Um (5-carbamoylmethyl-2′-O-methyluridine); cmnm5U (5-carboxymethylaminomethyluridine); cnmm5Um (5-carboxymethylaminomethyl-2-L-Omethyluridine); cmnm5s2U (5-carboxymethylaminomethyl-2-thiouridine); m62A (N6,N6-dimethyladenosine); Tm (2′-O-methylinosine); m4C (N4-methylcytidine); m4Cm (N4,2-O-dimethylcytidine); hm5C (5-hydroxymethylcytidine); m3U (3-methyluridine); cm5U (5-carboxymethyluridine); m6Am (N6,T-O-dimethyladenosine); m62Am (N6,N6, O-2-trimethyladenosine); m2′7G (N2,7-dimethylguanosine); m2′2′7G (N2,N2,7-trimethylguanosine); m3Um (3,2T-O-dimethyluridine); m5D (5-methyldihydrouridine); f5Cm (5-formyl-2′-O-methylcytidine); m1Gm (1,2′-O-dimethylguanosine); m′Am (1,2-O-dimethyl adenosine) irinomethyluridine); tm5s2U (S-taurinomethyl-2-thiouridine)); imG-14 (4-demethyl guanosine); imG2 (isoguanosine); ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(C1-C₆)-alkyluracil, 5-methyluracil, 5-(C2-C₆)-alkenyluracil, 5-(C2-C₆)-alkynyluracil, 5-(hydroxymethyl)uracil, 5-chlorouracil, 5-fluorouracil, 5-bromouracil, 5-hydroxycytosine, 5-(C1-C₆)-alkylcytosine, 5-methylcytosine, 5-(C2-C₆)-alkenylcytosine, 5-(C2-C₆)-alkynylcytosine, 5-chlorocytosine, 5-fluorocytosine, 5-bromocytosine, N²-dimethylguanine, 7-deazaguanine, 8-azaguanine, 7-deaza-7-substituted guanine, 7-deaza-7-(C2-C₆)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, hydrogen (abasic residue), mSC, mSU, m6A, s2U, W, or 2′-O-methyl-U. Many of these modified nucleobases and their corresponding ribonucleosides are available from commercial suppliers. See, e.g., WO 2011/005799 which is incorporated herein by reference.

If desired, the RNA molecule can contain phosphoramidate, phosphorothioate, and/or methylphosphonate linkages.

In some embodiments, the RNA molecule does not include modified nucleotides, e.g., does not include modified nucleobases, and all of the nucleotides in the RNA molecule are conventional standard ribonucleotides A, U, G and C, with the exception of an optional 5′ cap that may include, for example, 7-methylguanosine. In other embodiments, the RNA may include a 5′ cap comprising a 7′-methylguanosine, and the first 1, 2 or 3 5′ ribonucleotides may be methylated at the 2′ position of the ribose.

Self-Replicating RNA

In some aspects, the cationic oil in water emulsion contains a self-replicating RNA molecule. In certain embodiments, the self-replicating RNA molecule is derived from or based on an alphavirus.

Self-replicating RNA molecules are well known in the art and can be produced by using replication elements derived from, e.g., alphaviruses, and substituting the structural viral proteins with a nucleotide sequence encoding a protein of interest. Cells transfected with self-replicating RNA briefly produce of antigen before undergoing apoptotic death. This death is a likely result of requisite double-stranded (ds) RNA intermediates, which also have been shown to super-activate Dendritic Cells. Thus, the enhanced immunogenicity of self-replicating RNA may be a result of the production of pro-inflammatory dsRNA, which mimics an RNA-virus infection of host cells.

Advantageously, the cell's machinery is used by self-replicating RNA molecules to generate an exponential increase of encoded gene products, such as proteins or antigens, which can accumulate in the cells or be secreted from the cells. Overexpression of proteins or antigens by self-replicating RNA molecules takes advantage of the immunostimulatory adjuvant effects, including stimulation of toll-like receptors (TLR) 3, 7 and 8 and non TLR pathways (e.g, RIG-1, MD-5) by the products of RNA replication and amplification, and translation which induces apoptosis of the transfected cell.

The self-replicating RNA generally contains at least one or more genes selected from the group consisting of viral replicases, viral proteases, viral helicases and other nonstructural viral proteins, and also comprise 5′- and 3′-end cis-active replication sequences, and if desired, a heterologous sequences that encode a desired amino acid sequences (e.g., an antigen of interest). A subgenomic promoter that directs expression of the heterologous sequence can be included in the self-replicating RNA. If desired, the heterologous sequence (e.g., an antigen of interest) may be fused in frame to other coding regions in the self-replicating RNA and/or may be under the control of an internal ribosome entry site (IRES).

In certain embodiments, the self-replicating RNA molecule is not encapsulated in a virus-like particle. Self-replicating RNA molecules of the invention can be designed so that the self-replicating RNA molecule cannot induce production of infectious viral particles. This can be achieved, for example, by omitting one or more viral genes encoding structural proteins that are necessary for the production of viral particles in the self-replicating RNA. For example, when the self-replicating RNA molecule is based on an alpha virus, such as Sinebis virus (SIN), Semliki forest virus and Venezuelan equine encephalitis virus (VEE), one or more genes encoding viral structural proteins, such as capsid and/or envelope glycoproteins, can be omitted.

If desired, self-replicating RNA molecules of the invention can also be designed to induce production of infectious viral particles that are attenuated or virulent, or to produce viral particles that are capable of a single round of subsequent infection.

When delivered to a vertebrate cell, a self-replicating RNA molecule can lead to the production of multiple daughter RNAs by transcription from itself (or from an antisense copy of itself). The self-replicating RNA can be directly translated after delivery to a cell, and this translation provides a RNA-dependent RNA polymerase which then produces transcripts from the delivered RNA. Thus the delivered RNA leads to the production of multiple daughter RNAs. These transcripts are antisense relative to the delivered RNA and may be translated themselves to provide in situ expression of a gene product, or may be transcribed to provide further transcripts with the same sense as the delivered RNA which are translated to provide in situ expression of the gene product.

One suitable system for achieving self-replication is to use an alphavirus-based RNA replicon. Alphaviruses comprise a set of genetically, structurally, and serologically related arthropod-borne viruses of the Togaviridae family. Twenty-six known viruses and virus subtypes have been classified within the alphavirus genus, including, Sindbis virus, Semliki Forest virus, Ross River virus, and Venezuelan equine encephalitis virus. As such, the self-replicating RNA of the invention may incorporate a RNA replicase derived from semliki forest virus (SFV), sindbis virus (SIN), Venezuelan equine encephalitis virus (VEE), Ross-River virus (RRV), or other viruses belonging to the alphavirus family.

An alphavirus-based “replicon” expression vectors can be used in the invention. Replicon vectors may be utilized in several formats, including DNA, RNA, and recombinant replicon particles. Such replicon vectors have been derived from alphaviruses that include, for example, Sindbis virus (Xiong et al. (1989) Science 243:1188-1191; Dubensky et al., (1996) J. Virol. 70:508-519; Hariharan et al. (1998) J. Virol. 72:950-958; Polo et al. (1999) PNAS 96:4598-4603), Semliki Forest virus (Liljestrom (1991) Bio/Technology 9:1356-1361; Berglund et al. (1998) Nat. Biotech. 16:562-565), and Venezuelan equine encephalitis virus (Pushko et al. (1997) Virology 239:389-401). Alphaviruses-derived replicons are generally quite similar in overall characteristics (e.g., structure, replication), individual alphaviruses may exhibit some particular property (e.g., receptor binding, interferon sensitivity, and disease profile) that is unique. Therefore, chimeric alphavirus replicons made from divergent virus families may also be useful.

Alphavirus-based replicons are (+)-stranded replicons that can be translated after delivery to a cell to give of a replicase (or replicase-transcriptase). The replicase is translated as a polyprotein which auto-cleaves to provide a replication complex which creates genomic (−)-strand copies of the +-strand delivered RNA. These (−)-strand transcripts can themselves be transcribed to give further copies of the (+)-stranded parent RNA and also to give a subgenomic transcript which encodes the desired gene product. Translation of the subgenomic transcript thus leads to in situ expression of the desired gene product by the infected cell. Suitable alphavirus replicons can use a replicase from a sindbis virus, a semliki forest virus, an eastern equine encephalitis virus, a venezuelan equine encephalitis virus, etc.

A preferred self-replicating RNA molecule thus encodes (i) a RNA-dependent RNA polymerase which can transcribe RNA from the self-replicating RNA molecule and (ii) a polypeptide antigen. The polymerase can be an alphavirus replicase e.g. comprising alphavirus protein nsP4.

Whereas natural alphavirus genomes encode structural virion proteins in addition to the non-structural replicase, it is preferred that an alphavirus based self-replicating RNA molecule of the invention does not encode alphavirus structural proteins. Thus the self-replicating RNA can lead to the production of genomic RNA copies of itself in a cell, but not to the production of RNA-containing alphavirus virions. The inability to produce these virions means that, unlike a wild-type alphavirus, the self-replicating RNA molecule cannot perpetuate itself in infectious form. The alphavirus structural proteins which are necessary for perpetuation in wild-type viruses are absent from self-replicating RNAs of the invention and their place is taken by gene(s) encoding the desired gene product, such that the subgenomic transcript encodes the desired gene product rather than the structural alphavirus virion proteins.

Thus a self-replicating RNA molecule useful with the invention may have two open reading frames. The first (5′) open reading frame encodes a replicase; the second (3′) open reading frame encodes a polypeptide antigen. In some embodiments the RNA may have additional (downstream) open reading frames e.g. that encode another desired gene products. A self-replicating RNA molecule can have a 5′ sequence which is compatible with the encoded replicase.

In other aspects, the self-replicating RNA molecule is derived from or based on a virus other than an alphavirus, preferably, a positive-stranded RNA virus, and more preferably a picornavirus, flavivirus, rubivirus, pestivirus, hepacivirus, calicivirus, or coronavirus. Suitable wild-type alphavirus sequences are well-known and are available from sequence depositories, such as the American Type Culture Collection, Rockville, Md. Representative examples of suitable alphaviruses include Aura (ATCC VR-368), Bebaru virus (ATCC VR-600, ATCC VR-1240), Cabassou (ATCC VR-922), Chikungunya virus (ATCC VR-64, ATCC VR-1241), Eastern equine encephalomyelitis virus (ATCC VR-65, ATCC VR-1242), Fort Morgan (ATCC VR-924), Getah virus (ATCC VR-369, ATCC VR-1243), Kyzylagach (ATCC VR-927), Mayaro (ATCC VR-66), Mayaro virus (ATCC VR-1277), Middleburg (ATCC VR-370), Mucambo virus (ATCC VR-580, ATCC VR-1244), Ndumu (ATCC VR-371), Pixuna virus (ATCC VR-372, ATCC VR-1245), Ross River virus (ATCC VR-373, ATCC VR-1246), Semliki Forest (ATCC VR-67, ATCC VR-1247), Sindbis virus (ATCC VR-68, ATCC VR-1248), Tonate (ATCC VR-925), Triniti (ATCC VR-469), Una (ATCC VR-374), Venezuelan equine encephalomyelitis (ATCC VR-69, ATCC VR-923, ATCC VR-1250 ATCC VR-1249, ATCC VR-532), Western equine encephalomyelitis (ATCC VR-70, ATCC VR-1251, ATCC VR-622, ATCC VR-1252), Whataroa (ATCC VR-926), and Y-62-33 (ATCC VR-375).

The self-replicating RNA molecules of the invention are larger than other types of RNA (e.g. mRNA). Typically, the self-replicating RNA molecules of the invention contain at least about 4 kb. For example, the self-replicating RNA can contain at least about 5 kb, at least about 6 kb, at least about 7 kb, at least about 8 kb, at least about 9 kb, at least about 10 kb, at least about 11 kb, at least about 12 kb or more than 12 kb. In certain examples, the self-replicating RNA is about 4 kb to about 12 kb, about 5 kb to about 12 kb, about 6 kb to about 12 kb, about 7 kb to about 12 kb, about 8 kb to about 12 kb, about 9 kb to about 12 kb, about 10 kb to about 12 kb, about 11 kb to about 12 kb, about 5 kb to about 11 kb, about 5 kb to about 10 kb, about 5 kb to about 9 kb, about 5 kb to about 8 kb, about 5 kb to about 7 kb, about 5 kb to about 6 kb, about 6 kb to about 12 kb, about 6 kb to about 11 kb, about 6 kb to about 10 kb, about 6 kb to about 9 kb, about 6 kb to about 8 kb, about 6 kb to about 7 kb, about 7 kb to about 11 kb, about 7 kb to about 10 kb, about 7 kb to about 9 kb, about 7 kb to about 8 kb, about 8 kb to about 11 kb, about 8 kb to about 10 kb, about 8 kb to about 9 kb, about 9 kb to about 11 kb, about 9 kb to about 10 kb, or about 10 kb to about 11 kb.

The self-replicating RNA molecules of the invention may comprise one or more modified nucleotides (e.g., pseudouridine, N6-methyladenosine, 5-methylcytidine, 5-methyluridine).

The self-replicating RNA molecule may encode a single polypeptide antigen or, optionally, two or more of polypeptide antigens linked together in a way that each of the sequences retains its identity (e.g., linked in series) when expressed as an amino acid sequence. The polypeptides generated from the self-replicating RNA may then be produced as a fusion polypeptide or engineered in such a manner to result in separate polypeptide or peptide sequences.

The self-replicating RNA of the invention may encode one or more polypeptide antigens that contain a range of epitopes. Preferably epitopes capable of eliciting either a helper T-cell response or a cytotoxic T-cell response or both.

The self-replicating RNA molecules described herein may be engineered to express multiple nucleotide sequences, from two or more open reading frames, thereby allowing co-expression of proteins, such as a two or more antigens together with cytokines or other immunomodulators, which can enhance the generation of an immune response. Such a self-replicating RNA molecule might be particularly useful, for example, in the production of various gene products (e.g., proteins) at the same time, for example, as a bivalent or multivalent vaccine.

The self-replicating RNA molecules of the invention can be prepared using any suitable method. Several suitable methods are known in the art for producing RNA molecules that contain modified nucleotides. For example, a self-replicating RNA molecule that contains modified nucleotides can be prepared by transcribing (e.g., in vitro transcription) a DNA that encodes the self-replicating RNA molecule using a suitable DNA-dependent RNA polymerase, such as T7 phage RNA polymerase, SP6 phage RNA polymerase, T3 phage RNA polymerase, and the like, or mutants of these polymerases which allow efficient incorporation of modified nucleotides into RNA molecules. The transcription reaction will contain nucleotides and modified nucleotides, and other components that support the activity of the selected polymerase, such as a suitable buffer, and suitable salts. The incorporation of nucleotide analogs into a self-replicating RNA may be engineered, for example, to alter the stability of such RNA molecules, to increase resistance against RNases, to establish replication after introduction into appropriate host cells (“infectivity” of the RNA), and/or to induce or reduce innate and adaptive immune responses.

Suitable synthetic methods can be used alone, or in combination with one or more other methods (e.g., recombinant DNA or RNA technology), to produce a self-replicating RNA molecule of the invention. Suitable methods for de novo synthesis are well-known in the art and can be adapted for particular applications. Exemplary methods include, for example, chemical synthesis using suitable protecting groups such as CEM (Masuda et al., (2007) Nucleic Acids Symposium Series 51:3-4), the β-cyanoethyl phosphoramidite method (Beaucage S L et al. (1981) Tetrahedron Lett 22:1859); nucleoside H-phosphonate method (Garegg P et al. (1986) Tetrahedron Lett 27:4051-4; Froehler B C et al. (1986) Nucl Acid Res 14:5399-407; Garegg P et al. (1986) Tetrahedron Lett 27:4055-8; Gaffney B L et al. (1988) Tetrahedron Lett 29:2619-22). These chemistries can be performed or adapted for use with automated nucleic acid synthesizers that are commercially available. Additional suitable synthetic methods are disclosed in Uhlmann et al. (1990) Chem Rev 90:544-84, and Goodchild J (1990) Bioconjugate Chem 1: 165. Nucleic acid synthesis can also be performed using suitable recombinant methods that are well-known and conventional in the art, including cloning, processing, and/or expression of polynucleotides and gene products encoded by such polynucleotides. DNA shuffling by random fragmentation and PCR reassembly of gene fragments and synthetic polynucleotides are examples of known techniques that can be used to design and engineer polynucleotide sequences. Site-directed mutagenesis can be used to alter nucleic acids and the encoded proteins, for example, to insert new restriction sites, alter glycosylation patterns, change codon preference, produce splice variants, introduce mutations and the like. Suitable methods for transcription, translation and expression of nucleic acid sequences are known and conventional in the art. (See generally, Current Protocols in Molecular Biology, Vol. 2, Ed. Ausubel, et al., Greene Publish. Assoc. & Wiley Interscience, Ch. 13, 1988; Glover, DNA Cloning, Vol. II, IRL Press, Wash., D.C., Ch. 3, 1986; Bitter, et al., in Methods in Enzymology 153:516-544 (1987); The Molecular Biology of the Yeast Saccharomyces, Eds. Strathern et al., Cold Spring Harbor Press, Vols. I and II, 1982; and Sambrook et al., Molecular Cloning: A Laboratory Manual, Cold Spring Harbor Press, 1989.)

The presence and/or quantity of one or more modified nucleotides in a self-replicating RNA molecule can be determined using any suitable method. For example, a self-replicating RNA can be digested to monophosphates (e.g., using nuclease P1) and dephosphorylated (e.g., using a suitable phosphatase such as CIAP), and the resulting nucleosides analyzed by reversed phase HPLC (e.g., usings a YMC Pack ODS-AQ column (5 micron, 4.6×250 mm) and elute using a gradient, 30% B (0-5 min) to 100% B (5-13 min) and at 100% B (13-40) min, flow Rate (0.7 ml/min), UV detection (wavelength: 260 nm), column temperature (30° C.). Buffer A (20 mM acetic acid—ammonium acetate pH 3.5), buffer B (20 mM acetic acid—ammonium acetate pH 3.5/methanol [90/10])).

Optionally, the self-replicating RNA molecules of the invention may include one or more modified nucleotides so that the self-replicating RNA molecule will have less immunomodulatory activity upon introduction or entry into a host cell (e.g., a human cell) in comparison to the corresponding self-replicating RNA molecule that does not contain modified nucleotides.

If desired, the self-replicating RNA molecules can be screened or analyzed to confirm their therapeutic and prophylactic properties using various in vitro or in vivo testing methods that are known to those of skill in the art. For example, vaccines comprising self-replicating RNA molecule can be tested for their effect on induction of proliferation or effector function of the particular lymphocyte type of interest, e.g., B cells, T cells, T cell lines, and T cell clones. For example, spleen cells from immunized mice can be isolated and the capacity of cytotoxic T lymphocytes to lyse autologous target cells that contain a self replicating RNA molecule that encodes a polypeptide antigen. In addition, T helper cell differentiation can be analyzed by measuring proliferation or production of TH1 (IL-2 and IFN-γ) and/or TH2 (IL-4 and IL-5) cytokines by ELISA or directly in CD4+ T cells by cytoplasmic cytokine staining and flow cytometry.

Self-replicating RNA molecules that encode a polypeptide antigen can also be tested for ability to induce humoral immune responses, as evidenced, for example, by induction of B cell production of antibodies specific for an antigen of interest. These assays can be conducted using, for example, peripheral B lymphocytes from immunized individuals. Such assay methods are known to those of skill in the art. Other assays that can be used to characterize the self-replicating RNA molecules of the invention can involve detecting expression of the encoded antigen by the target cells. For example, FACS can be used to detect antigen expression on the cell surface or intracellularly. Another advantage of FACS selection is that one can sort for different levels of expression; sometimes-lower expression may be desired. Other suitable method for identifying cells which express a particular antigen involve panning using monoclonal antibodies on a plate or capture using magnetic beads coated with monoclonal antibodies.

The self-replicating RNA of the invention may be delivered by a variety of methods, such as naked RNA delivery or in combination with lipids, polymers or other compounds that facilitate entry into the cells. The RNA molecules of the present invention can be introduced into target cells or subjects using any suitable technique, e.g., by direct injection, microinjection, electroporation, lipofection, biolystics, and the like.

C. The Polypeptide Molecule

The immunogenic composition described herein comprises a polypeptide component and an RNA component. The polypeptide component may be a polypeptide complex or a VLP.

Suitable antigens that can be used as the polypeptide component (the “second polypeptide antigen”) of the immunogenic composition include proteins and peptides from any pathogen, such as a bacterial pathogen, a viral pathogen, a fungal pathogen, a protozoan pathogen, or a multi-cellular parasitic pathogen. Exemplary antigens include any one of the antigens described above, such as an antigen derived from RSV, HIV, Parvovirus or CMV. The composition can contain more than one polypeptide antigen. Alternatively or in addition, the polypeptide may also be a fusion polypeptide comprising two or more epitopes from two different proteins of the same pathogen, or two or more epitopes from two different pathogens.

The polypeptide antigen may include additional sequences, such as a sequence to facilitate purification or detection (e.g., a poly-His sequence).

The polypeptide antigen will usually be isolated or purified. Thus, they will not be associated with molecules with which they are normally, if applicable, found in nature.

Polypeptides will usually be prepared by expression in a recombinant host system. Generally, they are produced by expression of recombinant constructs that encode the ecto-domains in suitable recombinant host cells, although any suitable method can be used. Suitable recombinant host cells include, for example, insect cells (e.g., Aedes aegypti, Autographa californica, Bombyx mori, Drosophila melanogaster, Spodoptera frugiperda, and Trichoplusia ni), mammalian cells (e.g., human, non-human primate, horse, cow, sheep, dog, cat, and rodent (e.g., hamster), avian cells (e.g., chicken, duck, and geese), bacteria (e.g., E. coli, Bacillus subtilis, and Streptococcus spp.), yeast cells (e.g., Saccharomyces cerevisiae, Candida albicans, Candida maltosa, Hansenual polymorpha, Kluyveromyces fragilis, Kluyveromyces lactis, Pichia guillerimondii, Pichia pastoris, Schizosaccharomyces pombe and Yarrowia lipolytica), Tetrahymena cells (e.g., Tetrahymena thermophila) or combinations thereof. Many suitable insect cells and mammalian cells are well-known in the art. Suitable insect cells include, for example, Sf9 cells, Sf21 cells, Tn5 cells, Schneider S2 cells, and High Five cells (a clonal isolate derived from the parental Trichoplusia ni BTI-TN-5B1-4 cell line (Invitrogen)). Suitable mammalian cells include, for example, Chinese hamster ovary (CHO) cells, human embryonic kidney cells (HEK293 cells, typically transformed by sheared adenovirus type 5 DNA), NIH-3T3 cells, 293-T cells, Vero cells, HeLa cells, PERC.6 cells (ECACC deposit number 96022940), Hep G2 cells, MRC-5 (ATCC CCL-171), WI-38 (ATCC CCL-75), fetal rhesus lung cells (ATCC CL-160), Madin-Darby bovine kidney (“MDBK”) cells, Madin-Darby canine kidney (“MDCK”) cells (e.g., MDCK (NBL2), ATCC CCL34; or MDCK 33016, DSM ACC 2219), baby hamster kidney (BHK) cells, such as BHK21-F, HKCC cells, and the like. Suitable avian cells include, for example, chicken embryonic stem cells (e.g., EBx® cells), chicken embryonic fibroblasts, chicken embryonic germ cells, duck cells (e.g., AGE1.CR and AGE1.CR.pIX cell lines (ProBioGen) which are described, for example, in Vaccine 27:4975-4982 (2009) and WO2005/042728), EB66 cells, and the like.

Suitable insect cell expression systems, such as baculovirus systems, are known to those of skill in the art and described in, e.g., Summers and Smith, Texas Agricultural Experiment Station Bulletin No. 1555 (1987). Materials and methods for baculovirus/insect cell expression systems are commercially available in kit form from, inter alia, Invitrogen, San Diego CA. Avian cell expression systems are also known to those of skill in the art and described in, e.g., U.S. Pat. Nos. 5,340,740; 5,656,479; 5,830,510; 6,114,168; and 6,500,668; European Patent No. EP 0787180B; European Patent Application No. EP03291813.8; WO 03/043415; and WO 03/076601. Similarly, bacterial and mammalian cell expression systems are also known in the art and described in, e.g., Yeast Genetic Engineering (Barr et al., eds., 1989) Butterworths, London.

Recombinant constructs encoding a polypeptide can be prepared in suitable vectors using conventional methods. A number of suitable vectors for expression of recombinant proteins in insect or mammalian cells are well-known and conventional in the art. Suitable vectors can contain a number of components, including, but not limited to one or more of the following: an origin of replication; a selectable marker gene; one or more expression control elements, such as a transcriptional control element (e.g., a promoter, an enhancer, a terminator), and/or one or more translation signals; and a signal sequence or leader sequence for targeting to the secretory pathway in a selected host cell (e.g., of mammalian origin or from a heterologous mammalian or non-mammalian species). For example, for expression in insect cells a suitable baculovirus expression vector, such as pFastBac (Invitrogen), is used to produce recombinant baculovirus particles. The baculovirus particles are amplified and used to infect insect cells to express recombinant protein. For expression in mammalian cells, a vector that will drive expression of the construct in the desired mammalian host cell (e.g., Chinese hamster ovary cells) is used.

Polypeptides can be purified using any suitable methods. For example, methods for purifying polypeptides by immunoaffinity chromatography are known in the art. Ruiz-Arguello et al., J. Gen. Virol., 85:3677-3687 (2004). Suitable methods for purifying desired proteins including precipitation and various types of chromatography, such as hydrophobic interaction, ion exchange, affinity, chelating and size exclusion are well-known in the art. Suitable purification schemes can be created using two or more of these or other suitable methods. If desired, the polypeptides can include a “tag” that facilitates purification, such as an epitope tag or a HIS tag. Such tagged polypeptides can conveniently be purified, for example from conditioned media, by chelating chromatography or affinity chromatography.

D. Optional RNA Delivery Systems

In addition to the protein compontent and the RNA component, additional components, such as lipids, polymers or other compounds may be optionally included in the immunogenic composition as described herein to facilitate the entry of RNA into target cells.

Although RNA can be delivered as naked RNA (e.g. merely as an aqueous solution of RNA), to enhance entry into cells and also subsequent intercellular effects, the RNA molecule is preferably administered in combination with a delivery system, such as a particulate or emulsion delivery system. A large number of delivery systems are well known to those of skill in the art.

For example, the RNA molecule may be introduced into cells by way of receptor-mediated endocytosis. See e.g., U.S. Pat. No. 6,090,619; Wu and Wu, J. Biol. Chem., 263:14621 (1988); and Curiel et al., Proc. Natl. Acad. Sci. USA, 88:8850 (1991). For example, U.S. Pat. No. 6,083,741 discloses introducing an exogenous nucleic acid into mammalian cells by associating the nucleic acid to a polycation moiety (e.g., poly-L-lysine having 3-100 lysine residues), which is itself coupled to an integrin receptor-binding moiety (e.g., a cyclic peptide having the sequence Arg-Gly-Asp).

The RNA molecule of the present invention can be delivered into cells via amphiphiles. See e.g., U.S. Pat. No. 6,071,890. Typically, a nucleic acid molecule may form a complex with the cationic amphiphile. Mammalian cells contacted with the complex can readily take it up.

Three particularly useful delivery systems are (i) liposomes (ii) non-toxic and biodegradable polymer microparticles (iii) cationic submicron oil-in-water emulsions.

1. Liposomes

Various amphiphilic lipids can form bilayers in an aqueous environment to encapsulate a RNA-containing aqueous core as a liposome. These lipids can have an anionic, cationic or zwitterionic hydrophilic head group. Formation of liposomes from anionic phospholipids dates back to the 1960s, and cationic liposome-forming lipids have been studied since the 1990s. Some phospholipids are anionic whereas other are zwitterionic. Suitable classes of phospholipid include, but are not limited to, phosphatidylethanolamines, phosphatidylcholines, phosphatidylserines, and phosphatidylglycerols, and some useful phospholipids are listed in Table 2. Useful cationic lipids include, but are not limited to, dioleoyl trimethylammonium propane (DOTAP), 1,2-distearyloxy-N,N-dimethyl-3-aminopropane (DSDMA), 1,2-dioleyloxy-N,Ndimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA). Zwitterionic lipids include, but are not limited to, acyl zwitterionic lipids and ether zwitterionic lipids. Examples of useful zwitterionic lipids are DPPC, DOPC and dodecylphosphocholine. The lipids can be saturated or unsaturated.

TABLE 2
Phospholipids

| | |
|---|---|
| DDPC | 1,2-Didecanoyl-sn-Glycero-3-phosphatidylcholine |
| DEPA | 1,2-Dierucoyl-sn-Glycero-3-Phosphate |
| DEPC | 1,2-Erucoyl-sn-Glycero-3-phosphatidylcholine |
| DEPE | 1,2-Dierucoyl-sn-Glycero-3-phosphatidylethanolamine |
| DEPG | 1,2-Dierucoyl-sn-Glycero-3[Phosphatidyl-rac-(1-glycerol. . . ) |
| DLOPC | 1,2-Linoleoyl-sn-Glycero-3-phosphatidylcholine |
| DLPA | 1,2-Dilauroyl-sn-Glycero-3-Phosphate |
| DLPC | 1,2-Dilauroyl-sn-Glycero-3-phosphatidylcholine |
| DLPE | 1,2-Dilauroyl-sn-Glycero-3-phosphatidylethanolamine |
| DLPG | 1,2-Dilauroyl-sn-Glycero-3[Phosphatidyl-rac-(1-glycerol ... ) |
| DLPS | 1,2-Dilauroyl-sn-Glycero-3-phosphatidylserine |
| DMG | 1,2-Dimyristoyl-sn-glycero-3-phosphoethanolamine |
| DMPA | 1,2-Dimyristoyl-sn-Glycero-3-Phosphate |
| DMPC | 1,2-Dimyristoyl-sn-Glycero-3-phosphatidylcholine |
| DMPE | 1,2-Dimyristoyl-sn-Glycero-3-phosphatidylethanolamine |
| DMPG | 1,2-Myristoyl-sn-Glycero-3[Phosphatidyl-rac-(1-glycerol ... ) |
| DMPS | 1,2-Dimyristoyl-sn-Glycero-3-phosphatidylserine |
| DOPA | 1,2-Dioleoyl-sn-Glycero-3-Phosphate |
| DOPC | 1,2-Dioleoyl-sn-Glycero-3-phosphatidylcholine |
| DOPE | 1,2-Dioleoyl-sn-Glycero-3-phosphatidylethanolamine |
| DOPG | 1,2-Dioleoyl-sn-Glycero-3[Phosphatidyl-rac-(1-glycerol ... ) |
| DOPS | 1,2-Dioleoyl-sn-Glycero-3-phosphatidylserine |
| DPPA | 1,2-Dipalmitoyl-sn-Glycero-3-Phosphate |
| DPPC | 1,2-Dipalmitoyl-sn-Glycero-3-phosphatidylcholine |
| DPPE | 1,2-Dipalmitoyl-sn-Glycero-3-phosphatidylethanolamine |
| DPPG | 1,2-Dipalmitoyl-sn-Glycero-3[Phosphatidyl-rac-(1-glycerol ... ) |
| DPPS | 1,2-Dipalmitoyl-sn-Glycero-3-phosphatidylserine |
| DPyPE | 1,2-diphytanoyl-sn-glycero-3-phosphoethanolamine |
| DSPA | 1,2-Distearoyl-sn-Glycero-3-Phosphate |
| DSPC | 1,2-Distearoyl-sn-Glycero-3-phosphatidylcholine |
| DSPE | 1,2-Diostearpyl-sn-Glycero-3-phosphatidylethanolamine |
| DSPG | 1,2-Distearoyl-sn-Glycero-3[Phosphatidyl-rac-(1-glycerol ... ) |
| DSPS | 1,2-Distearoyl-sn-Glycero-3-phosphatidylserine |
| EPC | Egg-PC |
| HEPC | Hydrogenated Egg PC |
| HSPC | High purity Hydrogenated Soy PC |
| HSPC | Hydrogenated Soy PC |
| LYSOPC MYRISTIC | 1-Myristoyl-sn-Glycero-3-phosphatidylcholine |
| LYSOPC PALMITIC | 1-Palmitoyl-sn-Glycero-3-phosphatidylcholine |
| LYSOPC STEARIC | 1-Stearoyl-sn-Glycero-3-phosphatidylcholine |
| Milk Sphingomyelin MPPC | 1-Myristoyl,2-palmitoyl-sn-Glycero 3-phosphatidylcholine |
| MSPC | 1-Myristoyl,2-stearoyl-sn-Glycero-3-phosphatidylcholine |
| PMPC | 1-Palmitoyl,2-myristoyl-sn-Glycero-3-phosphatidylcholine |
| POPC | 1-Palmitoyl,2-oleoyl-sn-Glycero-3-phosphatidylcholine |
| POPE | 1-Palmitoyl-2-oleoyl-sn-Glycero-3-phosphatidylethanolamine |
| POPG | 1,2-Dioleoyl-sn-Glycero-3[Phosphatidyl-rac-(1-glycerol). . . ] |
| PSPC | 1-Palmitoyl,2-stearoyl-sn-Glycero-3-phosphatidylcholine |
| SMPC | 1-Stearoyl,2-myristoyl-sn-Glycero-3-phosphatidylcholine |
| SOPC | 1-Stearoyl,2-oleoyl-sn-Glycero-3-phosphatidylcholine |
| SPPC | 1-Stearoyl,2-palmitoyl-sn-Glycero-3-phosphatidylcholine |

Liposomes can be formed from a single lipid or from a mixture of lipids. A mixture may comprise (i) a mixture of anionic lipids (ii) a mixture of cationic lipids (iii) a mixture of zwitterionic lipids (iv) a mixture of anionic lipids and cationic lipids (v) a mixture of anionic lipids and zwitterionic lipids (vi) a mixture of zwitterionic lipids and cationic lipids or (vii) a mixture of anionic lipids, cationic lipids and zwitterionic lipids. Similarly, a mixture may comprise both saturated and unsaturated lipids. For example, a mixture may comprise DSPC (zwitterionic, saturated), DlinDMA (cationic, unsaturated), and/or DMPG (anionic, saturated). Where a mixture of lipids is used, not all of the component lipids in the mixture need to be amphiphilic e.g. one or more amphiphilic lipids can be mixed with cholesterol.

The hydrophilic portion of a lipid can be PEGylated (i.e. modified by covalent attachment of a polyethylene glycol). This modification can increase stability and prevent non-specific adsorption of the liposomes. For instance, lipids can be conjugated to PEG using techniques such as those disclosed in Heyes et al. (2005) J Controlled Release 107:276-87.

A mixture of DSPC, DlinDMA, PEG-DMPG and cholesterol is used in the examples. A separate aspect of the invention is a liposome comprising DSPC, DlinDMA, PEG-DMG and cholesterol. This liposome preferably encapsulates RNA, such as a self-replicating RNA e.g. encoding an immunogen.

Liposomes are usually divided into three groups: multilamellar vesicles (MLV); small unilamellar vesicles (SUV); and large unilamellar vesicles (LUV). MLVs have multiple bilayers in each vesicle, forming several separate aqueous compartments. SUVs and LUVs have a single bilayer encapsulating an aqueous core; SUVs typically have a diameter ≤50 nm, and LUVs have a diameter >50 nm. Liposomes useful with of the invention are ideally LUVs with a diameter in the range of 50-220 nm. For a composition comprising a population of LUVs with different diameters: (i) at least 80% by number should have diameters in the range of 20-220 nm, (ii) the average diameter (Zav, by intensity) of the population is ideally in the range of 40-200 nm, and/or (iii) the diameters should have a polydispersity index <0.2.

Techniques for preparing suitable liposomes are well known in the art e.g. see Liposomes: Methods and Protocols, Volume 1: Pharmaceutical Nanocarriers: Methods and Protocols. (ed. Weissig). Humana Press, 2009. ISBN 160327359X; Liposome Technology, volumes I, II & III. (ed. Gregoriadis). Informa Healthcare, 2006; and Functional Polymer Colloids and Microparticles volume 4 (Microspheres, microcapsules & liposomes). (eds. Arshady & Guyot). Citus Books, 2002. One useful method involves mixing (i) an ethanolic solution of the lipids (ii) an aqueous solution of the nucleic acid and (iii) buffer, followed by mixing, equilibration, dilution and purification (Heyes et al. (2005) J Controlled Release 107:276-87).

RNA is preferably encapsulated within the liposomes, and so the liposome forms a outer layer around an aqueous RNA-containing core. This encapsulation has been found to protect RNA from RNase digestion. The liposomes can include some external RNA (e.g. on the surface of the liposomes), but at least half of the RNA (and ideally all of it) is encapsulated.

2. Polymeric Microparticles

Various polymers can form microparticles to encapsulate or adsorb RNA. The use of a substantially non-toxic polymer means that a recipient can safely receive the particles, and the use of a biodegradable polymer means that the particles can be metabolised after delivery to avoid long-term persistence. Useful polymers are also sterilisable, to assist in preparing pharmaceutical grade formulations.

Suitable non-toxic and biodegradable polymers include, but are not limited to, poly(α-hydroxy acids), polyhydroxy butyric acids, polylactones (including polycaprolactones), polydioxanones, polyvalerolactone, polyorthoesters, polyanhydrides, polycyanoacrylates, tyrosine-derived polycarbonates, polyvinyl-pyrrolidinones or polyester-amides, and combinations thereof.

In some embodiments, the microparticles are formed from poly(α-hydroxy acids), such as a poly(lactides) (“PLA”), copolymers of lactide and glycolide such as a poly(D,L-lactide-co-glycolide) (“PLG”), and copolymers of D,L-lactide and caprolactone. Useful PLG polymers include those having a lactide/glycolide molar ratio ranging, for example, from 20:80 to 80:20 e.g. 25:75, 40:60, 45:55, 55:45, 60:40, 75:25. Useful PLG polymers include those having a molecular weight between, for example, 5,000-200,000 Da e.g. between 10,000-100,000, 20,000-70,000, 40,000-50,000 Da.

The microparticles ideally have a diameter in the range of 0.02 μm to 8 μm. For a composition comprising a population of microparticles with different diameters at least 80% by number should have diameters in the range of 0.03-7 μm.

Techniques for preparing suitable microparticles are well known in the art e.g. see Functional Polymer Colloids and Microparticles volume 4 (Microspheres, microcapsules & liposomes). (eds. Arshady & Guyot). Citus Books, 2002; Polymers in Drug Delivery. (eds. Uchegbu & Schatzlein). CRC Press, 2006. (in particular chapter 7) and Microparticulate Systems for the Delivery of Proteins and Vaccines. (eds. Cohen & Bernstein). CRC Press, 1996. To facilitate adsorption of RNA, a microparticle may include a cationic surfactant and/or lipid e.g. as disclosed in O'Hagan et al. (2001) J Virology75:9037-9043; and Singh et al. (2003) Pharmaceutical Research 20: 247-251. An alternative way of making polymeric microparticles is by molding and curing e.g. as disclosed in WO2009/132206.

Microparticles of the invention can have a zeta potential of between 40-100 mV.

RNA can be adsorbed to the microparticles, and adsorption is facilitated by including cationic materials (e.g. cationic lipids) in the microparticle.

### 3. Oil-In-Water Cationic Emulsions

Oil-in-water emulsions are known for adjuvanting influenza vaccines e.g. the MF59™ adjuvant in the FLUAD™ product, and the AS03 adjuvant in the PREPANDRIX™ product. RNA delivery according to the present invention can utilise an oil-in-water emulsion, provided that the emulsion includes one or more cationic molecules. For instance, a cationic lipid can be included in the emulsion to provide a positive droplet surface to which negatively-charged RNA can attach.

The emulsion comprises one or more oils. Suitable oil(s) include those from, for example, an animal (such as fish) or a vegetable source. The oil is ideally biodegradable (metabolisable) and biocompatible. Sources for vegetable oils include nuts, seeds and grains. Peanut oil, soybean oil, coconut oil, and olive oil, the most commonly available, exemplify the nut oils. Jojoba oil can be used e.g. obtained from the jojoba bean. Seed oils include safflower oil, cottonseed oil, sunflower seed oil, sesame seed oil and the like. In the grain group, corn oil is the most readily available, but the oil of other cereal grains such as wheat, oats, rye, rice, teff, triticale and the like may also be used. 6-10 carbon fatty acid esters of glycerol and 1,2-propanediol, while not occurring naturally in seed oils, may be prepared by hydrolysis, separation and esterification of the appropriate materials starting from the nut and seed oils. Fats and oils from mammalian milk are metabolizable and so may be used. The procedures for separation, purification, saponification and other means necessary for obtaining pure oils from animal sources are well known in the art.

Most fish contain metabolizable oils which may be readily recovered. For example, cod liver oil, shark liver oils, and whale oil such as spermaceti exemplify several of the fish oils which may be used herein. A number of branched chain oils are synthesized biochemically in 5-carbon isoprene units and are generally referred to as terpenoids. Squalene can also be obtained from yeast or other suitable microbes. In some embodiments, Squalene is preferably obtained from non-animal sources, such as from olives, olive oil or yeast. Squalane, the saturated analog to squalene, can also be used. Fish oils, including squalene and squalane, are readily available from commercial sources or may be obtained by methods known in the art.

Other useful oils are the tocopherols, particularly in combination with squalene. Where the oil phase of an emulsion includes a tocopherol, any of the α, β, γ, δ, ε or ξ tocopherols can be used, but α-tocopherols are preferred. D-α-tocopherol and DL-α-tocopherol can both be used. A preferred α-tocopherol is DL-α-tocopherol. An oil combination comprising squalene and a tocopherol (e.g. DL-α-tocopherol) can be used.

Preferred emulsions comprise squalene, a shark liver oil which is a branched, unsaturated terpenoid (C₃₀H₅₀; [(CH₃)₂C[═CHCH₂CH₂C(CH₃)]₂═CHCH₂-]₂; 2,6,10,15,19,23-hexamethyl-2,6,10,14,18,22-tetracosahexaene; CAS RN 7683-64-9).

The oil in the emulsion may comprise a combination of oils e.g. squalene and at least one further oil.

The aqueous component of the emulsion can be plain water (e.g. w.f.i.) or can include further components e.g. solutes. For instance, it may include salts to form a buffer e.g. citrate or phosphate salts, such as sodium salts. Typical buffers include: a phosphate buffer; a Tris buffer; a borate buffer; a succinate buffer; a histidine buffer; or a citrate buffer. A buffered aqueous phase is preferred, and buffers will typically be included in the 5-20 mM range.

The emulsion also includes a cationic lipid. Preferably this lipid is a surfactant so that it can facilitate formation and stabilisation of the emulsion. Useful cationic lipids generally contains a nitrogen atom that is positively charged under physiological conditions e.g. as a tertiary or quaternary amine. This nitrogen can be in the hydrophilic head group of an amphiphilic surfactant. Useful cationic lipids include, but are not limited to: 1,2-dioleoyloxy-3-(trimethylammonio)propane (DOTAP), 3′[N-(N′,N′-Dimethylaminoethane)-carbamoyl]Cholesterol (DC Cholesterol), dimethyldioctadecyl-ammonium (DDA e.g. the bromide), 1,2-Dimyristoyl-3-Trimethyl-AmmoniumPropane (DMTAP), dipalmitoyl(C16:0)trimethyl ammonium propane (DPTAP), distearoyltrimethylammonium propane (DSTAP). Other useful cationic lipids are: benzalkonium chloride (BAK), benzethonium chloride, cetramide (which contains tetradecyltrimethylammonium bromide and possibly small amounts of dedecyltrimethylammonium bromide and hexadecyltrimethyl ammonium bromide), cetylpyridinium chloride (CPC), cetyl trimethylammonium chloride (CTAC), N,N′,N′-polyoxyethylene (10)-N-tallow-1,3-diaminopropane, dodecyltrimethylammonium bromide, hexadecyltrimethyl-ammonium bromide, mixed alkyl-trimethyl-ammonium bromide, benzyldimethyldodecylammonium chloride, benzyldimethylhexadecyl-ammonium chloride, benzyltrimethylammonium methoxide, cetyldimethylethylammonium bromide, dimethyldioctadecyl ammonium bromide (DDAB), methylbenzethonium chloride, decamethonium chloride, methyl mixed trialkyl ammonium chloride, methyl trioctylammonium chloride), N,N-dimethyl-N-[2 (2-methyl-4-(1,1,3,3tetramethylbutyl)-phenoxy]-ethoxy)ethyl]-benzenemetha-naminium chloride (DEBDA), dialkyldimetylammonium salts, [1-(2,3-dioleyloxy)-propyl]-N,N,N,trimethylammonium chloride, 1,2-diacyl-3-(trimethylammonio) propane (acyl group=dimyristoyl, dipalmitoyl, distearoyl, dioleoyl), 1,2-diacyl-3 (dimethylammonio)propane (acyl group=dimyristoyl, dipalmitoyl, distearoyl, dioleoyl), 1,2-dioleoyl-3-(4′-trimethyl-ammonio)butanoyl-sn-glycerol, 1,2-dioleoyl 3-succinyl-sn-glycerol choline ester, cholesteryl (4′-trimethylammonio) butanoate), N-alkyl pyridinium salts (e.g. cetylpyridinium bromide and cetylpyridinium chloride), N-alkylpiperidinium salts, dicationic bolaform electrolytes (C12Me6; C12BU6), dialkylglycetylphosphorylcholine, lysolecithin, L-α dioleoylphosphatidylethanolamine, cholesterol hemisuccinate choline ester, lipopolyamines, including but not limited to dioctadecylamidoglycylspermine (DOGS), dipalmitoyl phosphatidylethanol-amidospermine (DPPES), lipopoly-L (or D)-lysine (LPLL, LPDL), poly (L (or D)-lysine conjugated to N-glutarylphosphatidylethanolamine, didodecyl glutamate ester with pendant amino group (C\hat{ }GluPhCnN), ditetradecyl glutamate ester with pendant amino group (C14GIuCnN+), cationic derivatives of cholesterol, including but not limited to cholesteryl-3 β-oxysuccinamidoethylenetrimethylammonium salt, cholesteryl-3 β-oxysuccinamidoethylene-dimethylamine, cholesteryl-3 β-carboxyamidoethylenetrimethylammonium salt, and cholesteryl-3 β-carboxyamidoethylenedimethylamine. Other useful cationic lipids are described in US 2008/0085870 and US 2008/0057080, which are incorporated herein by reference.

The cationic lipid is preferably biodegradable (metabolisable) and biocompatible.

In addition to the oil and cationic lipid, an emulsion can include a non-ionic surfactant and/or a zwitterionic surfactant. Such surfactants include, but are not limited to: the polyoxyethylene sorbitan esters surfactants (commonly referred to as the Tweens), especially polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); polyoxyethylene fatty ethers derived from lauryl, cetyl, stearyl and oleyl alcohols (known as Brij surfactants), such as triethyleneglycol monolauryl ether (Brij 30); polyoxyethylene-9-lauryl ether; and sorbitan esters (commonly known as the Spans), such as sorbitan trioleate (Span 85) and sorbitan monolaurate. Preferred surfactants for including in the emulsion are polysorbate 80 (Tween 80; polyoxyethylene sorbitan monooleate), Span 85 (sorbitan trioleate), lecithin and Triton X-100.

Mixtures of these surfactants can be included in the emulsion e.g. Tween 80/Span 85 mixtures, or Tween 80/Triton-X100 mixtures. A combination of a polyoxyethylene sorbitan ester such as polyoxyethylene sorbitan monooleate (Tween 80) and an octoxynol such as t-octylphenoxy-polyethoxyethanol (Triton X-100) is also suitable. Another useful combination comprises laureth 9 plus a polyoxyethylene sorbitan ester and/or an octoxynol. Useful mixtures can comprise a surfactant with a HLB value in the range of 10-20 (e.g. polysorbate 80, with a HLB of 15.0) and a surfactant with a HLB value in the range of 1-10 (e.g. sorbitan trioleate, with a HLB of 1.8).

Preferred amounts of oil (% by volume) in the final emulsion are between 2-20% e.g. 5-15%, 6-14%, 7-13%, 8-12%. A squalene content of about 4-6% or about 9-11% is particularly useful.

Preferred amounts of surfactants (% by weight) in the final emulsion are between 0.001% and 8%. For example: polyoxyethylene sorbitan esters (such as polysorbate 80) 0.2 to 4%, in particular between 0.4-0.6%, between 0.45-0.55%, about 0.5% or between 1.5-2%, between 1.8-2.2%, between 1.9-2.1%, about 2%, or 0.85-0.95%, or about 1%; sorbitan esters (such as sorbitan trioleate) 0.02 to 2%, in particular about 0.5% or about 1%; octyl- or nonylphenoxy polyoxyethanols (such as Triton X-100) 0.001 to 0.1%, in particular 0.005 to 0.02%; polyoxyethylene ethers (such as laureth 9) 0.1 to 8%, preferably 0.1 to 10% and in particular 0.1 to 1% or about 0.5%.

The absolute amounts of oil and surfactant, and their ratio, can be varied within wide limits while still forming an emulsion. A skilled person can easily vary the relative proportions of the components to obtain a desired emulsion, but a weight ratio of between 4:1 and 5:1 for oil and surfactant is typical (excess oil).

An important parameter for ensuring immunostimulatory activity of an emulsion, particularly in large animals, is the oil droplet size (diameter). The most effective emulsions have a droplet size in the submicron range. Suitably the droplet sizes will be in the range 50-750 nm. Most usefully the average droplet size is less than 250 nm e.g. less than 200 nm, less than 150 nm. The average droplet size is usefully in the range of 80-180 nm. Ideally, at least 80% (by number) of the emulsion's oil droplets are less than 250 nm in diameter, and preferably at least 90%. Apparatuses for determining the average droplet size in an emulsion, and the size distribution, are commercially available. These typically use the techniques of dynamic light scattering and/or single-particle optical sensing e.g. the Accusizer™ and Nicomp™ series of instruments available from Particle Sizing Systems (Santa Barbara, USA), or the Zetasizer™ instruments from Malvern Instruments (UK), or the Particle Size Distribution Analyzer instruments from Horiba (Kyoto, Japan).

Ideally, the distribution of droplet sizes (by number) has only one maximum i.e. there is a single population of droplets distributed around an average (mode), rather than having two maxima. Preferred emulsions have a polydispersity of <0.4 e.g. 0.3, 0.2, or less.

Suitable emulsions with submicron droplets and a narrow size distribution can be obtained by the use of microfluidisation. This technique reduces average oil droplet size by propelling streams of input components through geometrically fixed channels at high pressure and high velocity. These streams contact channel walls, chamber walls and each other. The results shear, impact and cavitation forces cause a reduction in droplet size. Repeated steps of microfluidisation can be performed until an emulsion with a desired droplet size average and distribution are achieved.

As an alternative to microfluidisation, thermal methods can be used to cause phase inversion. These methods can also provide a submicron emulsion with a tight particle size distribution.

Preferred emulsions can be filter sterilised i.e. their droplets can pass through a 220 nm filter. As well as providing a sterilisation, this procedure also removes any large droplets in the emulsion.

In certain embodiments, the cationic lipid in the emulsion is DOTAP. The cationic oil-in-water emulsion may comprise from about 0.5 mg/ml to about 25 mg/ml DOTAP. For example, the cationic oil-in-water emulsion may comprise DOTAP at from about 0.5 mg/ml to about 25 mg/ml, from about 0.6 mg/ml to about 25 mg/ml, from about 0.7 mg/ml to about 25 mg/ml, from about 0.8 mg/ml to about 25 mg/ml, from about 0.9 mg/ml to about 25 mg/ml, from about 1.0 mg/ml to about 25 mg/ml, from about 1.1 mg/ml to about 25 mg/ml, from about 1.2 mg/ml to about 25 mg/ml, from about 1.3 mg/ml to about 25 mg/ml, from about 1.4 mg/ml to about 25 mg/ml, from about 1.5 mg/ml to about 25 mg/ml, from about 1.6 mg/ml to about 25 mg/ml, from about 1.7 mg/ml to about 25 mg/ml, from about 0.5 mg/ml to about 24 mg/ml, from about 0.5 mg/ml to about 22 mg/ml, from about 0.5 mg/ml to about 20 mg/ml, from about 0.5 mg/ml to about 18 mg/ml, from about 0.5 mg/ml to about 15 mg/ml, from about 0.5 mg/ml to about 12 mg/ml, from about 0.5 mg/ml to about 10 mg/ml, from about 0.5 mg/ml to about 5 mg/ml, from about 0.5 mg/ml to about 2 mg/ml, from about 0.5 mg/ml to about 1.9 mg/ml, from about 0.5 mg/ml to about 1.8 mg/ml, from about 0.5 mg/ml to about 1.7 mg/ml, from about 0.5 mg/ml to about 1.6 mg/ml, from about 0.6 mg/ml to about 1.6 mg/ml, from about 0.7 mg/ml to about 1.6 mg/ml, from about 0.8 mg/ml to about 1.6 mg/ml, about 0.5 mg/ml, about 0.6 mg/ml, about 0.7 mg/ml, about 0.8 mg/ml, about 0.9 mg/ml, about 1.0 mg/ml, about 1.1 mg/ml, about 1.2 mg/ml, about 1.3 mg/ml, about 1.4 mg/ml, about 1.5 mg/ml, about 1.6 mg/ml, about 12 mg/ml, about 18 mg/ml, about 20 mg/ml, about 21.8 mg/ml, about 24 mg/ml, etc. In an exemplary embodiment, the cationic oil-in-water emulsion comprises from about 0.8 mg/ml to about 1.6 mg/ml DOTAP, such as 0.8 mg/ml, 1.2 mg/ml, 1.4 mg/ml or 1.6 mg/ml.

In certain embodiments, the cationic lipid is DC Cholesterol. The cationic oil-in-water emulsion may comprise DC Cholesterol at from about 0.1 mg/ml to about 5 mg/ml DC Cholesterol. For example, the cationic oil-in-water emulsion may comprise DC Cholesterol from about 0.1 mg/ml to about 5 mg/ml, from about 0.2 mg/ml to about 5 mg/ml, from about 0.3 mg/ml to about 5 mg/ml, from about 0.4 mg/ml to about 5 mg/ml, from about 0.5 mg/ml to about 5 mg/ml, from about 0.62 mg/ml to about 5 mg/ml, from about 1 mg/ml to about 5 mg/ml, from about 1.5 mg/ml to about 5 mg/ml, from about 2 mg/ml to about 5 mg/ml, from about 2.46 mg/ml to about 5 mg/ml, from about 3 mg/ml to about 5 mg/ml, from about 3.5 mg/ml to about 5 mg/ml, from about 4 mg/ml to about 5 mg/ml, from about 4.5 mg/ml to about 5 mg/ml, from about 0.1 mg/ml to about 4.92 mg/ml, from about 0.1 mg/ml to about 4.5 mg/ml, from about 0.1 mg/ml to about 4 mg/ml, from about 0.1 mg/ml to about 3.5 mg/ml, from about 0.1 mg/ml to about 3 mg/ml, from about 0.1 mg/ml to about 2.46 mg/ml, from about 0.1 mg/ml to about 2 mg/ml, from about 0.1 mg/ml to about 1.5 mg/ml, from about 0.1 mg/ml to about 1 mg/ml, from about 0.1 mg/ml to about 0.62 mg/ml, about 0.15 mg/ml, about 0.3 mg/ml, about 0.6 mg/ml, about 0.62 mg/ml, about 0.9 mg/ml, about 1.2 mg/ml, about 2.46 mg/ml, about 4.92 mg/ml, etc. In an exemplary embodiment, the cationic oil-in-water emulsion comprises from about 0.62 mg/ml to about 4.92 mg/ml DC Cholesterol, such as 2.46 mg/ml.

In certain embodiments, the cationic lipid is DDA. The cationic oil-in-water emulsion may comprise from about 0.1 mg/ml to about 5 mg/ml DDA. For example, the cationic oil-in-water emulsion may comprise DDA at from about 0.1 mg/ml to about 5 mg/ml, from about 0.1 mg/ml to about 4.5 mg/ml, from about 0.1 mg/ml to about 4 mg/ml, from about 0.1 mg/ml to about 3.5 mg/ml, from about 0.1 mg/ml to about 3 mg/ml, from about 0.1 mg/ml to about 2.5 mg/ml, from about 0.1 mg/ml to about 2 mg/ml, from about 0.1 mg/ml to about 1.5 mg/ml, from about 0.1 mg/ml to about 1.45 mg/ml, from about 0.2 mg/ml to about 5 mg/ml, from about 0.3 mg/ml to about 5 mg/ml, from about 0.4 mg/ml to about 5 mg/ml, from about 0.5 mg/ml to about 5 mg/ml, from about 0.6 mg/ml to about 5 mg/ml, from about 0.73 mg/ml to about 5 mg/ml, from about 0.8 mg/ml to about 5 mg/ml, from about 0.9 mg/ml to about 5 mg/ml, from about 1.0 mg/ml to about 5 mg/ml, from about 1.2 mg/ml to about 5 mg/ml, from about 1.45 mg/ml to about 5 mg/ml, from about 2 mg/ml to about 5 mg/ml, from about 2.5 mg/ml to about 5 mg/ml, from about 3 mg/ml to about 5 mg/ml, from about 3.5 mg/ml to about 5 mg/ml, from about 4 mg/ml to about 5 mg/ml, from about 4.5 mg/ml to about 5 mg/ml, about 1.2 mg/ml, about 1.45 mg/ml, etc. Alternatively, the cationic oil-in-water emulsion may comprise DDA at about 20 mg/ml, about 21 mg/ml, about 21.5 mg/ml, about 21.6 mg/ml, about 25 mg/ml. In an exemplary embodiment, the cationic oil-in-water emulsion comprises from about 0.73 mg/ml to about 1.45 mg/ml DDA, such as 1.45 mg/ml.

The RNA molecules of the invention can also be delivered to cells ex vivo, such as cells explanted from an individual patient (e.g., lymphocytes, bone marrow aspirates, tissue biopsy) or universal donor hematopoietic stem cells, followed by re-implantation of the cells into a patient, usually after selection for cells which have been transfected with the RNA molecule. The appropriate amount of cells to deliver to a patient will vary with patient conditions, and desired effect, which can be determined by a skilled artisan. See e.g., U.S. Pat. Nos. 6,054,288; 6,048,524; and 6,048,729. Preferably, the cells used are autologous, i.e., cells obtained from the patient being treated.

E. Adjuvants

In certain embodiments, the immunogenic compositions provided herein include or optionally include one or more immunoregulatory agents such as adjuvants. Exemplary adjuvants include, but are not limited to, a TH1 adjuvant and/or a TH2 adjuvant, further discussed below. In certain embodiments, the adjuvants used in the immunogenic compositions provide herein include, but are not limited to:
1. Mineral-Containing Compositions;
2. Oil Emulsions;
3. Saponin Formulations;
4. Virosomes and Virus-Like Particles;
5. Bacterial or Microbial Derivatives;
6. Bioadhesives and Mucoadhesives;
7. Liposomes;
8. Polyoxyethylene Ether and Polyoxyethylene Ester Formulations;
9. Polyphosphazene (PCPP);
10. Muramyl Peptides;
11. Imidazoquinolone Compounds;
12. Thiosemicarbazone Compounds;
13. Tryptanthrin Compounds;
14. Human Immunomodulators;
15. Lipopeptides;
16. Benzonaphthyridines;
17. Microparticles
18. Immunostimulatory polynucleotide (such as RNA or DNA; e.g., CpG-containing oligonucleotides)

1. Mineral Containing Compositions

Mineral containing compositions suitable for use as adjuvants include mineral salts, such as aluminum salts and calcium salts. The immunogenic composition may include mineral salts such as hydroxides (e.g., oxyhydroxides), phosphates (e.g., hydroxyphosphates, orthophosphates), sulfates, etc. (see, e.g., VACCINE DESIGN: THE SUBUNIT AND ADJUVANT APPROACH (Powell, M. F. and Newman, MJ. eds.) (New York: Plenum Press) 1995, Chapters 8 and 9), or mixtures of different mineral compounds (e.g. a mixture of a phosphate and a hydroxide adjuvant, optionally with an excess of the phosphate), with the compounds taking any suitable form (e.g. gel, crystalline, amorphous, etc.), and with adsorption to the salt(s) being preferred. The mineral containing compositions may also be formulated as a particle of metal salt (WO 00/23105).

Aluminum salts may be included in vaccines of the invention such that the dose of Al³⁺ is between 0.2 and 1.0 mg per dose.

In certain embodiments, the aluminum based adjuvant is alum (aluminum potassium sulfate (AlK(SO₄)₂), or an alum derivative, such as that formed in-situ by mixing an antigen in phosphate buffer with alum, followed by titration and precipitation with a base such as ammonium hydroxide or sodium hydroxide.

Another aluminum-based adjuvant suitable for use in vaccine formulations is aluminum hydroxide adjuvant (Al(OH)₃) or crystalline aluminum oxyhydroxide (AlOOH), which is an excellent adsorbent, having a surface area of approximately 500 m²/g. Alternatively, the aluminum based adjuvant can be aluminum phosphate adjuvant (AlPO₄) or aluminum hydroxyphosphate, which contains phosphate groups in place of some or all of the hydroxyl groups of aluminum hydroxide adjuvant. Preferred aluminum phosphate adjuvants provided herein are amorphous and soluble in acidic, basic and neutral media.

In certain embodiments, the adjuvant comprises both aluminum phosphate and aluminum hydroxide. In a more particular embodiment, the adjuvant has a greater amount of aluminum phosphate than aluminum hydroxide, such as a ratio of 2:1, 3:1, 4:1, 5:1, 6:1, 7:1, 8:1, 9:1 or greater than 9:1, by weight aluminum phosphate to aluminum hydroxide. In another embodiment, aluminum salts in the vaccine are present at 0.4 to 1.0 mg per vaccine dose, or 0.4 to 0.8 mg per vaccine dose, or 0.5 to 0.7 mg per vaccine dose, or about 0.6 mg per vaccine dose.

Generally, the preferred aluminum-based adjuvant(s), or ratio of multiple aluminum-based adjuvants, such as aluminum phosphate to aluminum hydroxide is selected by optimization of electrostatic attraction between molecules such that the antigen carries an opposite charge as the adjuvant at the desired pH. For example, aluminum phosphate adjuvant (iep=4) adsorbs lysozyme, but not albumin at pH 7.4. Should albumin be the target, aluminum hydroxide adjuvant would be selected (iep=4). Alternatively, pretreatment of aluminum hydroxide with phosphate lowers its isoelectric point, making it a preferred adjuvant for more basic antigens.

2. Oil-Emulsions

Oil-emulsion compositions and formulations suitable for use as adjuvants (with or without other specific immunostimulating agents such as muramyl peptides or bacterial cell wall components) include squalene-water emulsions, such as MF59 (5% Squalene, 0.5% Tween 80, and 0.5% Span 85, formulated into submicron particles using a microfluidizer). See WO 90/14837. See also, Podda (2001) VACCINE 19: 2673-2680; Frey et al. (2003) Vaccine 21:4234-4237. MF59 is used as the adjuvant in the FLUAD™ influenza virus trivalent subunit vaccine.

Particularly preferred oil-emulsion adjuvants for use in the compositions are submicron oil-in-water emulsions. Preferred submicron oil-in-water emulsions for use herein are squalene/water emulsions optionally containing varying amounts of MTP-PE, such as a submicron oil-in-water emulsion containing 4-5% w/v squalene, 0.25-1.0% w/v Tween 80™ (polyoxyethylenesorbitan monooleate), and/or 0.25-1.0% Span 85™ (sorbitan trioleate), and, optionally, N-acetylmuramyl-L-alanyl-D-isogluatminyl-L-alanine-2-(P-2′-dipalmitoyl-SM-glycero-3-huydroxyphosphophoryloxy)-ethylamine (MTP-PE), for example, the submicron oil-in-water emulsion known as “MF59” (WO 90/14837; U.S. Pat. Nos. 6,299,884; 6,451,325; and Ott et al., “MF59-Design and Evaluation of a Safe and Potent Adjuvant for Human Vaccines” in Vaccine Design: The Subunit and Adjuvant Approach (Powell, M. F. and Newman, M J. eds.) (New York: Plenum Press) 1995, pp. 277-296). MF59 contains 4-5% w/v Squalene (e.g. 4.3%), 0.25-0.5% w/v Tween 80™, and 0.5% w/v Span 85™ and optionally contains various amounts of MTP-PE, formulated into submicron particles using a microfluidizer such as Model 110Y microfluidizer (Microfluidics, Newton, MA). For example, MTP-PE may be present in an amount of about 0-500 μg/dose, more preferably 0-250 μg/dose and most preferably, 0-100 μg/dose. As used herein, the term “MF59-0” refers to the above submicron oil-in-water emulsion lacking MTP-PE, while the term MF59-MTP denotes a formulation that contains MTP-PE. For instance, “MF59-100” contains 100 μg MTP-PE per dose, and so on. MF69, another submicron oil-in-water emulsion for use herein, contains 4.3% w/v squalene, 0.25% w/v Tween 80™, and 0.75% w/v Span 85™ and optionally MTP-PE. Yet another submicron oil-in-water emulsion is MF75, also known as SAF, containing 10% squalene, 0.4% Tween 80™, 5% pluronic-blocked polymer L121, and thr-MDP, also microfluidized into a submicron emulsion. MF75-MTP denotes an MF75 formulation that includes MTP, such as from 100-400 μg MTP-PE per dose.

Submicron oil-in-water emulsions, methods of making the same and immunostimulating agents, such as muramyl peptides, for use in the compositions, are described in detail in WO 90/14837; U.S. Pat. Nos. 6,299,884; and 6,451,325.

Complete Freund's adjuvant (CFA) and incomplete Freund's adjuvant (IFA) may also be used as adjuvants in the invention.

3. Other Immunological Adjuvants

Saponins are a heterologous group of sterol glycosides and triterpenoid glycosides that are found in the bark, leaves, stems, roots and even flowers of a wide range of plant species. Saponins isolated from the bark of the Quillaia saponaria Molina tree have been widely studied as adjuvants. Saponins can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs. Saponin adjuvant formulations include STIMULON® adjuvant (Antigenics, Inc., Lexington, MA).

Saponin compositions have been purified using High Performance Thin Layer Chromatography (HP-TLC) and Reversed Phase High Performance Liquid Chromatography (RP-HPLC). Specific purified fractions using these techniques have been identified, including QS7, QS 17, QS 18, QS21, QH-A, QH-B and QH-C. Preferably, the saponin is QS21. A method of production of QS21 is disclosed in U.S. Pat. No. 5,057,540. Saponin formulations may also comprise a sterol, such as cholesterol (see WO 96/33739).

Saponin formulations may include sterols, cholesterols and lipid formulations. Combinations of saponins and cholesterols can be used to form unique particles called Immunostimulating Complexes (ISCOMs). ISCOMs typically also include a phospholipid such as phosphatidylethanolamine or phosphatidylcholine. Any known saponin can be used in ISCOMs. Preferably, the ISCOM includes one or more of Quil A, QHA and QHC. ISCOMs are further described in EP 0 109 942, WO 96/11711 and WO 96/33739. Optionally, the ISCOMS may be devoid of (an) additional detergent(s). See WO 00/07621.

A review of the development of saponin based adjuvants can be found in Barr et al. (1998) ADV. DRUG DEL. REV. 32:247-271. See also Sjolander et al. (1998) ADV. DRUG DEL. REV. 32:321-338.

Virosomes and Virus Like Particles (VLPs) generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, Qβ-phage (such as coat proteins), GA-phage, fr-phage, AP205 phage, and Ty (such as retrotransposon Ty protein pi). VLPs are discussed further in WO 03/024480; WO 03/024481; Niikura et al. (2002) VIROLOGY 293:273-280; Lenz et al. (2001) J. IMMIUNOL. 166(9):5346-5355′ Pinto et al. (2003) J. INFECT. DIS. 188:327-338; and Gerber et al. (200I) J. VIOL. 75(10):4752-4760. Virosomes are discussed further in, for example, Gluck et al. (2002) VACCINE 20:B10-B16. Immunopotentiating reconstituted influenza virosomes (IRIV) are used as the subunit antigen delivery system in the intranasal trivalent INFLEXAL™ product (Mischler and Metcalfe (2002) VACCINE 20 Suppl 5:B17-B23) and the INFLUVAC PLUS™ product.

Bacterial or microbial derivatives suitable for use as adjuvants include, but are not limited to:

(1) Non-toxic derivatives of enterobacterial lipopolysaccharide (LPS): Such derivatives include Monophosphoryl lipid A (MPL) and 3-O-deacylated MPL (3dMPL). 3dMPL is a mixture of 3 De-O-acylated monophosphoryl lipid A with 4, 5 or 6 acylated chains. A preferred “small particle” form of 3 De-O-acylated monophosphoryl lipid A is disclosed in EP 0 689 454. Such “small particles” of 3dMPL are small enough to be sterile filtered through a 0.22 micron membrane (see EP 0 689 454). Other non-toxic LPS derivatives include monophosphoryl lipid A mimics, such as aminoalkyl glucosaminide phosphate derivatives, e.g., RC-529. See Johnson et al. (1999) Bioorg. Med. Chem. Lett. 9:2273-2278.

(2) Lipid A Derivatives: Lipid A derivatives include derivatives of lipid A from Escherichia coli such as OM-174. OM-174 is described for example in Meraldi et al. (2003) Vaccine 21:2485-2491; and Pajak et al. (2003) Vaccine 21:836-842. Another exemplary adjuvant is the synthetic phospholipid dimer, E6020 (Eisai Co. Ltd., Tokyo, Japan), which mimics the physicochemical and biological properties of many of the natural lipid A's derived from Gram-negative bacteria.

(3) Immunostimulatory oligonucleotides: Immunostimulatory oligonucleotides or polymeric molecules suitable for use as adjuvants in the invention include nucleotide sequences containing a CpG motif (a sequence containing an unmethylated cytosine followed by guanosine and linked by a phosphate bond). Bacterial double stranded RNA or oligonucleotides containing palindromic or poly(dG) sequences have also been shown to be immunostimulatory. The CpG ‘s can include nucleotide modifications/analogs such as phosphorothioate modifications and can be double-stranded or single-stranded. Optionally, the guanosine may be replaced with an analog such as 2’-deoxy-7-deazaguanosine. See Kandimalla et al. (2003) Nucl. Acids Res. 31(9): 2393-2400; WO 02/26757; and WO 99/62923 for examples of possible analog substitutions. The adjuvant effect of CpG oligonucleotides is further discussed in Krieg (2003) Nat. Med. 9(7):831-835; McCluskie et al. (2002) FEMS Immunol. Med. Microbiol. 32: 179-185; WO 98/40100; U.S. Pat. Nos. 6,207,646; 6,239,116; and 6,429,199.

The CpG sequence may be directed to TLR9, such as the motif GTCGTT or TTCGTT. See Kandimalla et al. (2003) Biochem. Soc. Trans. 31 (part 3):654-658. The CpG sequence may be specific for inducing a ThI immune response, such as a CpG-A ODN, or it may be more specific for inducing a B cell response, such a CpG-B ODN. CpG-A and CpG-B ODNs are discussed in Blackwell et al. (2003) J. Immunol. 170(8):4061-4068; Krieg (2002) TRENDS Immunol. 23(2): 64-65; and WO 01/95935. Preferably, the CpG is a CpG-A ODN.

Preferably, the CpG oligonucleotide is constructed so that the 5′ end is accessible for receptor recognition. Optionally, two CpG oligonucleotide sequences may be attached at their 3′ ends to form “immunomers”. See, for example, Kandimalla et al. (2003) BBRC 306:948-953; Kandimalla et al. (2003) Biochem. Soc. Trans. 3 l(part 3):664-658′ Bhagat et al. (2003) BBRC 300:853-861; and WO03/035836.

Immunostimulatory oligonucleotides and polymeric molecules also include alternative polymer backbone structures such as, but not limited to, polyvinyl backbones (Pitha et al. (1970) Biochem. Biophys. Acta 204(1):39-48; Pitha et al. (1970) Biopolymers 9(8):965-977), and morpholino backbones (U.S. Pat. Nos. 5,142,047; 5,185,444). A variety of other charged and uncharged polynucleotide analogs are known in the art. Numerous backbone modifications are known in the art, including, but not limited to, uncharged linkages (e.g., methyl phosphonates, phosphotriesters, phosphoamidates, and carbamates) and charged linkages (e.g., phosphorothioates and phosphorodithioates).

Adjuvant IC31, Intercell AG, Vienna, Austria, is a synthetic formulation that contains an antimicrobial peptide, KLK, and an immunostimulatory oligonucleotide, ODNIa. The two component solution may be simply mixed with antigens (e.g., particles in accordance with the invention with an associated antigen), with no conjugation required.

ADP-ribosylating toxins and detoxified derivatives thereof: Bacterial ADP-ribosylating toxins and detoxified derivatives thereof may be used as adjuvants in the invention. Preferably, the protein is derived from E. coli (i.e., E. coli heat labile enterotoxin “LT”), cholera (“CT”), or pertussis (“PT”). The use of detoxified ADP-ribosylating toxins as mucosal adjuvants is described in WO 95/17211 and as parenteral adjuvants in WO 98/42375. Preferably, the adjuvant is a detoxified LT mutant such as LT-K63, LT-R72, and LTR192G. The use of ADP-ribosylating toxins and detoxified derivatives thereof, particularly LT-K63 and LT-R72, as adjuvants can be found in the following references: Beignon et al. (2002) Infect. Immun. 70(6):3012-3019; Pizza et al. (2001) Vaccine 19:2534-2541; Pizza et al. (2000) J. Med. Microbiol. 290(4-5):455-461; Scharton-Kersten et al. (2000) Infect. Immun. 68(9):5306-5313′ Ryan et al. (1999) Infect. Immun. 67(12):6270-6280; Partidos et al. (1999) Immunol. Lett. 67(3):209-216; Peppoloni et al. (2003) Vaccines 2(2):285-293; and Pine et al. (2002) J. Control Release 85(1-3):263-270. Numerical reference for amino acid substitutions is preferably based on the alignments of the A and B subunits of ADP-ribosylating toxins set forth in Domenighini et al. (1995) MoI. Microbiol. 15(6): 1165-1167.

Bioadhesives and mucoadhesives may also be used as adjuvants. Suitable bioadhesives include esterified hyaluronic acid microspheres (Singh et al. (2001) J. Cont. Release 70:267-276) or mucoadhesives such as cross-linked derivatives of polyacrylic acid, polyvinyl alcohol, polyvinyl pyrollidone, polysaccharides and carboxymethylcellulose. Chitosan and derivatives thereof may also be used as adjuvants in the invention (see WO 99/27960).

Examples of liposome formulations suitable for use as adjuvants are described in U.S. Pat. Nos. 6,090,406; 5,916,588; and EP Patent Publication No. EP 0 626 169.

Adjuvants suitable for use in the invention include polyoxyethylene ethers and polyoxyethylene esters (see, e.g., WO 99/52549). Such formulations further include polyoxyethylene sorbitan ester surfactants in combination with an octoxynol (WO 01/21207) as well as polyoxyethylene alkyl ethers or ester surfactants in combination with at least one additional non-ionic surfactant such as an octoxynol (WO 01/21152). Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

PCPP formulations suitable for use as adjuvants are described, for example, in Andrianov et al. (1998) Biomaterials 19(1-3): 109-115; and Payne et al. (1998) Adv. Drug Del. Rev. 31(3): 185-196.

Examples of muramyl peptides suitable for use as adjuvants include N-acetyl-muramyl-L-threonyl-D-isoglutamine (thr-MDP), N-acetyl-normuramyl-1-alanyl-d-isoglutamine (nor-MDP), and N-acetylmuramyl-1-alanyl-d-isoglutaminyl-1-alanine-2-(1′-2′-dipalmitoyl-sn-glycero-3-hydroxyphosphoryloxy)-ethylamine MTP-PE).

Examples of imidazoquinoline compounds suitable for use as adjuvants include Imiquimod and its analogues, which are described further in Stanley (2002) Clin. Exp. Dermatol. 27(7):571-577; Jones (2003) Curr. Opin. Investig. Drugs 4(2):214-218; and U.S. Pat. Nos. 4,689,338; 5,389,640; 5,268,376; 4,929,624; 5,266,575; 5,352,784; 5,494,916; 5,482,936; 5,346,905; 5,395,937; 5,238,944; and 5,525,612.

Examples of thiosemicarbazone compounds suitable for use as adjuvants, as well as methods of formulating, manufacturing, and screening for such compounds, include those described in WO 04/60308. The thiosemicarbazones are particularly effective in the stimulation of human peripheral blood mononuclear cells for the production of cytokines, such as TNF-α.

Examples of tryptanthrin compounds suitable for use as adjuvants, as well as methods of formulating, manufacturing, and screening for such compounds, include those described in WO 04/64759. The tryptanthrin compounds are particularly effective in the stimulation of human peripheral blood mononuclear cells for the production of cytokines, such as TNF-α. examples of benzonaphthyridine compounds suitable for use as adjuvants include:

Examples of benzonaphthyridine compounds suitable for use as adjuvants, as well as methods of formulating and manufacturing, include those described in WO 2009/111337.

Lipopeptides suitable for use as adjuvants are described above. Other exemplary lipopeptides include, e.g., LP 40, which is an agonist of TLR2. See, e.g., Akdis, et al, EUR. J. IMMUNOLOGY, 33: 2717-26 (2003). Murein lipopeptides are lipopeptides derived from E. coli. See, Hantke, et al., Eur. J. Biochem., 34: 284-296 (1973). Murein lipopeptides comprise a peptide linked to N-acetyl muramic acid, and are thus related to Muramyl peptides, which are described in Baschang, et al., Tetrahedron, 45(20): 6331-6360 (1989).

The human immunomodulators suitable for use as adjuvants include, but are not limited to, cytokines, such as, by way of example only, interleukins (IL-1, IL-2, IL-4, IL-5, IL-6, IL-7, IL-12), interferons (such as, by way of example only, interferon-7), macrophage colony stimulating factor, and tumor necrosis factor.

Microparticles suitable for use as adjuvants include, but are not limited to, microparticles formed from materials that are biodegradable and non-toxic (e.g. a poly(.alpha.-hydroxy acid), a polyhydroxybutyric acid, a polyorthoester, a polyanhydride, a polycaprolactone, etc.), with poly(lactide-co-glycolide). In certain embodiments, such microparticles are treated to have a negatively-charged surface (e.g. with SDS) or a positively-charged surface (e.g. with a cationic detergent, such as CTAB). The microparticles suitable for use as adjuvants have a particle diameter of about 100 nm to about 150 mm in diameter. In certain embodiments, the particle diameter is about 200 nm to about 30 min, and in other embodiments the particle diameter is about 500 nm to 10 min.

### 4. Kits

The invention also provides kits, wherein the RNA molecule encoding the first polypeptide antigen and the second polypeptide antigen are in separate containers. For example, the kit can contain a first container comprising a composition comprising the RNA molecule encoding the first polypeptide antigen, and a second container comprising a composition comprising the second polypeptide antigen.

The kits described may be used for co-delivery of the RNA component and the polypeptide component of the immunogenic compositions described herein (e.g., the RNA component and the polypeptide component may be mixed prior to administration for simultaneous delivery).

The composition that comprises the polypeptide or the RNA molecule can be in liquid form or can be in solid form (e.g., lyophilized). Suitable containers for the compositions include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle).

The kit can further comprise a third container comprising a pharmaceutically-acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery device. The kit may further include a fourth container comprising an adjuvant (such as an aluminum containing adjuvant or MF59).

The kit can also comprise a package insert containing written instructions for methods of inducing immunity or for treating infections. The package insert can be an unapproved draft package insert or can be a package insert approved by the Food and Drug Administration (FDA) or other regulatory body.

The invention also provides a delivery device pre-filled with the immunogenic compositions, the priming compositions, or the boosting compositions described above.

### 5. Immunogenic Compositions

In one aspect, the invention relates to immunogenic compositions comprising: (i) a first polypeptide antigen, and (ii) a self-replicating RNA molecule that encodes a second polypeptide antigen; wherein said first and second polypeptide antigens are from different pathogens.

The immunogenic compositions typically include a pharmaceutically acceptable carrier and/or a suitable delivery system as described herein, such as liposomes, nanoemulsions, PLG micro- and nanoparticles, lipoplexes, chitosan micro- and nanoparticles and other polyplexes. If desired other pharmaceutically acceptable components can be included, such as excipients and adjuvants. These compositions can be used as anti-viral vaccines.

Pharmaceutically acceptable carriers are determined in part by the particular composition being administered, as well as by the particular method used to administer the composition. Accordingly, there is a wide variety of suitable formulations of immunogenic compositions of the present invention. A variety of aqueous carriers can be used. Suitable pharmaceutically acceptable carriers for use in the immunogenic compositions include plain water (e.g. or a buffer e.g. a phosphate buffer, a Tris buffer, a borate buffer, a succinate buffer, a histidine buffer, or a citrate buffer. Buffer salts will typically be included in the 5-20 mM range.

The immunogenic compositions are preferably sterile, and may be sterilized by conventional sterilization techniques.

The compositions may contain pharmaceutically acceptable auxiliary substances as required to approximate physiological conditions such as pH adjusting and buffering agents, and tonicity adjusting agents and the like, for example, sodium acetate, sodium chloride, potassium chloride, calcium chloride, sodium lactate and the like.

Preferably, the immunogenic compositions of the invention may have a pH between 5.0 and 9.5, e.g. between 6.0 and 8.0.

Immunogenic compositions of the invention may include sodium salts (e.g. sodium chloride) to give tonicity. A concentration of 10±2 mg/ml NaCl is typical e.g. about 9 mg/ml.

Immunogenic compositions of the invention may have an osmolality of between 200 mOsm/kg and 400 mOsm/kg, e.g. between 240-360 mOsm/kg, or between 290-310 mOsm/kg.

Immunogenic compositions of the invention may include one or more preservatives, such as thiomersal or 2-phenoxyethanol. Mercury-free compositions are preferred, and preservative-free vaccines can be prepared.

Immunogenic compositions of the invention are preferably non-pyrogenic e.g. containing <1 EU (endotoxin unit, a standard measure) per dose, and preferably <0.1 EU per dose. Immunogenic compositions of the invention are preferably gluten free.

The concentrations of the polypeptide molecule and the RNA molecule in the immunogenic compositions can vary, and will be selected based on fluid volumes, viscosities, body weight and other considerations in accordance with the particular mode of administration selected and the intended recipient's needs. However, the immunogenic compositions are formulated to provide an effective amount of RNA+polypeptide, such as an amount (either in a single dose or as part of a series) that is effective for treatment or prevention. This amount varies depending upon the health and physical condition of the individual to be treated, age, the taxonomic group of individual to be treated (e.g. non-human primate, human primate, etc.), the capacity of the individual's immune system to react to the antigen, the condition to be treated, and other relevant factors. It is expected that the amount will fall in a relatively broad range that can be determined through routine trials. The RNA content of compositions will generally be expressed in terms of the amount of RNA per dose. A preferred dose has ≤200 μg, ≤100 μg, ≤50 μg, or ≤10 μg RNA, and expression can be seen at much lower levels e.g. ≤1 μg/dose, ≤100 ng/dose, ≤10 ng/dose, ≤ing/dose, etc. The amount of polypeptide in each dose will generally comprise from about 0.1 to about 100 μg of polypeptide, with from about 5 to about 50 μg being preferred and from about 5 to about 25 μg/dose being alternatively preferred.

The amount of adjuvant, if any, will be an amount that will induce an immunomodulating response without significant adverse side effect. An optional amount for a particular vaccine can be ascertained by standard studies involving observation of a vaccine's antibody titers and their virus neutralization capabilities. The amount of adjuvant will be from about 1 to about 100 μg/dose, with from about 5 to about 50 μg/dose being preferred, and from about 20 to about 50 μg/dose being alternatively preferred.

Formulations suitable for parenteral administration, such as, for example, by intraarticular (in the joints), intravenous or intraperitoneal injection, and preferably by intramuscular, intradermal or subcutaneous injection, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampoules and vials. Injection solutions and suspensions can be prepared from sterile powders, granules, and tablets. Cells transduced by the RNA molecules can also be administered intravenously or parenterally.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, tragacanth, microcrystalline cellulose, acacia, gelatin, colloidal silicon dioxide, croscarmellose sodium, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, usually sucrose and acacia or tragacanth, as well as pastilles comprising the active ingredient in an inert base, such as gelatin and glycerin or sucrose and acacia emulsions, gels, and the like containing, in addition to the active ingredient, carriers known in the art.

It is recognized that polypeptide and RNA molecules, when administered orally, must be protected from digestion. Protection of polypeptide and RNA molecules can typically be accomplished either by complexing the RNA molecule or the polypeptide molecule with a composition to render the RNA/polypeptide resistant to acidic and enzymatic hydrolysis, or by packaging the RNA molecule or the polypeptide molecule in an appropriately resistant carrier such as a liposome. Means of protecting nucleic acids (such as RNA molecules) and polypeptides from digestion are well known in the art.

The immunogenic compositions can be encapsulated, e.g., in liposomes, or in a formulation that provides for slow release of the active ingredient. For example, the RNA molecule may be formulated as lioposomes, then administered as a priming composition. Alternatively, liposome-formulated RNA may be mixed with the polypeptide molecule to produce the RNA+polypeptide immunogenic composition of the invention. Alternatively, the RNA molecule and the polypeptide molecule can be co-encapsulated in liposomes.

The compositions described herein (immunogenic compositions comprising an RNA and a polypeptide), alone or in combination with other suitable components, can be made into aerosol formulations (e.g., they can be “nebulized”) to be administered via inhalation. Aerosol formulations can be placed into pressurized acceptable propellants, such as dichlorodifluoromethane, propane, nitrogen, and the like.

Suitable suppository formulations may contain the RNA, the polypeptide, or the polypeptide and RNA combination as described herein, and a suppository base. Suitable suppository bases include natural or synthetic triglycerides or paraffin hydrocarbons. It is also possible to use gelatin rectal capsules filled with the polypeptide and RNA molecules as described herein, and a suitable base, for example, liquid triglycerides, polyethylene glycols, and paraffin hydrocarbons.

### 6. Methods of Generating or Enhancing Immune Responses

In another aspect, the invention provides a method for inducing, generating or enhancing an immune response in a subject in need thereof, such as a vertebrate, preferably a mammal, comprising administering an effective amount of an immunogenic composition comprising: (i) a self-replicating RNA molecule that encodes a first polypeptide antigen, and (ii) a second polypeptide antigen; wherein said first and second polypeptide antigens are from different pathogens. The immune response is preferably protective and preferably involves antibodies and/or cell-mediated immunity.

In another aspect, the immunogenic compositions disclosed herein may be used in the manufacture of a medicament for inducing, generating, or enhancing an immune response in a subject in need thereof, such as a vertebrate, preferably a mammal.

In another aspect, the invention provides a method for treating or preventing an infectious disease in a subject (such as a vertebrate, preferably a mammal) in need thereof, comprising administering an effective amount of an immunogenic composition comprising: (i) a self-replicating RNA molecule that encodes a first polypeptide antigen, and (ii) a second polypeptide antigen; wherein said first and second polypeptide antigens are from different pathogens.

In another aspect, the compositions disclosed herein may be used in the manufacture of a medicament for treating or preventing an infectious disease in a subject in need thereof, such as a vertebrate, preferably a mammal.

In another aspect, the invention provides a method for vaccinating a subject, such as a vertebrate, preferably a mammal, or immunizing a subject against a pathogen (e.g., a bacterial pathogen, a viral pathogen, a fungal pathogen, a protozoan pathogen, or a multi-cellular parasitic pathogen), comprising administering to a subject in need thereof an effective amount of an immunogenic composition comprising: (i) a self-replicating RNA molecule that encodes a first polypeptide antigen, and (ii) a second polypeptide antigen; wherein said first and second polypeptide antigens are from different pathogens.

In another aspect, the compositions disclosed herein may be used in the manufacture of a medicament for vaccinating a subject in need thereof, such as a vertebrate, preferably a mammal.

Suitable animal subjects include, for example, fish, birds, cattle, pigs, horses, deer, sheep, goats, bison, rabbits, cats, dogs, chickens, ducks, turkeys, and the like. The mammal is preferably a human. Where the vaccine is for prophylactic use, the human is preferably a child (e.g., a toddler or infant), a teenager, or an adult; where the vaccine is for therapeutic use, the human is preferably a teenager or an adult. A vaccine intended for children may also be administered to adults, e.g., to assess safety, dosage, immunogenicity, etc.

One way of checking efficacy of therapeutic treatment involves monitoring pathogen infection after administration of the compositions or vaccines disclosed herein. One way of checking efficacy of prophylactic treatment involves monitoring immune responses, systemically (such as monitoring the level of IgG1 and IgG2a production) and/or mucosally (such as monitoring the level of IgA production), against the antigen. Typically, antigen-specific serum antibody responses are determined post-immunization but pre-challenge whereas antigen-specific mucosal antibody responses are determined post-immunization and post-challenge.

Another way of assessing the immunogenicity of the compositions or vaccines disclosed herein where the nucleic acid molecule (e.g., the RNA) encodes a protein antigen is to express the protein antigen recombinantly for screening patient sera or mucosal secretions by immunoblot and/or microarrays. A positive reaction between the protein and the patient sample indicates that the patient has mounted an immune response to the protein in question. This method may also be used to identify immunodominant antigens and/or epitopes within protein antigens.

The efficacy of the compositions can also be determined in vivo by challenging appropriate animal models of the pathogen of interest infection.

When the RNA molecule and the polypeptide molecule are co-administered, it may still be desirable to package the polypeptide molecule and RNA molecule separately. The two components may be combined, e.g., within about 72 hours, about 48 hours, about 24 hours, about 12 hours, about 10 hours, about 9 hours, about 8 hours, about 7 hours, about 6 hours, about 5 hours, about 4 hours, about 3 hours, about 2 hours, about 1 hour, about 45 minutes, about 30 minutes, about 15 minutes, about 10 minutes, or about 5 minutes prior to administration. For example, the polypeptide molecule and RNA molecule can be combined at a patient's bedside.

Dosage can be by a single dose schedule or a multiple dose schedule. Multiple doses may be used in a primary immunization schedule and/or in a booster immunization schedule. In a multiple dose schedule the various doses may be given by the same or different routes, e.g., a parenteral prime and mucosal boost, a mucosal prime and parenteral boost, etc. Multiple doses will typically be administered at least 1 week apart (e.g., about 2 weeks, about 3 weeks, about 4 weeks, about 6 weeks, about 8 weeks, about 10 weeks, about 12 weeks, about 16 weeks, etc.). The immunogenic compositions disclosed herein may be used as the prime and/or boost, regardless of whether the immunogenic composition administered as a prime or boost comprised a single pathogen vaccine.

The compositions disclosed herein that include one or more antigens or are used in conjunction with one or more antigens may be used to treat both children and adults. Thus a human subject may be less than 1 year old, 1-5 years old, 5-15 years old, 15-55 years old, or at least 55 years old. Preferred subjects for receiving the compositions are the elderly (e.g., >50 years old, >60 years old, and preferably >65 years), the young (e.g., <5 years old), hospitalized patients, healthcare workers, armed service and military personnel, pregnant women, the chronically ill, or immunodeficient patients. The compositions are not suitable solely for these groups, however, and may be used more generally in a population.

Preferred routes of administration include, but are not limited to, intramuscular, intraperitoneal, intradermal, subcutaneous, intravenous, intraarterial, and intraoccular injection. Oral and transdermal administration, as well as administration by inhalation or suppository is also contemplated. Particularly preferred routes of administration include intramuscular, intradermal and subcutaneous injection. According to some embodiments of the present invention, the composition is administered to a host animal using a needleless injection device, which are well-known and widely available.

It is sometimes advantageous to employ a vaccine that targets a particular target cell type (e.g., an antigen presenting cell or an antigen processing cell).

Catheters or like devices may be used to deliver the composition of the invention, as polypeptide+naked RNA, polypeptide+RNA formulated with a delivery system (e.g., RNA encapsulated in liposomes), RNA only, or polypeptide only into a target organ or tissue. Suitable catheters are disclosed in, e.g., U.S. Pat. Nos. 4,186,745; 5,397,307; 5,547,472; 5,674,192; and 6,129,705, all of which are incorporated herein by reference. The RNA molecules of the invention can also be introduced directly into a tissue, such as muscle. See, e.g., U.S. Pat. No. 5,580,859. Other methods such as “biolistic” or particle-mediated transformation (see, e.g., Sanford et al., U.S. Pat. Nos. 4,945,050; 5,036,006) are also suitable for introduction of RNA into cells of a mammal. These methods are useful not only for in vivo introduction of RNA into a mammal, but also for ex vivo modification of cells for reintroduction into a mammal.

The present invention includes the use of suitable delivery systems, such as liposomes, polymer microparticles or submicron emulsion microparticles with encapsulated or adsorbed RNA, or RNA+polypeptide, to deliver the RNA, or RNA+polypeptide, to elicit an immune response. The invention includes liposomes, microparticles, submicron emulsions, or combinations thereof, with adsorbed and/or encapsulated RNA, or RNA+polypeptide.

The compositions disclosed herein that include one or more antigens, or are used in conjunction with one or more antigens, may be administered to patients at substantially the same time as (e.g., during the same medical consultation or visit to a healthcare professional or vaccination centre) other vaccines, e.g., at substantially the same time as a measles vaccine, a mumps vaccine, a rubella vaccine, a MMR vaccine, a varicella vaccine, a MMRV vaccine, a diphtheria vaccine, a tetanus vaccine, a pertussis vaccine, a DTP vaccine, a conjugated H influenzae type b vaccine, an inactivated poliovirus vaccine, a hepatitis B virus vaccine, a meningococcal conjugate vaccine (such as a tetravalent A C W135 Y vaccine), a respiratory syncytial virus vaccine, etc.

### 7. Definitions

The term “about”, as used herein, refers to +/−10% of a value.

An “antigen” refers to a molecule containing one or more epitopes (either linear, conformational or both).

As used herein, a “polypeptide antigen” refers to a polypeptide comprising one or more epitopes (either linear, conformational or both), that elicits an immunological response. Polypeptide antigens include, for example, a naturally-occurring protein, a mutational variant of a naturally-occurring protein (e.g., a protein that has amino acid substitution(s), addition(s), or deletion(s)), a truncated form of a naturally-occurring protein (e.g., an intracellular domain or extracellular domain of a membrane-anchored protein), as well as a fusion protein (a protein that is derived from at least two different naturally occurring proteins or polypeptide chains). In addition, polypeptide antigens also encompass polypeptides that comprise one or more amino acid stereoisomers, derivatives, or analogues. For example, amino acid derivatives include, e.g., chemical modifications of amino acids such as alkylation, acylation, carbamylation, iodination, etc. Amino acid analogues include, e.g., compounds that have the same basic chemical structure as a naturally occurring amino acid, such as homoserine, norleucine, methionine sulfoxide, methionine methyl sulfonium. Polypeptide antigens also encompass polypeptides that are modified post-translationally (such as acetylated, phosphorylated, or glycosylated polypeptides). Therefore, an epitope of a polypeptide antigen is not limited to a peptide. For example, an epitope of a glycosylated polypeptide may be a saccharide group that is attached to the polypeptide chain.

The term “fusion polypeptide” refers to a single polypeptide in which the amino acid sequence is derived from at least two different naturally occurring proteins or polypeptide chains.

An “epitope” is a portion of an antigen that is recognized by the immune system (e.g., by an antibody, an immunoglobulin receptor, a B cell receptor, or a T cell receptor). An epitope can be linear or conformational. Commonly, an epitope is a polypeptide or polysaccharide in a naturally occurring antigen. In artificial antigens it can be a low molecular weight substance such as an arsanilic acid derivative. Normally, a B-cell epitope will include at least about 5 amino acids but can be as small as 3-4 amino acids. A T-cell epitope, such as a CTL epitope, will typically include at least about 7-9 amino acids, and a helper T-cell epitope will typically include at least about 12-20 amino acids.

When an individual is immunized with a polypeptide antigen having multiple epitopes, in many instances the majority of responding T lymphocytes will be specific for one or a few linear epitopes from that antigen and/or a majority of the responding B lymphocytes will be specific for one or a few linear or conformational epitopes from that antigen. Such epitopes are typically referred to as “immunodominant epitopes.” In an antigen having several immunodominant epitopes, a single epitope may be most dominant, and is typically referred to as the “primary” immunodominant epitope. The remaining immunodominant epitopes are typically referred to as “secondary” immunodominant epitope(s).

As used herein, the terms “minor structural protein” or “minor structural polypeptide” or “minor capsid protein” or “minor capsid polypeptide” or “VP1” in reference to a parvovirus refer to a polypeptide comprising a sequence homologous or identical to the ORF2-encoded polypeptide of a parvovirus, and includes sequences displaying at least about 80-100% sequence identity thereto, including any percent identity within these ranges, such as 81, 82, 83, 84, 85, 86, 87, 88, 89, 90, 91, 92, 93, 94, 95, 96, 97, 98, 99, 100% sequence identity thereto.

As used herein, the terms “major structural protein” or “major structural polypeptide” or “major capsid protein” or “major capsid polypeptide” or “VP2” in reference to a Parvovirus refer to a polypeptide comprising a sequence homologous or identical to the ORF3-encoded polypeptide of a Parvovirus, and include sequences displaying at least about 80-100% sequence identity thereto, including any percent identity within these ranges, such as 81, 82, 83, 84, 85, 86, 87, 88, 89, 90, 91, 92, 93, 94, 95, 96, 97, 98, 99, 100% sequence identity thereto.

The term “naked” as used herein refers to nucleic acids that are substantially free of other macromolecules, such as lipids, polymers, and proteins. A “naked” nucleic acid, such as a self-replicating RNA, is not formulated with other macromolecules to improve cellular uptake. Accordingly, a naked nucleic acid is not encapsulated in, absorbed on, or bound to a liposome, a microparticle or nanoparticle, a cationic emulsion, and the like.

As used herein, “nucleotide analog” or “modified nucleotide” refers to a nucleotide that contains one or more chemical modifications (e.g., substitutions) in or on the nitrogenous base of the nucleoside (e.g., cytosine (C), (thymine (T) or uracil (U)), adenine (A) or guanine (G)). A nucleotide analog can contain further chemical modifications in or on the sugar moiety of the nucleoside (e.g., ribose, deoxyribose, modified ribose, modified deoxyribose, six-membered sugar analog, or open-chain sugar analog), or the phosphate.

As used herein, the term “parvovirus” refers to all parvoviruses associated with mammalian species (e.g., human, canine, chicken, feline, murine, porcine, raccoon, mink, kilham rat, lapine) and broadly to all genus of the Parvoviridae family (i.e., Parvovirus (e.g., canine parvovirus), Dependovirus (e.g., adeno-associated virus), Erythrovirus (e.g., parvovirus B19) and Bocavirus). The term parvovirus also includes isolates not characterized at the time of filing.

As used herein, the term “parvovirus antigen” refers to a molecule derived from a parvovirus, including, without limitation, any of the various isolates of parvovirus. The molecule need not be physically derived from the particular isolate in question, but may be synthetically or recombinantly produced.

The term “pathogen” refers to a virus, eukaryote, prokaryote or archaea that is capable of proliferation, and causes a disease or illness in a host organism, such as a vertebrate (e.g., a mammal). A pathogen can be a viral, bacterial, protozoan, or fungal species, as well as a multi-cellular parasitic species.

The terms “treat,” “treating” or “treatment”, as used herein, include alleviating, abating or ameliorating disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms “treat,” “treating” or “treatment”, include, but are not limited to, prophylactic and/or therapeutic treatments.

The term “viral replicon particle” or “VRP” refers to recombinant infectious virions that cannot generate infectious progeny because of deletion of structural gene(s).

The term “virus-like particle” or “VLP” refers to a structure formed by viral coat proteins (e.g., a capsid) and optionally an evelope, but having no genetic material. A VLP resembles a viral particle.

### EXEMPLIFICATION

The invention now being generally described, it will be more readily understood by reference to the following example, which is included merely for purposes of illustration of certain aspects and embodiments of the present invention, and is not intended to limit the invention.

### Example 1

BALB/c mice, 60 animals total, were divided into 6 groups (10 animals per group). Ten BALB/c mice, per group, were bled before immunization to collect pre-immune sera. Mice were given bilateral intramuscular vaccinations (50 μL per leg) on days 0, 21 and 42 with the formulation indicated in Table 1. The LNP (RV01(15) had the following composition: 40% DlinDMA, 10% DSPC, 48% Chol, 2% PEG DMG 2000 and an N:P ratio of 8:1. Serum was collected for immunological analysis on days 21 (3wp1), 42 (3wp2) and 63 (3wp3).

TABLE 1
Vaccine groups

| Group | Parvovirus | | |
|---|---|---|---|
| # | Antigen | CMV Antigen | Adjuvant |
| 1 | Mutant VP1/VP2 | None | None |
| | VLP, 5 μg | | |
| 2 | Mutant VP1/VP2 | None | MF59 |
| | VLP, 5 μg | | |
| 3 | None | gH full-length/gL | None |
| | | VRP, 1 × 10⁶ IU | |
| 4 | None | gH full-length/gL | LNP (RV01 |
| | | RNA, 1 μg | (15)) |
| 5 | Mutant VP1/VP2 | gH full-length/gL | None |
| | VLP, 5 μg | VRP, 1 × 10⁶ IU | |
| 6 | Mutant VP1/VP2 | gH full-length/gL | LNP (RV01 |
| | VLP, 5 μg | RNA, 1 μg | (15)) |

Three weeks after the immunization the mice were bled again, and sera collected for testing. The following day mice received a second immunization with the same vaccine previously administered to them. Three weeks after the second immunization, the mice were bled and sera collected for testing. A third immunization was administer to the mice the following day. Three weeks after the third immunization, the mice were bled and sera collected for testing.

A Parvovirus specific response was measured by ELISA for serum IgG titers. Wild-type Parvovirus B19 VP1/VP2 was used to coat the microtiter plates. Sera collected from the bleeds (pre-immune, 3 weeks post 1st immunization, and 3 weeks post 2nd immunization) were pooled for each vaccine group (n=10) and measured for IgG titer.

TABLE 2
CMV serum neutralization titers of BALB/c mice, 10 animals per group, after
intramuscular vaccinations on days 0, 21 and 42. Serum was collected for analysis on days
42 (3wp2) and 63 (3wp3) and incubated with TB40 virus on ARPE-19 cells without
complement The neutralization titer is defined as the reciprocal of the serum dilution
producing a 50% reduction in number of positive virus foci per well, relative to controls.

| Vaccine Group | 3wp2 | 3wp3 |
|---|---|---|
| Parvo VLP, 5 mg | <100 | <100 |
| Parvo VLP, 5 mg/MF59 | <100 | <100 |
| CMV VRP, 1E6 | 7,943 | 14,430 |
| CMV RNA, 1 mg/RV01 (35) | 5,497 | 18,328 |
| Parvo VLP + CMV VRP | 12,800 | 17,911 |
| Parvo VLP + CMV RNA/RV01 | 4,569 | 22,688 |

Higher Parvovirus IgG titers were observed in sera from mice who received a vaccine where CMV antigen (either CMV VRP or CMV RNA) was added to Parvovirus VLP vaccine. Three weeks after the first immunization, the IgG titer increased 6-fold (40 v. 250) for Parvovirus+CMV VRP vaccine, as compared to Parvovirus alone vaccine. The difference was even greater (50-fold increase (40 v. 2,000)) three weeks after the first immunization, when the IgG titer of Parvovirus+CMV RNA vaccine was compared to Parvovirus alone vaccine.

Three weeks after the second immunization, the IgG titer increased 2-fold (4,000 v. 9,200) for Parvovirus+CMV VRP vaccine, as compared to Parvovirus alone vaccine. As seen with the first observation, the difference was even greater (35-fold increase (4,000 v. 138,600)) three weeks after the second immunization, when the IgG titer of Parvovirus+CMV RNA vaccine was compared to Parvovirus alone vaccine.

TABLE 3
Parvo-specific serum IgG titers of BALB/c mice, 10 animals per group, 21 (3wp1),
42 (3wp2) and 63 (3wp3) days after intramuscular vaccination on days 0, 21 and 42. Data
are represented as pooled titers of 10 individual mice per group. If an individual animal had a
titer of <25 (limit of detection), it was assigned a titer of 25.

| Serum | | | | |
|---|---|---|---|---|
| Sample | Vaccine group | 3wp1 | 3wp2 | 3wk3 |
| Pre-immune | | <25 | | |
| 1 | Parvo VLP, 5 mg | 41 | 3972 | 404 |
| 2 | Parvo VLP, 5 mg/MF59 | 7868 | 365181 | 53603 |
| 3 | CMV VRP, 1E6 | <25 | <25 | <25 |
| 4 | CMV RNA, 1 mg/RV01 (35) | <25 | <25 | <25 |
| 5 | Parvo VLP + CMV VRP | 246 | 9202 | 1505 |
| 6 | Parvo VLP + CMV RNA/ | 2000 | 138585 | 16649 |
| | RV01 (35) | | | |

The pooled sera were tested for a CMV specific response by measuring serum neutralizing antibody titers. No major change was observed in CMV neutralization titers when Parvovirus VLP was added to either CMV VRP or CMV RNA vaccines.

The highest titers were observed with Parvovirus VLP and MF59 adjuvant. However, the Parvovirus VLP/MF59 composition titer was only ˜3-fold greater than the titer seen when Parvovirus VLP was combined with CMV RNA.

TABLE 4
Parvo serum neutralization titers of BALB/c mice, 10 animals per group,
after intramuscular vaccinations on days 0, 21 and 42. Serum was
collected, pooled for analysis on days 42 (3wp2) and 63 (3wp3) and
tested using an erythroid progenitor cell based qRT-PCR neutralization
assay.

| | | % netralization Titers | | | | |
|---|---|---|---|---|---|---|
| | Dilution | 1:500 | 1:2500 | 1:12500 | 1:62500 | 1:312500 |
| 3wp2 | Parvo VLP/MF59 | 85.96 | 75.57 | 44.66 | 28.03 | −46.09 |
| 3wp2 | Parvo VLP + | 26.90 | 4.10 | 8.27 | 15.78 | −14.01 |
| | CMV RNA/RV01 | | | | | |
| | (35) | | | | | |
| 3wp3 | Parvo VLP | 56.86 | 40.23 | 24.44 | 3.29 | −5.88 |
| 3wp3 | Parvo VLP/MF59 | 94.06 | 87.94 | 70.02 | 37.37 | −19.09 |
| 3wp3 | Parvo VLP + | 73.38 | 44.18 | 19.69 | 9.57 | 15.38 |
| | CMV VRP | | | | | |
| 3wp3 | Parvo VLP + | 83.93 | 73.10 | 40.08 | 32.36 | 25.06 |
| | CMV RNA/RV01 | | | | | |
| | (35) | | | | | |
| 5wp3 | 5 ug Parvo VLP | 89.74 | 80.49 | 58.67 | 57.08 | 25.13 |
| | PI | −229.18 | −191.24 | −94.47 | −112.55 | −197.63 |

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications and patents cited in this disclosure are incorporated by reference in their entirety. To the extent the material incorporated by reference contradicts or is inconsistent with this specification, the specification will supersede any such material. The citation of any references herein is not an admission that such references are prior art to the present invention.

Those skilled in the art will recognize, or be able to ascertain using no more than routine experimentation, many equivalents to the specific embodiments of the invention described herein. Such equivalents are intended to be encompassed by the following embodiments.

SEQUENCES

CMV gB FL: (SEQ ID NO: 1)
1-
atggaaagccggatctggtgcctggtcgtgtgcgtgaacctgtgcatcgtgtgcctgggagc
cgccgtgagcagcagcagcaccagaggcaccagcgccacacacagccaccacagcagccaca
ccacctctgccgcccacagcagatccggcagcgtgtcccagagagtgaccagcagccagacc
gtgtcccacggcgtgaacgagacaatctacaacaccaccctgaagtacggcgacgtcgtggg
cgtgaataccaccaagtacccctacagagtgtgcagcatggcccagggcaccgacctgatca
gattcgagcggaacatcgtgtgcaccagcatgaagcccatcaacgaggacctggacgagggc
atcatggtggtgtacaagagaaacatcgtggcccacaccttcaaagtgcgggtgtaccagaa
ggtgctgaccttccggcggagctacgcctacatccacaccacatacctgctgggcagcaaca
ccgagtacgtggcccctcccatgtgggagatccaccacatcaacagccacagccagtgctac
agcagctacagccgcgtgatcgccggcacagtgttcgtggcctaccaccgggacagctacga
gaacaagaccatgcagctgatgcccgacgactacagcaacacccacagcaccagatacgtga
ccgtgaaggaccagtggcacagcagaggcagcacctggctgtaccgggagacatgcaacctg
aactgcatggtcaccatcaccaccgccagaagcaagtacccttaccacttcttcgccacctc
caccggcgacgtggtggacatcagccccttctacaacggcaccaaccggaacgccagctact
tcggcgagaacgccgacaagttcttcatcttccccaactacaccatcgtgtccgacttcggc
agacccaacagcgctctggaaacccacagactggtggcctttctggaacgggccgacagcgt
gatcagctgggacatccaggacgagaagaacgtgacctgccagctgaccttctgggaggcct
ctgagagaaccatcagaagcgaggccgaggacagctaccacttcagcagcgccaagatgacc
gccaccttcctgagcaagaaacaggaagtgaacatgagcgactccgccctggactgcgtgag
ggacgaggccatcaacaagctgcagcagatcttcaacaccagctacaaccagacctacgaga
agtatggcaatgtgtccgtgttcgagacaacaggcggcctggtggtgttctggcagggcatc
aagcagaaaagcctggtggagctggaacggctcgccaaccggtccagcctgaacctgaccca
caaccggaccaagcggagcaccgacggcaacaacgcaacccacctgtccaacatggaaagcg
tgcacaacctggtgtacgcacagctgcagttcacctacgacaccctgcggggctacatcaac
agagccctggcccagatcgccgaggcttggtgcgtggaccagcggcggaccctggaagtgtt
caaagagctgtccaagatcaaccccagcgccatcctgagcgccatctacaacaagcctatcg
ccgccagattcatgggcgacgtgctgggcctggccagctgcgtgaccatcaaccagaccagc
gtgaaggtgctgcgggacatgaacgtgaaagagagcccaggccgctgctactccagacccgt
ggtcatcttcaacttcgccaacagctcctacgtgcagtacggccagctgggcgaggacaacg
agatcctgctggggaaccaccggaccgaggaatgccagctgcccagcctgaagatctttatc
gccggcaacagcgcctacgagtatgtggactacctgttcaagcggatgatcgacctgagcag
catctccaccgtggacagcatgatcgccctggacatcgaccccctggaaaacaccgacttcc
gggtgctggaactgtacagccagaaagagctgcggagcagcaacgtgttcgacctggaagag
atcatgcgggagttcaacagctacaagcagcgcgtgaaatacgtggaggacaaggtggtgga
ccccctgcctccttacctgaagggcctggacgacctgatgagcggactgggcgctgccggaa
aagccgtgggagtggccattggagctgtgggcggagctgtggcctctgtcgtggaaggcgtc
gccacctttctgaagaaccccttcggcgccttcaccatcatcctggtggccattgccgtcgt
gatcatcacctacctgatctacacccggcagcggagactgtgtacccagcccctgcagaacc
tgttcccctacctggtgtccgccgatggcaccacagtgaccagcggctccaccaaggatacc
agcctgcaggccccacccagctacgaagagagcgtgtacaacagcggcagaaagggccctgg
ccctcccagctctgatgccagcacagccgcccctccctacaccaacgagcaggcctaccaga
tgctgctggccctggctagactggatgccgagcagagggcccagcagaacggcaccgacagc
ctggatggcagaaccggcacccaggacaagggccagaagcccaacctgctggaccggctgcg
gcaccggaagaacggctaccggcacctgaaggacagcgacgaggaagagaacgtctgataa-
2727
CMV gB FL (SEQ ID NO: 2)
MESRIWCLVVCVNLCIVCLGAAVSSSSTRGTSATHSHHSSHTTSAAHSRSGSVSQRVTSSQT
VSHGVNETIYNTTLKYGDVVGVNTTKYPYRVCSMAQGTDLIRFERNIVCTSMKPINEDLDEG
IMVVYKRNIVAHTFKVRVYQKVLTFRRSYAYIHTTYLLGSNTEYVAPPMWEIHHINSHSQCY
SSYSRVIAGTVFVAYHRDSYENKTMQLMPDDYSNTHSTRYVTVKDQWHSRGSTWLYRETCNL
NCMVTITTARSKYPYHFFATSTGDVVDISPFYNGTNRNASYFGENADKFFIFPNYTIVSDFG
RPNSALETHRLVAFLERADSVISWDIQDEKNVTCQLTFWEASERTIRSEAEDSYHFSSAKMT
ATFLSKKQEVNMSDSALDCVRDEAINKLQQIFNTSYNQTYEKYGNVSVFETTGGLVVFWQGI
KQKSLVELERLANRSSLNLTHNRTKRSTDGNNATHLSNMESVHNLVYAQLQFTYDTLRGYIN
RALAQIAEAWCVDQRRTLEVFKELSKINPSAILSAIYNKPIAARFMGDVLGLASCVTINQTS
VKVLRDMNVKESPGRCYSRPVVIFNFANSSYVQYGQLGEDNEILLGNHRTEECQLPSLKIFI
AGNSAYEYVDYLFKRMIDLSSISTVDSMIALDIDPLENTDFRVLELYSQKELRSSNVFDLEE
IMREFNSYKQRVKYVEDKVVDPLPPYLKGLDDLMSGLGAAGKAVGVAIGAVGGAVASVVEGV
ATFLKNPFGAFTIILVAIAVVIITYLIYTRQRRLCTQPLQNLFPYLVSADGTTVTSGSTKDT
SLQAPPSYEESVYNSGRKGPGPPSSDASTAAPPYTNEQAYQMLLALARLDAEQRAQQNGTDS
LDGRTGTQDKGQKPNLLDRLRHRKNGYRHLKDSDEEENV--
CMV gB sol 750: (SEQ ID NO: 3)
1-
atggaaagccggatctggtgcctggtcgtgtgcgtgaacctgtgcatcgtgtgcctgggagc
cgccgtgagcagcagcagcaccagaggcaccagcgccacacacagccaccacagcagccaca
ccacctctgccgcccacagcagatccggcagcgtgtcccagagagtgaccagcagccagacc
gtgtcccacggcgtgaacgagacaatctacaacaccaccctgaagtacggcgacgtcgtggg
cgtgaataccaccaagtacccctacagagtgtgcagcatggcccagggcaccgacctgatca
gattcgagcggaacatcgtgtgcaccagcatgaagcccatcaacgaggacctggacgagggc
atcatggtggtgtacaagagaaacatcgtggcccacaccttcaaagtgcgggtgtaccagaa
ggtgctgaccttccggcggagctacgcctacatccacaccacatacctgctgggcagcaaca
ccgagtacgtggcccctcccatgtgggagatccaccacatcaacagccacagccagtgctac
agcagctacagccgcgtgatcgccggcacagtgttcgtggcctaccaccgggacagctacga
gaacaagaccatgcagctgatgcccgacgactacagcaacacccacagcaccagatacgtga
ccgtgaaggaccagtggcacagcagaggcagcacctggctgtaccgggagacatgcaacctg
aactgcatggtcaccatcaccaccgccagaagcaagtacccttaccacttcttcgccacctc
caccggcgacgtggtggacatcagccccttctacaacggcaccaaccggaacgccagctact
tcggcgagaacgccgacaagttcttcatcttccccaactacaccatcgtgtccgacttcggc
agacccaacagcgctctggaaacccacagactggtggcctttctggaacgggccgacagcgt
gatcagctgggacatccaggacgagaagaacgtgacctgccagctgaccttctgggaggcct
ctgagagaaccatcagaagcgaggccgaggacagctaccacttcagcagcgccaagatgacc
gccaccttcctgagcaagaaacaggaagtgaacatgagcgactccgccctggactgcgtgag
ggacgaggccatcaacaagctgcagcagatcttcaacaccagctacaaccagacctacgaga
agtatggcaatgtgtccgtgttcgagacaacaggcggcctggtggtgttctggcagggcatc
aagcagaaaagcctggtggagctggaacggctcgccaaccggtccagcctgaacctgaccca
caaccggaccaagcggagcaccgacggcaacaacgcaacccacctgtccaacatggaaagcg
tgcacaacctggtgtacgcacagctgcagttcacctacgacaccctgcggggctacatcaac
agagccctggcccagatcgccgaggcttggtgcgtggaccagcggcggaccctggaagtgtt
caaagagctgtccaagatcaaccccagcgccatcctgagcgccatctacaacaagcctatcg
ccgccagattcatgggcgacgtgctgggcctggccagctgcgtgaccatcaaccagaccagc
gtgaaggtgctgcgggacatgaacgtgaaagagagcccaggccgctgctactccagacccgt
ggtcatcttcaacttcgccaacagctcctacgtgcagtacggccagctgggcgaggacaacg
agatcctgctggggaaccaccggaccgaggaatgccagctgcccagcctgaagatctttatc
gccggcaacagcgcctacgagtatgtggactacctgttcaagcggatgatcgacctgagcag
catctccaccgtggacagcatgatcgccctggacatcgaccccctggaaaacaccgacttcc
gggtgctggaactgtacagccagaaagagctgcggagcagcaacgtgttcgacctggaagag
atcatgcgggagttcaacagctacaagcagcgcgtgaaatacgtggaggacaaggtggtgga
ccccctgcctccttacctgaagggcctggacgacctgatgagcggactgggcgctgccggaa
aagccgtgggagtggccattggagctgtgggcggagctgtggcctctgtcgtggaaggcgtc
gccacctttctgaagaactgataa-2256
Cmv gB sol 750 (SEQ ID NO: 4)
MESRIWCLVVCVNLCIVCLGAAVSSSSTRGTSATHSHHSSHTTSAAHSRSGSVSQRVTSSQT
VSHGVNETIYNTTLKYGDVVGVNTTKYPYRVCSMAQGTDLIRFERNIVCTSMKPINEDLDEG
IMVVYKRNIVAHTFKVRVYQKVLTFRRSYAYIHTTYLLGSNTEYVAPPMWEIHHINSHSQCY
SSYSRVIAGTVFVAYHRDSYENKTMQLMPDDYSNTHSTRYVTVKDQWHSRGSTWLYRETCNL
NCMVTITTARSKYPYHFFATSTGDVVDISPFYNGTNRNASYFGENADKFFIFPNYTIVSDFG
RPNSALETHRLVAFLERADSVISWDIQDEKNVTCQLTFWEASERTIRSEAEDSYHFSSAKMT
ATFLSKKQEVNMSDSALDCVRDEAINKLQQIFNTSYNQTYEKYGNVSVFETTGGLVVFWQGI
KQKSLVELERLANRSSLNLTHNRTKRSTDGNNATHLSNMESVHNLVYAQLQFTYDTLRGYIN
RALAQIAEAWCVDQRRTLEVFKELSKINPSAILSAIYNKPIAARFMGDVLGLASCVTINQTS
VKVLRDMNVKESPGRCYSRPVVIFNFANSSYVQYGQLGEDNEILLGNHRTEECQLPSLKIFI
AGNSAYEYVDYLFKRMIDLSSISTVDSMIALDIDPLENTDFRVLELYSQKELRSSNVFDLEE
IMREFNSYKQRVKYVEDKVVDPLPPYLKGLDDLMSGLGAAGKAVGVAIGAVGGAVASVVEGV
ATFLKN--
CMV gB sol 692: (SEQ ID NO: 5)
1-
atggaaagccggatctggtgcctggtcgtgtgcgtgaacctgtgcatcgtgtgcctgggagc
cgccgtgagcagcagcagcaccagaggcaccagcgccacacacagccaccacagcagccaca
ccacctctgccgcccacagcagatccggcagcgtgtcccagagagtgaccagcagccagacc
gtgtcccacggcgtgaacgagacaatctacaacaccaccctgaagtacggcgacgtcgtggg
cgtgaataccaccaagtacccctacagagtgtgcagcatggcccagggcaccgacctgatca
gattcgagcggaacatcgtgtgcaccagcatgaagcccatcaacgaggacctggacgagggc
atcatggtggtgtacaagagaaacatcgtggcccacaccttcaaagtgcgggtgtaccagaa
ggtgctgaccttccggcggagctacgcctacatccacaccacatacctgctgggcagcaaca
ccgagtacgtggcccctcccatgtgggagatccaccacatcaacagccacagccagtgctac
agcagctacagccgcgtgatcgccggcacagtgttcgtggcctaccaccgggacagctacga
gaacaagaccatgcagctgatgcccgacgactacagcaacacccacagcaccagatacgtga
ccgtgaaggaccagtggcacagcagaggcagcacctggctgtaccgggagacatgcaacctg
aactgcatggtcaccatcaccaccgccagaagcaagtacccttaccacttcttcgccacctc
caccggcgacgtggtggacatcagccccttctacaacggcaccaaccggaacgccagctact
tcggcgagaacgccgacaagttcttcatcttccccaactacaccatcgtgtccgacttcggc
agacccaacagcgctctggaaacccacagactggtggcctttctggaacgggccgacagcgt
gatcagctgggacatccaggacgagaagaacgtgacctgccagctgaccttctgggaggcct
ctgagagaaccatcagaagcgaggccgaggacagctaccacttcagcagcgccaagatgacc
gccaccttcctgagcaagaaacaggaagtgaacatgagcgactccgccctggactgcgtgag
ggacgaggccatcaacaagctgcagcagatcttcaacaccagctacaaccagacctacgaga
agtatggcaatgtgtccgtgttcgagacaacaggcggcctggtggtgttctggcagggcatc
aagcagaaaagcctggtggagctggaacggctcgccaaccggtccagcctgaacctgaccca
caaccggaccaagcggagcaccgacggcaacaacgcaacccacctgtccaacatggaaagcg
tgcacaacctggtgtacgcacagctgcagttcacctacgacaccctgcggggctacatcaac
agagccctggcccagatcgccgaggcttggtgcgtggaccagcggcggaccctggaagtgtt
caaagagctgtccaagatcaaccccagcgccatcctgagcgccatctacaacaagcctatcg
ccgccagattcatgggcgacgtgctgggcctggccagctgcgtgaccatcaaccagaccagc
gtgaaggtgctgcgggacatgaacgtgaaagagagcccaggccgctgctactccagacccgt
ggtcatcttcaacttcgccaacagctcctacgtgcagtacggccagctgggcgaggacaacg
agatcctgctggggaaccaccggaccgaggaatgccagctgcccagcctgaagatctttatc
gccggcaacagcgcctacgagtatgtggactacctgttcaagcggatgatcgacctgagcag
catctccaccgtggacagcatgatcgccctggacatcgaccccctggaaaacaccgacttcc
gggtgctggaactgtacagccagaaagagctgcggagcagcaacgtgttcgacctggaagag
atcatgcgggagttcaacagctacaagcagtgataa-2082
Cmv gB sol 692; (SEQ ID NO: 6)
MESRIWCLVVCVNLCIVCLGAAVSSSSTRGTSATHSHHSSHTTSAAHSRSGSVSQRVTSSQT
VSHGVNETIYNTTLKYGDVVGVNTTKYPYRVCSMAQGTDLIRFERNIVCTSMKPINEDLDEG
IMVVYKRNIVAHTFKVRVYQKVLTFRRSYAYIHTTYLLGSNTEYVAPPMWEIHHINSHSQCY
SSYSRVIAGTVFVAYHRDSYENKTMQLMPDDYSNTHSTRYVTVKDQWHSRGSTWLYRETCNL
NCMVTITTARSKYPYHFFATSTGDVVDISPFYNGTNRNASYFGENADKFFIFPNYTIVSDFG
RPNSALETHRLVAFLERADSVISWDIQDEKNVTCQLTFWEASERTIRSEAEDSYHFSSAKMT
ATFLSKKQEVNMSDSALDCVRDEAINKLQQIFNTSYNQTYEKYGNVSVFETTGGLVVFWQGI
KQKSLVELERLANRSSLNLTHNRTKRSTDGNNATHLSNMESVHNLVYAQLQFTYDTLRGYIN
RALAQIAEAWCVDQRRTLEVFKELSKINPSAILSAIYNKPIAARFMGDVLGLASCVTINQTS
VKVLRDMNVKESPGRCYSRPVVIFNFANSSYVQYGQLGEDNEILLGNHRTEECQLPSLKIFI
AGNSAYEYVDYLFKRMIDLSSISTVDSMIALDIDPLENTDFRVLELYSQKELRSSNVFDLEE
IMREFNSYKQ--
CMV gH FL: (SEQ ID NO: 7)
1-
atgaggcctggcctgccctcctacctgatcatcctggccgtgtgcctgttcagccacctgct
gtccagcagatacggcgccgaggccgtgagcgagcccctggacaaggctttccacctgctgc
tgaacacctacggcagacccatccggtttctgcgggagaacaccacccagtgcacctacaac
agcagcctgcggaacagcaccgtcgtgagagagaacgccatcagcttcaactttttccagag
ctacaaccagtactacgtgttccacatgcccagatgcctgtttgccggccctctggccgagc
agttcctgaaccaggtggacctgaccgagacactggaaagataccagcagcggctgaatacc
tacgccctggtgtccaaggacctggccagctaccggtcctttagccagcagctcaaggctca
ggatagcctcggcgagcagcctaccaccgtgccccctcccatcgacctgagcatcccccacg
tgtggatgcctccccagaccacccctcacggctggaccgagagccacaccacctccggcctg
cacagaccccacttcaaccagacctgcatcctgttcgacggccacgacctgctgtttagcac
cgtgaccccctgcctgcaccagggcttctacctgatcgacgagctgagatacgtgaagatca
ccctgaccgaggatttcttcgtggtcaccgtgtccatcgacgacgacacccccatgctgctg
atcttcggccacctgcccagagtgctgttcaaggccccctaccagcgggacaacttcatcct
gcggcagaccgagaagcacgagctgctggtgctggtcaagaaggaccagctgaaccggcact
cctacctgaaggaccccgacttcctggacgccgccctggacttcaactacctggacctgagc
gccctgctgagaaacagcttccacagatacgccgtggacgtgctgaagtccggacggtgcca
gatgctcgatcggcggaccgtggagatggccttcgcctatgccctcgccctgttcgccgctg
ccagacaggaagaggctggcgcccaggtgtcagtgcccagagccctggatagacaggccgcc
ctgctgcagatccaggaattcatgatcacctgcctgagccagaccccccctagaaccaccct
gctgctgtaccccacagccgtggatctggccaagagggccctgtggacccccaaccagatca
ccgacatcacaagcctcgtgcggctcgtgtacatcctgagcaagcagaaccagcagcacctg
atcccccagtgggccctgagacagatcgccgacttcgccctgaagctgcacaagacccatct
ggccagctttctgagcgccttcgccaggcaggaactgtacctgatgggcagcctggtccaca
gcatgctggtgcataccaccgagcggcgggagatcttcatcgtggagacaggcctgtgtagc
ctggccgagctgtcccactttacccagctgctggcccaccctcaccacgagtacctgagcga
cctgtacaccccctgcagcagcagcggcagacgggaccacagcctggaacggctgaccagac
tgttccccgatgccaccgtgcctgctacagtgcctgccgccctgtccatcctgtccaccatg
cagcccagcaccctggaaaccttccccgacctgttctgcctgcccctgggcgagagctttag
cgccctgaccgtgtccgagcacgtgtcctacatcgtgaccaatcagtacctgatcaagggca
tcagctaccccgtgtccaccacagtcgtgggccagagcctgatcatcacccagaccgacagc
cagaccaagtgcgagctgacccggaacatgcacaccacacacagcatcaccgtggccctgaa
catcagcctggaaaactgcgctttctgtcagtctgccctgctggaatacgacgatacccagg
gcgtgatcaacatcatgtacatgcacgacagcgacgacgtgctgttcgccctggacccctac
aacgaggtggtggtgtccagcccccggacccactacctgatgctgctgaagaacggcaccgt
gctggaagtgaccgacgtggtggtggacgccaccgacagcagactgctgatgatgagcgtgt
acgccctgagcgccatcatcggcatctacctgctgtaccggatgctgaaaacctgctgataa-
2232
Cmv gH FL; (SEQ ID NO: 8)
MRPGLPSYLIILAVCLFSHLLSSRYGAEAVSEPLDKAFHLLLNTYGRPIRFLRENTTQCTYN
SSLRNSTVVRENAISFNFFQSYNQYYVFHMPRCLFAGPLAEQFLNQVDLTETLERYQQRLNT
YALVSKDLASYRSFSQQLKAQDSLGEQPTTVPPPIDLSIPHVWMPPQTTPHGWTESHTTSGL
HRPHFNQTCILFDGHDLLFSTVTPCLHQGFYLIDELRYVKITLTEDFFVVTVSIDDDTPMLL
IFGHLPRVLFKAPYQRDNFILRQTEKHELLVLVKKDQLNRHSYLKDPDFLDAALDFNYLDLS
ALLRNSFHRYAVDVLKSGRCQMLDRRTVEMAFAYALALFAAARQEEAGAQVSVPRALDRQAA
LLQIQEFMITCLSQTPPRTTLLLYPTAVDLAKRALWTPNQITDITSLVRLVYILSKQNQQHL
IPQWALRQIADFALKLHKTHLASFLSAFARQELYLMGSLVHSMLVHTTERREIFIVETGLCS
LAELSHFTQLLAHPHHEYLSDLYTPCSSSGRRDHSLERLTRLFPDATVPATVPAALSILSTM
QPSTLETFPDLFCLPLGESFSALTVSEHVSYIVTNQYLIKGISYPVSTTVVGQSLIITQTDS
QTKCELTRNMHTTHSITVALNISLENCAFCQSALLEYDDTQGVINIMYMHDSDDVLFALDPY
NEVVVSSPRTHYLMLLKNGTVLEVTDVVVDATDSRLLMMSVYALSAIIGIYLLYRMLKTC--
CMV gH sol: (SEQ ID NO: 9)
1-
atgaggcctggcctgccctcctacctgatcatcctggccgtgtgcctgttcagccacctgct
gtccagcagatacggcgccgaggccgtgagcgagcccctggacaaggctttccacctgctgc
tgaacacctacggcagacccatccggtttctgcgggagaacaccacccagtgcacctacaac
agcagcctgcggaacagcaccgtcgtgagagagaacgccatcagcttcaactttttccagag
ctacaaccagtactacgtgttccacatgcccagatgcctgtttgccggccctctggccgagc
agttcctgaaccaggtggacctgaccgagacactggaaagataccagcagcggctgaatacc
tacgccctggtgtccaaggacctggccagctaccggtcctttagccagcagctcaaggctca
ggatagcctcggcgagcagcctaccaccgtgccccctcccatcgacctgagcatcccccacg
tgtggatgcctccccagaccacccctcacggctggaccgagagccacaccacctccggcctg
cacagaccccacttcaaccagacctgcatcctgttcgacggccacgacctgctgtttagcac
cgtgaccccctgcctgcaccagggcttctacctgatcgacgagctgagatacgtgaagatca
ccctgaccgaggatttcttcgtggtcaccgtgtccatcgacgacgacacccccatgctgctg
atcttcggccacctgcccagagtgctgttcaaggccccctaccaggggacaacttcatcct
gcggcagaccgagaagcacgagctgctggtgctggtcaagaaggaccagctgaaccggcact
cctacctgaaggaccccgacttcctggacgccgccctggacttcaactacctggacctgagc
gccctgctgagaaacagcttccacagatacgccgtggacgtgctgaagtccggacggtgcca
gatgctcgatcggcggaccgtggagatggccttcgcctatgccctcgccctgttcgccgctg
ccagacaggaagaggctggcgcccaggtgtcagtgcccagagccctggatagacaggccgcc
ctgctgcagatccaggaattcatgatcacctgcctgagccagaccccccctagaaccaccct
gctgctgtaccccacagccgtggatctggccaagagggccctgtggacccccaaccagatca
ccgacatcacaagcctcgtgcggctcgtgtacatcctgagcaagcagaaccagcagcacctg
atcccccagtgggccctgagacagatcgccgacttcgccctgaagctgcacaagacccatct
ggccagctttctgagcgccttcgccaggcaggaactgtacctgatgggcagcctggtccaca
gcatgctggtgcataccaccgagcggcgggagatcttcatcgtggagacaggcctgtgtagc
ctggccgagctgtcccactttacccagctgctggcccaccctcaccacgagtacctgagcga
cctgtacaccccctgcagcagcagcggcagacgggaccacagcctggaacggctgaccagac
tgttccccgatgccaccgtgcctgctacagtgcctgccgccctgtccatcctgtccaccatg
cagcccagcaccctggaaaccttccccgacctgttctgcctgcccctgggcgagagctttag
cgccctgaccgtgtccgagcacgtgtcctacatcgtgaccaatcagtacctgatcaagggca
tcagctaccccgtgtccaccacagtcgtgggccagagcctgatcatcacccagaccgacagc
cagaccaagtgcgagctgacccggaacatgcacaccacacacagcatcaccgtggccctgaa
catcagcctggaaaactgcgctttctgtcagtctgccctgctggaatacgacgatacccagg
gcgtgatcaacatcatgtacatgcacgacagcgacgacgtgctgttcgccctggacccctac
aacgaggtggtggtgtccagcccccggacccactacctgatgctgctgaagaacggcaccgt
gctggaagtgaccgacgtggtggtggacgccaccgactgataa-2151
CMV gH sol; (SEQ ID NO: 10)
MRPGLPSYLIILAVCLFSHLLSSRYGAEAVSEPLDKAFHLLLNTYGRPIRFLRENTTQCTYN
SSLRNSTVVRENAISFNFFQSYNQYYVFHMPRCLFAGPLAEQFLNQVDLTETLERYQQRLNT
YALVSKDLASYRSFSQQLKAQDSLGEQPTTVPPPIDLSIPHVWMPPQTTPHGWTESHTTSGL
HRPHFNQTCILFDGHDLLFSTVTPCLHQGFYLIDELRYVKITLTEDFFVVTVSIDDDTPMLL
IFGHLPRVLFKAPYQRDNFILRQTEKHELLVLVKKDQLNRHSYLKDPDFLDAALDFNYLDLS
ALLRNSFHRYAVDVLKSGRCQMLDRRTVEMAFAYALALFAAARQEEAGAQVSVPRALDRQAA
LLQIQEFMITCLSQTPPRTTLLLYPTAVDLAKRALWTPNQITDITSLVRLVYILSKQNQQHL
IPQWALRQIADFALKLHKTHLASFLSAFARQELYLMGSLVHSMLVHTTERREIFIVETGLCS
LAELSHFTQLLAHPHHEYLSDLYTPCSSSGRRDHSLERLTRLFPDATVPATVPAALSILSTM
QPSTLETFPDLFCLPLGESFSALTVSEHVSYIVTNQYLIKGISYPVSTTVVGQSLIITQTDS
QTKCELTRNMHTTHSITVALNISLENCAFCQSALLEYDDTQGVINIMYMHDSDDVLFALDPY
NEVVVSSPRTHYLMLLKNGTVLEVTDVVVDATD--
CMV gL fl: (SEQ ID NO: 11)
1-
atgtgcagaaggcccgactgcggcttcagcttcagccctggacccgtgatcctgctgtggtg
ctgcctgctgctgcctatcgtgtcctctgccgccgtgtctgtggcccctacagccgccgaga
aggtgccagccgagtgccccgagctgaccagaagatgcctgctgggcgaggtgttcgagggc
gacaagtacgagagctggctgcggcccctggtcaacgtgaccggcagagatggccccctgag
ccagctgatccggtacagacccgtgacccccgaggccgccaatagcgtgctgctggacgagg
ccttcctggataccctggccctgctgtacaacaaccccgaccagctgagagccctgctgacc
ctgctgtccagcgacaccgcccccagatggatgaccgtgatgcggggctacagcgagtgtgg
agatggcagccctgccgtgtacacctgcgtggacgacctgtgcagaggctacgacctgacca
gactgagctacggccggtccatcttcacagagcacgtgctgggcttcgagctggtgcccccc
agcctgttcaacgtggtggtggccatccggaacgaggccaccagaaccaacagagccgtgcg
gctgcctgtgtctacagccgctgcacctgagggcatcacactgttctacggcctgtacaacg
ccgtgaaagagttctgcctccggcaccagctggatccccccctgctgagacacctggacaag
tactacgccggcctgcccccagagctgaagcagaccagagtgaacctgcccgcccacagcag
atatggccctcaggccgtggacgccagatgataa-840
CMV gL FL; (SEQ ID NO: 12)
MCRRPDCGFSFSPGPVILLWCCLLLPIVSSAAVSVAPTAAEKVPAECPELTRRCLLGEVFEG
DKYESWLRPLVNVTGRDGPLSQLIRYRPVTPEAANSVLLDEAFLDTLALLYNNPDQLRALLT
LLSSDTAPRWMTVMRGYSECGDGSPAVYTCVDDLCRGYDLTRLSYGRSIFTEHVLGFELVPP
SLFNVVVAIRNEATRTNRAVRLPVSTAAAPEGITLFYGLYNAVKEFCLRHQLDPPLLRHLDK
YYAGLPPELKQTRVNLPAHSRYGPQAVDAR--
CMV gM FL: (SEQ ID NO: 13)
1-
atggcccccagccacgtggacaaagtgaacacccggacttggagcgccagcatcgtgttcat
ggtgctgaccttcgtgaacgtgtccgtgcacctggtgctgtccaacttcccccacctgggct
acccctgcgtgtactaccacgtggtggacttcgagcggctgaacatgagcgcctacaacgtg
atgcacctgcacacccccatgctgtttctggacagcgtgcagctcgtgtgctacgccgtgtt
catgcagctggtgtttctggccgtgaccatctactacctcgtgtgctggatcaagatcagca
tgcggaaggacaagggcatgagcctgaaccagagcacccgggacatcagctacatgggcgac
agcctgaccgccttcctgttcatcctgagcatggacaccttccagctgttcaccctgaccat
gagcttccggctgcccagcatgatcgccttcatggccgccgtgcactttttctgtctgacca
tcttcaacgtgtccatggtcacccagtaccggtcctacaagcggagcctgttcttcttctcc
cggctgcaccccaagctgaagggcaccgtgcagttccggaccctgatcgtgaacctggtgga
ggtggccctgggcttcaataccaccgtggtggctatggccctgtgctacggcttcggcaaca
acttcttcgtgcggaccggccatatggtgctggccgtgttcgtggtgtacgccatcatcagc
atcatctactttctgctgatcgaggccgtgttcttccagtacgtgaaggtgcagttcggcta
ccatctgggcgcctttttcggcctgtgcggcctgatctaccccatcgtgcagtacgacacct
tcctgagcaacgagtaccggaccggcatcagctggtccttcggaatgctgttcttcatctgg
gccatgttcaccacctgcagagccgtgcggtacttcagaggcagaggcagcggctccgtgaa
gtaccaggccctggccacagcctctggcgaagaggtggccgccctgagccaccacgacagcc
tggaaagcagacggctgcgggaggaagaggacgacgacgacgaggacttcgaggacgcctga
taa-1119
CMV gM FL; (SEQ ID NO: 14)
MAPSHVDKVNTRTWSASIVFMVLTFVNVSVHLVLSNFPHLGYPCVYYHVVDFERLNMSAYNV
MHLHTPMLFLDSVQLVCYAVFMQLVFLAVTIYYLVCWIKISMRKDKGMSLNQSTRDISYMGD
SLTAFLFILSMDTFQLFTLTMSFRLPSMIAFMAAVHFFCLTIFNVSMVTQYRSYKRSLFFFS
RLHPKLKGTVQFRTLIVNLVEVALGFNTTVVAMALCYGFGNNFFVRTGHMVLAVFVVYAIIS
IIYFLLIEAVFFQYVKVQFGYHLGAFFGLCGLIYPIVQYDTFLSNEYRTGISWSFGMLFFIW
AMFTTCRAVRYFRGRGSGSVKYQALATASGEEVAALSHHDSLESRRLREEEDDDDEDFEDA--
CMV gN FL: (SEQ ID NO: 15)
1-
atggaatggaacaccctggtcctgggcctgctggtgctgtctgtcgtggccagcagcaacaa
cacatccacagccagcacccctagacctagcagcagcacccacgccagcactaccgtgaagg
ctaccaccgtggccaccacaagcaccaccactgctaccagcaccagctccaccacctctgcc
aagcctggctctaccacacacgaccccaacgtgatgaggccccacgcccacaacgacttcta
caacgctcactgcaccagccacatgtacgagctgtccctgagcagctttgccgcctggtgga
ccatgctgaacgccctgatcctgatgggcgccttctgcatcgtgctgcggcactgctgcttc
cagaacttcaccgccaccaccaccaagggctactgataa-411
CMV gN FL; (SEQ ID NO: 16)
MEWNTLVLGLLVLSVVASSNNTSTASTPRPSSSTHASTTVKATTVATTSTTTATSTSSTTSA
KPGSTTHDPNVMRPHAHNDFYNAHCTSHMYELSLSSFAAWWTMLNALILMGAFCIVLRHCCF
QNFTATTTKGY--
CMV gO FL: (SEQ ID NO: 17)
1-
atgggcaagaaagaaatgatcatggtcaagggcatccccaagatcatgctgctgattagcat
cacctttctgctgctgtccctgatcaactgcaacgtgctggtcaacagccggggcaccagaa
gatcctggccctacaccgtgctgtcctaccggggcaaagagatcctgaagaagcagaaagag
gacatcctgaagcggctgatgagcaccagcagcgacggctaccggttcctgatgtaccccag
ccagcagaaattccacgccatcgtgatcagcatggacaagttcccccaggactacatcctgg
ccggacccatccggaacgacagcatcacccacatgtggttcgacttctacagcacccagctg
cggaagcccgccaaatacgtgtacagcgagtacaaccacaccgcccacaagatcaccctgag
gcctcccccttgtggcaccgtgcccagcatgaactgcctgagcgagatgctgaacgtgtcca
agcggaacgacaccggcgagaagggctgcggcaacttcaccaccttcaaccccatgttcttc
aacgtgccccggtggaacaccaagctgtacatcggcagcaacaaagtgaacgtggacagcca
gaccatctactttctgggcctgaccgccctgctgctgagatacgcccagcggaactgcaccc
ggtccttctacctggtcaacgccatgagccggaacctgttccgggtgcccaagtacatcaac
ggcaccaagctgaagaacaccatgcggaagctgaagcggaagcaggccctggtcaaagagca
gccccagaagaagaacaagaagtcccagagcaccaccaccccctacctgagctacaccacct
ccaccgccttcaacgtgaccaccaacgtgacctacagcgccacagccgccgtgaccagagtg
gccacaagcaccaccggctaccggcccgacagcaactttatgaagtccatcatggccaccca
gctgagagatctggccacctgggtgtacaccaccctgcggtacagaaacgagcccttctgca
agcccgaccggaacagaaccgccgtgagcgagttcatgaagaatacccacgtgctgatcaga
aacgagacaccctacaccatctacggcaccctggacatgagcagcctgtactacaacgagac
aatgagcgtggagaacgagacagccagcgacaacaacgaaaccacccccacctcccccagca
cccggttccagcggaccttcatcgaccccctgtgggactacctggacagcctgctgttcctg
gacaagatccggaacttcagcctgcagctgcccgcctacggcaatctgaccccccctgagca
cagaagggccgccaacctgagcaccctgaacagcctgtggtggtggagccagtgataa-
1422
CMV gO FL; (SEQ ID NO: 18)
MGKKEMIMVKGIPKIMLLISITFLLLSLINCNVLVNSRGTRRSWPYTVLSYRGKEILKKQKE
DILKRLMSTSSDGYRFLMYPSQQKFHAIVISMDKFPQDYILAGPIRNDSITHMWFDFYSTQL
RKPAKYVYSEYNHTAHKITLRPPPCGTVPSMNCLSEMLNVSKRNDTGEKGCGNFTTFNPMFF
NVPRWNTKLYIGSNKVNVDSQTIYFLGLTALLLRYAQRNCTRSFYLVNAMSRNLFRVPKYIN
GTKLKNTMRKLKRKQALVKEQPQKKNKKSQSTTTPYLSYTTSTAFNVTTNVTYSATAAVTRV
ATSTTGYRPDSNFMKSIMATQLRDLATWVYTTLRYRNEPFCKPDRNRTAVSEFMKNTHVLIR
NETPYTIYGTLDMSSLYYNETMSVENETASDNNETTPTSPSTRFQRTFIDPLWDYLDSLLFL
DKIRNFSLQLPAYGNLTPPEHRRAANLSTLNSLWWWSQ--
CMV UL128 FL: (SEQ ID NO: 19)
1-
atgagccccaaggacctgacccccttcctgacaaccctgtggctgctcctgggccatagcag
agtgcctagagtgcgggccgaggaatgctgcgagttcatcaacgtgaaccacccccccgagc
ggtgctacgacttcaagatgtgcaaccggttcaccgtggccctgagatgccccgacggcgaa
gtgtgctacagccccgagaaaaccgccgagatccggggcatcgtgaccaccatgacccacag
cctgacccggcaggtggtgcacaacaagctgaccagctgcaactacaaccccctgtacctgg
aagccgacggccggatcagatgcggcaaagtgaacgacaaggcccagtacctgctgggagcc
gccggaagcgtgccctaccggtggatcaacctggaatacgacaagatcacccggatcgtggg
cctggaccagtacctggaaagcgtgaagaagcacaagcggctggacgtgtgcagagccaaga
tgggctacatgctgcagtgataa-519
CMV UL128 FL; (SEQ ID NO: 20)
MSPKDLTPFLTTLWLLLGHSRVPRVRAEECCEFINVNHPPERCYDFKMCNRFTVALRCPDGE
VCYSPEKTAEIRGIVTTMTHSLTRQVVHNKLTSCNYNPLYLEADGRIRCGKVNDKAQYLLGA
AGSVPYRWINLEYDKITRIVGLDQYLESVKKHKRLDVCRAKMGYMLQ--
CMV UL130 FL: (SEQ ID NO: 21)
1 -
atgctgcggctgctgctgagacaccacttccactgcctgctgctgtgtgccgtgtgggccac
cccttgtctggccagcccttggagcaccctgaccgccaaccagaaccctagccccccttggt
ccaagctgacctacagcaagccccacgacgccgccaccttctactgcccctttctgtacccc
agccctcccagaagccccctgcagttcagcggcttccagagagtgtccaccggccctgagtg
ccggaacgagacactgtacctgctgtacaaccgggagggccagacactggtggagcggagca
gcacctgggtgaaaaaagtgatctggtatctgagcggccggaaccagaccatcctgcagcgg
atgcccagaaccgccagcaagcccagcgacggcaacgtgcagatcagcgtggaggacgccaa
aatcttcggcgcccacatggtgcccaagcagaccaagctgctgagattcgtggtcaacgacg
gcaccagatatcagatgtgcgtgatgaagctggaaagctgggcccacgtgttccgggactac
tccgtgagcttccaggtccggctgaccttcaccgaggccaacaaccagacctacaccttctg
cacccaccccaacctgatcgtgtgataa-648
CMV UL130 FL; (SEQ ID NO: 22)
MLRLLLRHHFHCLLLCAVWATPCLASPWSTLTANQNPSPPWSKLTYSKPHDAATFYCPFLYP
SPPRSPLQFSGFQRVSTGPECRNETLYLLYNREGQTLVERSSTWVKKVIWYLSGRNQTILQR
MPRTASKPSDGNVQISVEDAKIFGAHMVPKQTKLLRFVVNDGTRYQMCVMKLESWAHVFRDY
SVSFQVRLTFTEANNQTYTFCTHPNLIV--
CMV UL131 FL: (SEQ ID NO: 23)
1-
atgcggctgtgcagagtgtggctgtccgtgtgcctgtgtgccgtggtgctgggccagtgcca
gagagagacagccgagaagaacgactactaccgggtgccccactactgggatgcctgcagca
gagccctgcccgaccagacccggtacaaatacgtggagcagctcgtggacctgaccctgaac
taccactacgacgccagccacggcctggacaacttcgacgtgctgaagcggatcaacgtgac
cgaggtgtccctgctgatcagcgacttccggcggcagaacagaagaggcggcaccaacaagc
ggaccaccttcaacgccgctggctctctggcccctcacgccagatccctggaattcagcgtg
cggctgttcgccaactgataa-393
CMV UL131 FL; (SEQ ID NO: 24)
MRLCRVWLSVCLCAVVLGQCQRETAEKNDYYRVPHYWDACSRALPDQTRYKYVEQLVDLTLN
YHYDASHGLDNFDVLKRINVTEVSLLISDFRRQNRRGGTNKRTTFNAAGSLAPHARSLEFSV
RLFAN--
ParvoB19.Opti.VP1 (SEQ ID NO: 25)
acgcgtacaaaacaaaATGTCTAAGAAATCTGGTAAATGGTGGGAATCTGATGATAAATTTGCTAAGGC
TGTTTACCAACAATTTGTTGAATTTTACGAAAAGGTTACTGGTACTGATTTGGAATTGATTCAAATTTT
GAAGGATCATTACAACATTTCTTTGGATAATCCATTGGAAAATCCATCTTCATTGTTTGATTTGGTTGC
TAGAATTAAGAACAACTTGAAGAACTCTCCAGATTTGTATTCTCATCATTTCCAATCTCATGGTCAATT
GTCTGATCATCCACATGCTTTATCTTCATCTTCATCTCATGCTGAACCAAGAGGTGAAAATGCTGTTTT
ATCTTCTGAAGATTTGCATAAACCAGGTCAAGTTTCTGTTCAATTGCCAGGTACTAATTACGTTGGTCC
AGGTAATGAATTGCAAGCTGGTCCACCACAATCTGCTGTTGATTCTGCTGCTAGAATTCATGATTTCAG
ATACTCTCAATTGGCTAAGTTGGGTATTAATCCATATACTCATTGGACTGTTGCTGATGAAGAATTGTT
GAAGAACATTAAGAATGAAACTGGTTTTCAAGCTCAAGTTGTTAAAGATTACTTCACTTTGAAAGGTGC
TGCTGCTCCAGTTGCTCATTTTCAAGGTTCTTTGCCAGAAGTTCCAGCTTATAACGCTTCTGAAAAATA
TCCATCTATGACATCTGTTAATTCTGCTGAAGCATCTACTGGTGCAGGTGGAGGTGGTTCTAATTCTGT
TAAATCTATGTGGTCTGAAGGTGCTACTTTTTCTGCTAATTCAGTTACTTGTACTTTCTCTAGACAATT
CTTGATTCCATATGATCCAGAACATCATTACAAAGTTTTTTCACCAGCTGCTTCATCTTGTCATAATGC
TTCAGGTAAAGAAGCTAAGGTTTGTACTATTTCTCCAATTATGGGTTATTCTACTCCTTGGAGATACTT
GGATTTTAATGCTTTGAACTTGTTTTTTTCTCCATTGGAATTTCAACATTTGATTGAAAACTACGGTTC
TATTGCTCCAGATGCTTTGACTGTTACTATTTCTGAAATTGCTGTTAAGGATGTTACTGATAAAACAGG
TGGTGGTGTTCAAGTTACTGATTCTACTACTGGTAGATTGTGCATGTTGGTTGATCATGAATACAAATA
CCCATACGTTTTGGGTCAAGGTCAAGATACTTTGGCTCCAGAATTGCCAATTTGGGTTTATTTTCCACC
ACAATACGCTTATTTGACTGTTGGTGATGTTAATACTCAAGGTATTTCTGGTGATTCTAAAAAGTTGGC
TTCTGAAGAATCTGCTTTTTACGTTTTGGAACATTCTTCTTTTCAATTGTTGGGTACTGGTGGTACTGC
TTCTATGTCTTACAAATTTCCACCAGTTCCACCTGAAAATTTGGAAGGTTGTTCTCAACATTTTTACGA
AATGTACAATCCATTGTATGGTTCTAGATTGGGTGTTCCAGATACTTTGGGTGGTGATCCAAAATTTAG
ATCTTTGACTCATGAAGATCATGCTATTCAACCACAAAATTTCATGCCAGGTCCATTGGTTAATTCTGT
TTCTACTAAAGAAGGTGATTCTTCTAATACAGGTGCTGGTAAAGCATTGACTGGTTTGTCTACTGGTAC
TTCTCAAAACACTAGAATTTCTTTAAGACCAGGTCCAGTTTCACAACCATATCATCATTGGGATACTGA
TAAGTACGTTACTGGTATTAATGCTATTTCACATGGTCAAACTACTTATGGTAATGCTGAAGATAAAGA
ATATCAACAAGGTGTTGGTAGATTTCCAAACGAAAAAGAACAATTGAAACAATTGCAAGGTTTGAATAT
GCATACTTACTTTCCAAACAAAGGTACTCAACAATACACTGATCAAATTGAAAGACCATTGATGGTTGG
TTCTGTTTGGAAAGAAGAGCTTTGCATTATGAATCTCAATTGTGGTCTAAGATTCCAAATTTAGATGAT
TTCTTTCAAGACTCAATTTGCTGCTTTGGGTGGTTGGGGTTTGCATCAACCTCCACCACAAATTTTCTT
GAAGATTTTGCCACAATCTGGTCCAATTGGTGGTATTAAATCTATGGGTATTACTACTTTGGTTCAATA
TGCTGTTGGTATTATGACTGTTACAATGACTTTTAAGTTGGGTCCAAGAAAAGCTACAGGTAGATGGAA
TCCACAACCAGGTGTTTATCCACCACATGCTGCTGGTCATTTGCCTTACGTTTTGTATGATCCAACTGC
TACTGATGCTAAACAACATCATAGACATGGTTATGAAAAACCTGAAGAATTGTGGACTGCTAAATCTAG
AGTTCATCCATTGTAATGAgtcgac
ParvoB19.Opti.VP2 (SEQ ID NO: 26)
cctaggacaaaacaaaATGACATCTGTTAATTCTGCTGAAGCATCTACTGGTGCAGGTGGAGGTGGTTC
TAATTCTGTTAAATCTATGTGGTCTGAAGGTGCTACTTTTTCTGCTAATTCAGTTACTTGTACTTTCTC
TAGACAATTCTTGATTCCATATGATCCAGAACATCATTACAAAGTTTTTTCACCAGCTGCTTCATCTTG
TCATAATGCTTCAGGTAAAGAAGCTAAGGTTTGTACTATTTCTCCAATTATGGGTTATTCTACTCCTTG
GAGATACTTGGATTTTAATGCTTTGAACTTGTTTTTTTCTCCATTGGAATTTCAACATTTGATTGAAAA
CTACGGTTCTATTGCTCCAGATGCTTTGACTGTTACTATTTCTGAAATTGCTGTTAAGGATGTTACTGA
TAAAACAGGTGGTGGTGTTCAAGTTACTGATTCTACTACTGGTAGATTGTGCATGTTGGTTGATCATGA
ATACAAATACCCATACGTTTTGGGTCAAGGTCAAGATACTTTGGCTCCAGAATTGCCAATTTGGGTTTA
TTTTCCACCACAATACGCTTATTTGACTGTTGGTGATGTTAATACTCAAGGTATTTCTGGTGATTCTAA
AAAGTTGGCTTCTGAAGAATCTGCTTTTTACGTTTTGGAACATTCTTCTTTTCAATTGTTGGGTACTGG
TGGTACTGCTTCTATGTCTTACAAATTTCCACCAGTTCCACCTGAAAATTTGGAAGGTTGTTCTCAACA
TTTTTACGAAATGTACAATCCATTGTATGGTTCTAGATTGGGTGTTCCAGATACTTTGGGTGGTGATCC
AAAATTTAGATCTTTGACTCATGAAGATCATGCTATTCAACCACAAAATTTCATGCCAGGTCCATTGGT
TAATTCTGTTTCTACTAAAGAAGGTGATTCTTCTAATACAGGTGCTGGTAAAGCATTGACTGGTTTGTC
TACTGGTACTTCTCAAAACACTAGAATTTCTTTAAGACCAGGTCCAGTTTCACAACCATATCATCATTG
GGATACTGATAAGTACGTTACTGGTATTAATGCTATTTCACATGGTCAAACTACTTATGGTAATGCTGA
AGATAAAGAATATCAACAAGGTGTTGGTAGATTTCCAAACGAAAAAGAACAATTGAAACAATTGCAAGG
TTTGAATATGCATACTTACTTTCCAAACAAAGGTACTCAACAATACACTGATCAAATTGAAAGACCATT
GATGGTTGGTTCTGTTTGGAATAGAAGAGCTTTGCATTATGAATCTCAATTGTGGTCTAAGATTCCAAA
TTTAGATGATTCTTTCAAGACTCAATTTGCTGCTTTGGGTGGTTGGGGTTTGCATCAACCTCCACCACA
AATTTTCTTGAAGATTTTGCCACAATCTGGTCCAATTGGTGGTATTAAATCTATGGGTATTACTACTTT
GGTTCAATATGCTGTTGGTATTATGACTGTTACAATGACTTTTAAGTTGGGTCCAAGAAAAGCTACAGG
TAGATGGAATCCACAACCAGGTGTTTATCCACCACATGCTGCTGGTCATTTGCCTTACGTTTTGTATGA
TCCAACTGCTACTGATGCTAAACAACATCATAGACATGGTTATGAAAAACCTGAAGAATTGTGGACTGC
TAAATCTAGAGTTCATCCATTGTAATGAgcggccgc

1. An immunogenic composition comprising:
(i) a first polypeptide antigen that is not associated with a viral replicon particle (VRP), and
(ii) a self-replicating RNA molecule that encodes a second polypeptide antigen, wherein the self-replicating RNA cannot induce production of infectious viral particles and is provided with a delivery system;
wherein said first and second antigens are antigens from different pathogens.

2. The immunogenic composition of claim 1, wherein said first polypeptide antigen is a Cytomegalovirus (CMV) antigen.

3. The immunogenic composition of claim 1, wherein said second polypeptide antigen is a Parvovirus antigen.

4. The immunogenic composition of claim 1, wherein said second polypeptide antigen is in the form of a virus-like particle (VLP).

5. The immunogenic composition of claim 1, wherein said first polypeptide antigen is a soluble polypeptide, and said second polypeptide antigen is a soluble or membrane anchored polypeptide.

6. The immunogenic composition of claim 1, wherein the self-replicating RNA is an alphavirus-derived RNA replicon.

7. The immunogenic composition of claim 1, wherein the self-replicating RNA molecule comprises one or more modified nucleotides.

8. The immunogenic composition of claim 1, further comprising a cationic lipid, a liposome, a cochleate, a virosome, an immunestimulating complex, a microparticle, a microsphere,
a nanosphere, a unilamellar vesicle, a multilamellar vesicle, an oil-in-water emulsion, a water-in-oil emulsion, an emulsome, a polycationic peptide, or a cationic nanoemulsion.

9. The immunogenic composition of claim 1, wherein the RNA molecule is encapsulated in, bound to or adsorbed on a delivery system selected from a cationic lipid, a liposome, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, a polycationic peptide, a cationic nanoemulsion or combinations thereof.

10. The immunogenic composition of claim 1, wherein said first and second polypeptide antigens are independently derived from the group consisting of a viral pathogen, a bacterial pathogen, a fungal pathogen, a protozoan pathogen, and a multi-cellular parasitic pathogen.

11. The immunogenic composition of claim 1, wherein the first polypeptide antigen and second polypeptide antigen are both viral antigens.

12. The immunogenic composition of claim 11, wherein one viral antigen is an antigen from CMV.

13. The immunogenic composition of claim 11, wherein one viral antigen is a Parvovirus antigen.

14. The immunogenic composition of claim 13, wherein the Parvovirus antigen comprises an amino acid sequence encoded by NO: 25 or 26.

15. The immunogenic composition of claim 12, wherein the viral antigen is a gB antigen, a gH antigen, or a gL antigen.

16. The immunogenic composition of claim 12, wherein the viral antigen is a gH antigen or a gL antigen.

17. The immunogenic composition of claim 12, wherein the RNA molecule encodes a gH antigen and a gL antigen.

18. The immunogenic composition of claim 12, wherein the immunogenic composition comprises a gH polypeptide antigen and gL polypeptide antigen.

19. The immunogenic composition of claim 1, further comprising an adjuvant.

20. An immunogenic composition comprising: (i) a Parvovirus polypeptide antigen, and (ii) a self-replicating RNA molecule that encodes a CMV polypeptide antigen.

21. A method for inducing an immune response in a subject comprising administering to a subject in need thereof a therapeutically effective amount of a composition according to claim 1.

22. The composition of claim 1, wherein the self-replicating RNA is not encapsulated in a virus-like particle (VLP)
